(19) United States
(12) Patent Application Publication
(10) Pub. No.: US 2022/0257767 A1
(43) Pub. Date: Aug. 18, 2022

(54) IONIC LIQUIDS FOR DRUG DELIVERY

(71) Applicant: PRESIDENT AND FELLOWS OF HARVARD COLLEGE, Cambridge, MA (US)

(72) Inventors: Samir MITRAGOTRI, LEXINGTON, MA (US); Eden E.L. TANNER, CAMBRIDGE, MA (US)

(73) Assignee: PRESIDENT AND FELLOWS OF HARVARD COLLEGE, Cambridge, MA (US)

(21) Appl. No.: 17/600,305

(22) PCT Filed: Mar. 26, 2020
(86) PCT No.: PCT/US2020/024866

§ 371 Date: Sep. 30, 2021

Related U.S. Application Data
(60) Provisional application No. 62/828,539, filed on Apr. 3, 2019.

(51) Int. Cl. A61K 47/18; A61K 47/12; A61K 38/26; A61K 38/28; A61K 31/519; A61K 9/08; A61K 9/10; A61K 9/48; A61K 9/51

(52) U.S. Cl. CPC A61K 47/186; A61K 47/12; A61K 38/26; A61K 38/28; A61K 9/5107; A61K 9/08; A61K 9/10; A61K 9/4841; A61K 31/519

(57) ABSTRACT

The technology described herein is directed to ionic liquids and methods of drug delivery.

### CROSS-REFERENCE TO RELATED APPLICATIONS

This application claims benefit under 35 U.S.C. § 119(e) of U.S. Provisional Application No. 62/828,539 filed Apr. 3, 2019, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Mar. 24, 2020, is named 002806-094500WOPT_SL.txt and is 2,891 bytes in size.

### TECHNICAL FIELD

The technology described herein relates to ionic liquids for stabilization and delivery of active compounds.

### BACKGROUND

The uptake of many active compounds, e.g., pharmaceutically active compounds, can be improved by delivering the compounds in solvents. However, such approaches are often unsuitable for in vivo use because most such solvents demonstrate toxic side effects and/or act as irritants to the point of delivery. These toxic and irritant effects are severe enough to mitigate any increase in the uptake or performance of the active compound.

### SUMMARY

As demonstrated herein, the inventors have identified characteristics of ionic liquids that provide surprising superior active compound uptake kinetics. Accordingly, compositions and methods relating to these ionic liquids (ILs) with unexpectedly high efficacy are described herein.

In one aspect of any of the embodiments, described herein is a method of administering at least one active compound, the method comprising administering the active compound in combination with at least one ionic liquid (IL) comprising: i) a hydrophobic anion comprising a carboxylic acid having a pKa of at least 4.0 and a Log P of at least 1.0; and ii) a cation comprising a quaternary ammonium. In one aspect of any of the embodiments, described herein is a combination of at least one active compound and at least one ionic liquid comprising: i) a hydrophobic anion comprising a carboxylic acid having a pKa of at least 4.0 and a Log P of at least 1.0; and ii) a cation comprising a quaternary ammonium for use in a method of drug delivery.

In one aspect of any of the embodiments, described herein is a method of reducing weight/weight gain or treating obesity, diabetes, ulcers, cancer, or fibrosis in a subject in need thereof, the method comprising administering a composition comprising at least one ionic liquid comprising: i) a hydrophobic anion comprising a carboxylic acid having a pKa of at least 4.0 and a Log P of at least 1.0; and ii) a cation comprising a quaternary ammonium to the subject. In one aspect of any of the embodiments, described herein is a composition comprising at least one ionic liquid comprising: i) a hydrophobic anion comprising a carboxylic acid having a pKa of at least 4.0 and a Log P of at least 1.0; and ii) a cation comprising a quaternary ammonium for use in a method of reducing weight/weight gain or treating obesity, diabetes, ulcers, cancer, or fibrosis in a subject in need thereof. In some embodiments, the subject is not administered, and/or the composition does not comprise a therapeutic agent or active compound other than the at least one ionic liquid. In some embodiments, the subject is administered (e.g., in the same formulation as the at least one IL or in a separate formulation), and/or the composition further comprises a therapeutic agent or active compound other than the at least one ionic liquid.

In one aspect, provided herein is a composition comprising at least one ionic liquid comprising: i) a hydrophobic anion comprising a carboxylic acid having a pKa of at least 4.0 and a Log P of at least 1.0; and ii) a cation comprising a quaternary ammonium. In some embodiments, the composition further comprises a therapeutic agent or active compound in combination with the at least one ionic liquid.

In some embodiments of any of the aspects, the anion has a pKa of at least 4.5. In some embodiments of any of the aspects, the anion has a pKa of at least 5.0. In some embodiments of any of the aspects, the anion has a Log P of at least 2.0. In some embodiments of any of the aspects, the anion has a Log P of at least 2.5. In some embodiments of any of the aspects, the anion has a Log P of at least 2.75.

In some embodiments of any of the aspects, the anion comprises a carbon chain backbone of at least 8 carbons. In some embodiments of any of the aspects, the anion is an alkene. In some embodiments of any of the aspects, the anion is geranic acid, octanoic acid, or citronellic acid.

In some embodiments of any of the aspects, the cation has a molar mass equal to or greater than choline. In some embodiments of any of the aspects, the cation has a molar mass greater than choline. In some embodiments of any of the aspects, the quarternary ammonium has the structure of NR₄⁺ and at least one R group comprises a hydroxy group. In some embodiments of any of the aspects, the quarternary ammonium has the structure of NR₄⁺ and only one R group comprises a hydroxy group. In some embodiments of any of the aspects, the cation is C1, C6, or C7.

In some embodiments of any of the aspects, the cation is selected from choline, C1, C6, and C7 and the anion is citronellic acid. In some embodiments of any of the aspects, the cation is C1 and the anion is citronellic acid. In some embodiments of any of the aspects, the cation is selected from C1, C6, and C7 and the anion is geranic acid. In some embodiments of any of the aspects, the ionic liquid is choline: citronellic acid, C1: geranic acid, or C1: citronellic acid. In some embodiments of any of the aspects, the ionic liquid is not CAGE.

In some embodiments of any of the aspects, the ionic liquid has less than 20, less than 10, or less than 5 cross peaks as measured by Nuclear Overhauser Effect SpectroscopY (NOESY).

In some embodiments of any of the aspects, the administration is transdermal. In some embodiments of any of the aspects, the administration is transdermal, to a mucus membrane, oral, subcutaneous, intradermal, parenteral, intratumoral, or intravenous. In some embodiments of any of the aspects, the composition or combination is formulated for transdermal administration. In some embodiments of any of the aspects, the composition or combination is formulated for transdermal, to a mucus membrane, oral, subcutaneous, intradermal, parenteral, intratumoral, or intravenous administration. In some embodiments of any of the aspects, the mucus membrane is nasal, oral, or vaginal.

In some embodiments of any of the aspects, the ionic liquid is at a concentration of at least 0.1% w/v. In some embodiments of any of the aspects, the ionic liquid is at a concentration of from about 10 to about 70% w/v. In some embodiments of any of the aspects, the ionic liquid is at a concentration of from about 30 to about 50% w/v. In some embodiments of any of the aspects, the ionic liquid is at a concentration of from about 30 to about 40% w/v. In some embodiments of any of the aspects, the ionic liquid comprises a ratio of cation to anion of from about 2:1 to about 1:10. In some embodiments of any of the aspects, the ionic liquid comprises a ratio of cation to anion of from about 1:1 to about 1:4. In some embodiments of any of the aspects, the ionic liquid comprises a ratio of cation to anion of about 1:2. In some embodiments of any of the aspects, the ionic liquid has a cation:anion ratio of less than 1:1. In some embodiments of any of the aspects, the ionic liquid has a cation:anion ratio comprising an excess of anion.

In some embodiments of any of the aspects, the active compound is hydrophobic. In some embodiments of any of the aspects, the active compound is hydrophilic. In some embodiments of any of the aspects, the active compound comprises a polypeptide. In some embodiments of any of the aspects, the active compound has a molecular weight of greater than 450. In some embodiments of any of the aspects, the active compound has a molecular weight of greater than 500. In some embodiments of any of the aspects, the active compound comprises an antibody or antibody reagent. In some embodiments of any of the aspects, the active compound comprises insulin, acarbose, ruxolitinib, or a GLP-1 polypeptide or mimetic or analog thereof.

In some embodiments of any of the aspects, the combination and/or composition is administered once. In some embodiments of any of the aspects, the combination and/or composition is administered in multiple doses. In some embodiments of any of the aspects, the active compound and/or composition is provided at a dosage of 1-20 mg/kg.

In some embodiments of any of the aspects, the active compound and the ionic liquid are further in combination with at least one non-ionic surfactant. In some embodiments of any of the aspects, the combination and/or composition further comprises a further pharmaceutically acceptable carrier. In some embodiments of any of the aspects, the administration is oral and the combination and/or composition is provided in a degradable capsule. In some embodiments of any of the aspects, the combination is an admixture. In some embodiments of any of the aspects, the combination and/or composition is provided in one or more nanoparticles. In some embodiments of any of the aspects, the combination is provided in the form of one or more nanoparticles comprising the active compound, the nanoparticles in solution or suspension in a composition comprising the ionic liquid.

### BRIEF DESCRIPTION OF THE DRAWINGS

FIG. 1A depicts the chemical structures of Choline and Geranic acid. FIG. 1B depicts CAGE prepared by salt metathesis of choline bicarbonate and geranic acid at a molar ratio of 1:2.

FIG. 2 depicts the two drugs assessed in this study: acarbose and ruxolitinib. Acarbose was used as model hydrophilic drug (MW: 646, Log P: −6.8) and ruxolitinib was used as a model hydrophobic drug (MW: 306 and Log P: 2.9).

FIG. 3 depicts the amount of acarbose or ruxolitinib delivered into and across the skin after topical application from various CAGE compositions. Both drugs were dissolved in CAGE at a concentration of 1 mg/ml (N=3, error bars are SEM).

FIG. 4 depicts the anions used as alternatives to geranic acid. The anions were selected to cover a range of molecular weights, pKa and Log P values.

FIGS. 5A-5D depict the relationships between the overall transport rank of the anionic alternatives and the (FIG. 5A) molecular weight, (FIG. 5B) pKa, (FIG. 5C) Log P and (FIG. 5D) the number of carbons in the anion.

FIGS. 6A-6B depict the NOESY spectra of (FIG. 6A) choline: citronellic acid and (FIG. 6B) choline: glutaric acid. The circled cross peaks represent parts of the molecule are within 5 nm of each other. The number of cross peaks exhibit a significant difference among the ILs studied.

FIG. 7 depicts the correlation between the transport ranking and number of NOSEY cross correlation peaks. NOSEY data for choline: citronellic acid and choline: glutaric acid is taken from FIGS. 6A-6B. NOSEY data for other ILs is shown in SI. Transport rankings and abbreviations are in Table 2.

FIG. 8 depicts the range of quaternary ammoniums synthesized as cationic alternatives to choline.

FIG. 9A depicts the transport of ruxolitinib (1 mg/ml) to different portions of the skin over a 24-hour period by ionic liquid (L-R where horizontal stripes are C1: citronellic acid, diagonal stripes are C1: geranaic acid, solid black is CAGE, dotted is choline: citronellic acid). E is epidermis, D is dermis, A is acceptor fluid. Error bars are SEM (n=3). FIG. 9B depicts 2D NOESY spectrum of C1: citronellic acid showing one cross-peak (circled).

FIG. 10 depicts the transport of ruxolitinib (1 mg/ml) to different portions of the skin over a 24-hour period by PBS (white column, none detected) and CAGE ionic liquid (2:1 is shown as diagonal stripes, 1:1 is solid black, 1:2 is horizontal stripes, and 1:4 is dotted). E is epidermis, D is dermis, A is acceptor fluid. Error bars are SE of the mean. Note that pure methanol was used to extract ruxolitinib from the skin, which also extracted significant SC lipids simultaneously, dwarfing the drug peak seen by HPLC. The SC data was thus excluded from the ruxolitinib analysis.

FIG. 11 depicts transport of acarbose (1 mg/ml) to different portions of the skin over a 24-hour period by PBS (white column, none detected) and CAGE ionic liquid (2:1 is shown as diagonal stripes, 1:1 is solid black, 1:2 is horizontal stripes, and 1:4 is dotted). SC is stratum corneum, E is epidermis, D is dermis, A is acceptor fluid. Error bars are SE of the mean.

FIG. 12 depicts transport of acarbose to different portions of the skin over a 24-hour period by ionic liquid (L-R where solid black is CAGE, dotted is choline citronellate, diagonal downward is choline hexanoate, wave is choline decanoate, horizontal line is choline salicylate, weave is choline glutarate, confetti is choline glycolate, diagonal upward is choline octenoate, and grid is choline octanoate). SC is stratum corneum, E is epidermis, D is dermis, A is acceptor fluid. Error bars are SE of the mean.

FIG. 13 depicts transport of ruxolitinib (1 mg/ml) to different portions of the skin over a 24-hour period by ionic liquid (L-R where solid black is CAGE, horizontal line is choline salicylate, weave is choline glutarate, confetti is choline glycolate, dotted is choline citronellate, diagonal downward is choline hexanoate, wave is choline decanoate, diagonal upward is choline octenoate, and grid is choline octanoate). E is epidermis, D is dermis, A is acceptor fluid. Error bars are SE of the mean.

FIG. 14 depicts 2D NOESY spectrum of Choline Octenoate (1:2).

FIG. 15 depicts 2D NOESY spectrum of Choline Octanoate (1:2).

FIG. 16 depicts 2D NOESY spectrum of Choline Decanoate (1:2).

FIG. 17 depicts 2D NOESY spectrum of Choline Salicylate (1:2).

FIG. 18 depicts 2D NOESY spectrum of Choline Glycolate (1:2).

FIG. 19 depicts 2D NOESY spectrum of Choline hexanoate (1:2).

FIG. 20 depicts 2D NOESY spectrum of Choline geranate (CAGE, 1:2) (from Tanner et al., 2018).

FIG. 21 depicts 2D NOESY spectrum of Choline alternative 1 with geranic acid (1:2).

FIG. 22 depicts intestinal administration of insulin with the indicated ionic liquids. Inulin was mixed with ionic liquids and administered in the intestine. Blood glucose concentrations and plasma insulin concentrations were measured. As used in the legend of FIG. 22, “C—” refers to choline as the cation in an ionic liquid.

### DETAILED DESCRIPTION

The data provided herein demonstrate that the anion of an ionic liquid (IL) exerts the predominant influence on whether an active agent will be transported across a biological barrier (e.g., an epithelial layer, such as the dermis). Anions with higher hydrophobicity and higher mass will provide improved drug delivery characteristics than anions with lower hydrophobicity and lower mass. In selecting a cation to pair with the anion, the primary concern is that the cation not associate too closely with the anion—close association causes the anion to be retained on the initial side of the biological barrier.

Accordingly, in one aspect of any of the embodiments, described herein is an ionic liquid comprising: i) a hydrophobic anion comprising a carboxylic acid having a pKa of at least 4.0 and/or a Log P of at least 1.0; and ii) a cation comprising a quaternary ammonium. In one aspect of any of the embodiments, described herein is an active compound in combination with at least one ionic liquid, the ionic liquid comprising: i) a hydrophobic anion comprising a carboxylic acid having a pKa of at least 4.0 and/or a Log P of at least 1.0; and ii) a cation comprising a quaternary ammonium. Accordingly, in one aspect of any of the embodiments, described herein is a method of administering at least one active compound, the method comprising administering the active compound in combination with at least one ionic liquid, the ionic liquid comprising: i) a hydrophobic anion comprising a carboxylic acid having a pKa of at least 4.0 and/or a Log P of at least 1.0; and ii) a cation comprising a quaternary ammonium.

The term “ionic liquids (ILs)” as used herein refers to organic salts or mixtures of organic salts which are in liquid state at room temperature. This class of solvents has been shown to be useful in a variety of fields, including in industrial processing, catalysis, pharmaceuticals, and electrochemistry. The ionic liquids contain at least one anionic and at least one cationic component. Ionic liquids can comprise an additional hydrogen bond donor (i.e. any molecule that can provide an —OH or an —NH group), examples include but are not limited to alcohols, fatty acids, and amines. The at least one anionic and at least one cationic component may be present in any molar ratio. Exemplary molar ratios (cation:anion) include but are not limited to 1:1, 1:2, 2:1, 1:3, 3:1, 2:3, 3:2, and ranges between these ratios. For further discussion of ionic liquids, see, e.g., Hough, et ah, “The third evolution of ionic liquids: active pharmaceutical ingredients”, New Journal of Chemistry, 31: 1429 (2007) and Xu, et al., “Ionic Liquids: Ion Mobilities, Glass Temperatures, and Fragilities”, Journal of Physical Chemistry B, 107(25): 6170-6178 (2003); each of which is incorporated by reference herein in its entirety. In some embodiments of any of the aspects, the ionic liquid or solvent exists as a liquid below 100° C. In some embodiments of any of the aspects, the ionic liquid or solvent exists as a liquid at room temperature.

As demonstrated herein, anions with higher hydrophobicity and higher mass will provide improved drug delivery characteristics than anions with lower hydrophobicity and lower mass. In some embodiments of any of the aspects, the anion of an IL described herein is hydrophobic. In some embodiments of any of the aspects, the anion of an IL described herein is comprises a carboxylic acid.

In some embodiments of any of the aspects, the anion of an IL described herein is has a pKa of at least 4.0, e.g., 4.0 or greater. In some embodiments of any of the aspects, the anion of an IL described herein is has a pKa of at least 4.5, e.g., 4.5 or greater. In some embodiments of any of the aspects, the anion of an IL described herein is has a pKa of at least 5.0, e.g., 5.0 or greater. In some embodiments of any of the aspects, the anion of an IL described herein is has a pKa of at about least 4.0, e.g., about 4.0 or greater. In some embodiments of any of the aspects, the anion of an IL described herein is has a pKa of at least about 4.5, e.g., about 4.5 or greater. In some embodiments of any of the aspects, the anion of an IL described herein is has a pKa of at least about 5.0, e.g., about 5.0 or greater.

In some embodiments of any of the aspects, the anion has a pKa of at least 4.895. In some embodiments of any of the aspects, the anion has a pKa of 4.5-5.5. In some embodiments of any of the aspects, the anion has a pKa of 4.895-5.19.

In some embodiments of any of the aspects, the anion has a pKa of at least about 4.895. In some embodiments of any of the aspects, the anion has a pKa of about 4.5 to about 5.5. In some embodiments of any of the aspects, the anion has a pKa of about 4.895 to about 5.19.

Hydrophobicity may be assessed by analysis of log P. “Log P” refers to the logarithm of P (Partition Coefficient). P is a measure of how well a substance partitions between a lipid (oil) and water. P itself is a constant. It is defined as the ratio of concentration of compound in aqueous phase to the concentration of compound in an immiscible solvent, as the neutral molecule.

Partition Coefficient, P=[Organic]/[Aqueous] where [ ]=concentration

Log P=log₁₀ (Partition Coefficient)=log₁₀ P

In practice, the Log P value will vary according to the conditions under which it is measured and the choice of partitioning solvent. A Log P value of 1 means that the concentration of the compound is ten times greater in the organic phase than in the aqueous phase. The increase in a log P value of 1 indicates a ten fold increase in the concentration of the compound in the organic phase as compared to the aqueous phase.

In some embodiments of any of the aspects, the anion of an IL described herein is has a Log P of at least 1.0, e.g., 1.0 or greater. In some embodiments of any of the aspects, the anion of an IL described herein is has a Log P of at least 2.0, e.g., 2.0 or greater. In some embodiments of any of the aspects, the anion of an IL described herein is has a Log P of at least 2.5 e.g., 2.5 or greater. In some embodiments of any of the aspects, the anion of an IL described herein is has a Log P of at least 2.75, e.g., 2.75 or greater. In some embodiments of any of the aspects, the anion of an IL described herein is has a Log P of at least about 1.0, e.g., about 1.0 or greater. In some embodiments of any of the aspects, the anion of an IL described herein is has a Log P of at least about 2.0, e.g., about 2.0 or greater. In some embodiments of any of the aspects, the anion of an IL described herein is has a Log P of at least about 2.5 e.g., about 2.5 or greater. In some embodiments of any of the aspects, the anion of an IL described herein is has a Log P of at least about 2.75, e.g., about 2.75 or greater.

In some embodiments of any of the aspects, the anion of an IL described herein is has a pKa of at least 4.0 and a Log P of at least 1.0. In some embodiments of any of the aspects, the anion of an IL described herein is has a pKa of at least 4.0 and a Log P of at least 2.0. In some embodiments of any of the aspects, the anion of an IL described herein is has a pKa of at least 4.0 and a Log P of at least 2.5. In some embodiments of any of the aspects, the anion of an IL described herein is has a pKa of at least 4.0 and a Log P of at least 2.75.

In some embodiments of any of the aspects, the anion of an IL described herein is has a pKa of at least 4.5 and a Log P of at least 1.0. In some embodiments of any of the aspects, the anion of an IL described herein is has a pKa of at least 4.5 and a Log P of at least 2.0. In some embodiments of any of the aspects, the anion of an IL described herein is has a pKa of at least 4.5 and a Log P of at least 2.5. In some embodiments of any of the aspects, the anion of an IL described herein is has a pKa of at least 4.5 and a Log P of at least 2.75.

In some embodiments of any of the aspects, the anion of an IL described herein is has a pKa of at least 5.0 and a Log P of at least 1.0. In some embodiments of any of the aspects, the anion of an IL described herein is has a pKa of at least 5.0 and a Log P of at least 2.0. In some embodiments of any of the aspects, the anion of an IL described herein is has a pKa of at least 5.0 and a Log P of at least 2.5. In some embodiments of any of the aspects, the anion of an IL described herein is has a pKa of at least 5.0 and a Log P of at least 2.75.

In some embodiments of any of the aspects, the anion has a Log P of at least 2.75. In some embodiments of any of the aspects, the anion has a Log P of at least 2.8. In some embodiments of any of the aspects, the anion has a Log P of 2.5-3.5. In some embodiments of any of the aspects, the anion has a Log P of 2.8-3.01.

In some embodiments of any of the aspects, the anion has a Log P of at least about 2.75. In some embodiments of any of the aspects, the anion has a Log P of at least about 2.8. In some embodiments of any of the aspects, the anion has a Log P of about 2.5 to about 3.5. In some embodiments of any of the aspects, the anion has a Log P of about 2.8 to about 3.01.

In some embodiments of any of the aspects, the carboxylic acid comprises a carbon backbone chain having 8 carbons and has a Log P greater than are equal to 2.8 and a pKa between 4.8 and 5.2. In some embodiments of any of the aspects, the carboxylic acid has a Log P greater than or equal to 2.9 and a pKa between 4.8 and 5.1.

The pKa and Log P values for anions are known in the art and/or can be calculated by one of skill in the art. For example, PubChem and SpiderChem provide these values for various anions and chemical manufacturers typically provide them as part of the catalog listings for their products. pKa and Log P values for exemplary anions are provided in Table 5 herein.

In some embodiments of any of the aspects, the carboxylic acid comprises a carbon chain of at least 6 carbons. In some embodiments of any of the aspects, the carboxylic acid comprises a carbon chain of at least 7 carbons. In some embodiments of any of the aspects, the carboxylic acid comprises a carbon chain of at least 8 carbons. In some embodiments of any of the aspects, the carboxylic acid comprises a carbon chain of at least 9 carbons. In some embodiments of any of the aspects, the carboxylic acid comprises a carbon chain of at least 10 carbons. In some embodiments of any of the aspects, the carboxylic acid comprises a carbon chain of at least 11 carbons.

In some embodiments of any of the aspects, the anion is an alkane. In some embodiments of any of the aspects, the anion is an alkene. In some embodiments of any of the aspects, the anion comprises a single carboxyl group. In some embodiments of any of the aspects, the carbon chain of the carboxylic acid comprises one or more substituent groups. In some embodiments of any of the aspects, the carbon chain backbone of the carboxylic acid comprises one or more substituent groups, wherein each substituent group comprises at least one carbon atom. In some embodiments of any of the aspects, the carbon chain backbone of the carboxylic acid comprises one or more substituent groups, wherein at least one substituent group comprises a methyl group. In some embodiments of any of the aspects, the carbon chain backbone of the carboxylic acid comprises two substituent groups, wherein each substituent group comprises at least one carbon atom. In some embodiments of any of the aspects, the carbon chain backbone of the carboxylic acid comprises two substituent groups, wherein one substituent group comprises a methyl group. In some embodiments of any of the aspects, the carbon chain backbone of the carboxylic acid comprises two substituent groups, wherein each substituent group comprises a methyl group.

In some embodiments of any of the aspects, the anion is an unsubstituted alkane. In some embodiments of any of the aspects, the anion is an unsubstituted alkene. In some embodiments of any of the aspects, the carbon chain backbone of the carboxylic acid comprises one or more substituent groups. In some embodiments of any of the aspects, the carbon chain of the carboxylic acid comprises one or more substituent groups, wherein each substituent group comprises at least one carbon atom. In some embodiments of any of the aspects, the carbon chain of the carboxylic acid comprises one or more substituent groups, wherein each substituent group is alkyl, aryl, heteroalkayl, heteroaryl, alkane, or alkene. In some embodiments of any of the aspects, the carbon chain of the carboxylic acid comprises one or more substituent groups, wherein each substituent group is unsubstituted alkyl, unsubstituted aryl, unsubstituted heteroalkayl, unsubstituted heteroaryl, unsubstituted alkane, or unsubstituted alkene.

In some embodiments of any of the aspects, the carboxylic acid comprises a carbon backbone chain having 8 carbons, is optionally a mono-alkene, and optionally has two substituents. In some embodiments of any of the aspects, at least one of the substituents is a methyl group. In some embodiments of any of the aspects, both of the substituents is a methyl group. In some embodiments of any of the aspects, the carboxylic acid is selected from the group consisting of: octanoic acid; 2-octenoic acid; 3-octenoic acid; 4-octenoic acid; 5-octenoic acid; 6-octenoic acid; 7-octenoic acid; 2,2-dimethyloctanoic acid; 2,3-dimethyloctanoic acid; 2,4-dimethyloctanoic acid; 2,5-dimethyloctanoic acid; 2,6-dimethyloctanoic acid; 2,7-dimethyloctanoic acid; 3,3-dimethyloctanoic acid; 3,4-dimethyloctanoic acid; 3,5-dimethyloctanoic acid; 3,6-dimethyloctanoic acid; 3,7-dimethyloctanoic acid; 4,4-dimethyloctanoic acid; 4,5-dimethyloctanoic acid; 4,6-dimethyloctanoic acid; 4,7-dimethyloctanoic acid; 5,5-dimethyloctanoic acid; 5,6-dimethyloctanoic acid; 5,7-dimethyloctanoic acid; 6,6-dimethyloctanoic acid; 6,7-dimethyloctanoic acid; 7,7-dimethyloctanoic acid; 2,3-dimethyl-2-octenoic acid; 2,4-dimethyl-2-octenoic acid; 2,5-dimethyl-2-octenoic acid; 2,6-dimethyl-2-octenoic acid; 2,7-dimethyl-2-octenoic acid; 3,4-dimethyl-2-octenoic acid; 3,5-dimethyl-2-octenoic acid; 3,6-dimethyl-2-octenoic acid; 3,7-dimethyl-2-octenoic acid; 4,4-dimethyl-2-octenoic acid; 4,5-dimethyl-2-octenoic acid; 4,6-dimethyl-2-octenoic acid; 4,7-dimethyl-2-octenoic acid; 5,5-dimethyl-2-octenoic acid; 5,6-dimethyl-2-octenoic acid; 5,7-dimethyl-2-octenoic acid; 6,6-dimethyl-2-octenoic acid; 6,7-dimethyl-2-octenoic acid, 7,7-dimethyl-2-octenoic acid; 2,2-dimethyl-3-octenoic acid; 2,3-dimethyl-3-octenoic acid; 2,4-dimethyl-3-octenoic acid; 2,5-dimethyl-3-octenoic acid; 2,6-dimethyl-3-octenoic acid; 2,7-dimethyl-3-octenoic acid; 3,4-dimethyl-3-octenoic acid; 3,5-dimethyl-3-octenoic acid; 3,6-dimethyl-3-octenoic acid; 3,7-dimethyl-3-octenoic acid; 4,5-dimethyl-3-octenoic acid; 4,6-dimethyl-3-octenoic acid; 4,7-dimethyl-3-octenoic acid; 5,5-dimethyl-3-octenoic acid; 5,6-dimethyl-3-octenoic acid; 5,7-dimethyl-3-octenoic acid; 6,6-dimethyl-3-octenoic acid; 6,7-dimethyl-3-octenoic acid; 7,7-dimethyl-3-octenoic acid; 2,2-dimethyl-4-octenoic acid; 2,3-dimethyl-4-octenoic acid; 2,4-dimethyl-4-octenoic acid; 2,5-dimethyl-4-octenoic acid; 2,6-dimethyl-4-octenoic acid; 2,7-dimethyl-4-octenoic acid; 3,3-dimethyl-4-octenoic acid; 3,4-dimethyl-4-octenoic acid; 3,5-dimethyl-4-octenoic acid; 3,6-dimethyl-4-octenoic acid; 3,7-dimethyl-4-octenoic acid; 4,5-dimethyl-4-octenoic acid; 4,6-dimethyl-4-octenoic acid; 4,7-dimethyl-4-octenoic acid; 5,6-dimethyl-4-octenoic acid; 5,7-dimethyl-4-octenoic acid; 6,6-dimethyl-4-octenoic acid; 6,7-dimethyl-4-octenoic acid; 7,7-dimethyl-4-octenoic acid; 2,2-dimethyl-5-octenoic acid; 2,3-dimethyl-5-octenoic acid; 2,4-dimethyl-5-octenoic acid; 2,5-dimethyl-5-octenoic acid; 2,6-dimethyl-5-octenoic acid; 2,7-dimethyl-5-octenoic acid; 3,3-dimethyl-5-octenoic acid; 3,4-dimethyl-5-octenoic acid; 3,5-dimethyl-5-octenoic acid; 3,6-dimethyl-5-octenoic acid; 3,7-dimethyl-5-octenoic acid; 4,4-dimethyl-5-octenoic acid; 4,5-dimethyl-5-octenoic acid; 4,6-dimethyl-5-octenoic acid; 4,7-dimethyl-5-octenoic acid; 5,6-dimethyl-5-octenoic acid; 5,7-dimethyl-5-octenoic acid; 6,7-dimethyl-5-octenoic acid; 7,7-dimethyl-5-octenoic acid; 2,2-dimethyl-6-octenoic acid; 2,3-dimethyl-6-octenoic acid; 2,4-dimethyl-6-octenoic acid; 2,5-dimethyl-6-octenoic acid; 2,6-dimethyl-6-octenoic acid; 2,7-dimethyl-6-octenoic acid; 3,3-dimethyl-6-octenoic acid; 3,4-dimethyl-6-octenoic acid; 3,5-dimethyl-6-octenoic acid; 3,6-dimethyl-6-octenoic acid; 3,7-dimethyl-6-octenoic acid (citranellic acid); 4,4-dimethyl-6-octenoic acid; 4,5-dimethyl-6-octenoic acid; 4,6-dimethyl-6-octenoic acid; 4,7-dimethyl-6-octenoic acid; 5,5-dimethyl-6-octenoic acid; 5,6-dimethyl-6-octenoic acid; 5,7-dimethyl-6-octenoic acid; 6,7-dimethyl-6-octenoic acid; 2,2-dimethyl-7-octenoic acid; 2,3-dimethyl-7-octenoic acid; 2,4-dimethyl-7-octenoic acid; 2,5-dimethyl-7-octenoic acid; 2,6-dimethyl-7-octenoic acid; 2,7-dimethyl-7-octenoic acid; 4,4-dimethyl-7-octenoic acid; 3,4-dimethyl-7-octenoic acid; 3,5-dimethyl-7-octenoic acid; 3,6-dimethyl-7-octenoic acid; 3,7-dimethyl-7-octenoic acid; 4,4-dimethyl-7-octenoic acid; 4,5-dimethyl-7-octenoic acid; 4,6-dimethyl-7-octenoic acid; 4,7-dimethyl-7-octenoic acid; 5,5-dimethyl-7-octenoic acid; 5,6-dimethyl-7-octenoic acid; 5,7-dimethyl-7-octenoic acid; 6,6-dimethyl-7-octenoic acid; 6,7-dimethyl-7-octenoic acid; and isomers thereof. In some embodiments of any of the aspects, the carboxylic acid is selected from the group consisting of: octanoic acid; 2-octenoic acid; 3-octenoic acid; 4-octenoic acid; 5-octenoic acid; 6-octenoic acid; 7-octenoic acid; 2,2-dimethyloctanoic acid; 2,4-dimethyloctanoic acid; 2,5-dimethyloctanoic acid; 2,6-dimethyloctanoic acid; 2,7-dimethyloctanoic acid; 3,3-dimethyloctanoic acid; 3,5-dimethyloctanoic acid; 3,6-dimethyloctanoic acid; 3,7-dimethyloctanoic acid; 4,4-dimethyloctanoic acid; 4,5-dimethyloctanoic acid; 4,6-dimethyloctanoic acid; 5,5-dimethyloctanoic acid; 5,6-dimethyloctanoic acid; 5,7-dimethyloctanoic acid; 6,6-dimethyloctanoic acid; 7,7-dimethyloctanoic acid; 3,7-dimethyl-2-octenoic acid; 3,7-dimethyl-3-octenoic acid; 3,7-dimethyl-4-octenoic acid; 2,7-dimethyl-6-octenoic acid; 3,7-dimethyl-6-octenoic acid (citranellic acid); 2,2-dimethyl-7-octenoic acid; 2,3-dimethyl-7-octenoic acid; and isomers thereof. In some embodiments of any of the aspects, the carboxylic acid is selected from the group consisting of citranellic acid, octanoic acid, 2-octenoic acid and isomers thereof. In some embodiments of any of the aspects, the carboxylic acid is selected from the group consisting of citranellic acid, octanoic acid or trans-2-octenoic acid. In some embodiments of any of the aspects, octenoic acid as used herein (for example in Table 5) refers to trans-2-octenoic acid.

In some embodiments of any of the aspects, the carboxylic acid comprises a carbon backbone chain having 8 carbons and is optionally a mono-alkene. In some embodiments of any of the aspects, the carbon backbone chain of the carboxylic acid is not substituted. In some embodiments of any of the aspects, the carboxylic acid is selected from the group consisting of octanoic acid, 2-octenoic acid, 3-octenoic acid, 4-octenoic acid, 5-octenoic acid, 6-octenoic acid, 7-octenoic acid and isomers thereof. In some options, the carboxylic acid is octanoic acid or trans-2-octenoic acid (octenoic acid).

Exemplary, non-limiting anions are provided in Table 5 below.

TABLE 5

| | LogP | pKa |
|---|---|---|
| Group 1 | | |
| Geranic Acid | 2.72 | 5.26 |
| Citronellic Acid | 2.8 | 5.19 |
| Octenoic Acid | 2.9 | 5.05 |
| Decenoic Acid | 4.02 | 5.03 |
| (9Z)-octadec-9-enoic acid | 6.5 | 5.02 |
| Group 2 | | |
| Octanoic Acid | 3.01 | 4.895 |
| Decanoic Acid | 4.09 | 4.9 |
| (9Z, 12Z)-octadeca-9,12- | 7.05 | 4.77 |
| dienoic acid | | |
| (R)-5-(1,2-dithiolan-3- | 2.1 | 5.10 |
| yl)pentanoic acid | | |
| Group 3 | | |
| Hexenoic Acid | 1.8 | 5.13 |
| Group 4 | | |
| Hexanoic Acid | 1.92 | 4.88 |
| 3-methylbutanoic acid | 1.2 | 4.77 |
| Nonanedioic Acid | 1.57 | 4.55 |
| Pentanoic acid | 1.39 | 4.84 |
| Group 5 | | |
| 2-hydroxyoctanoic acid | 1.8 | 4.42 |
| (E)-3-(4-hydroxy-3-methoxy- | 1.51 | 4.42 |
| phenyl)prop-2-enoic acid | | |
| Group 6 | | |
| 2-ethylhexyl sulfate | 3.10 | |
| 2-(dimethylamino)ethanol | −0.55 | 9.3 |
| Group 7 | | |
| 8-hydroxycapric acid | 2.2 | |
| 2-methylpropanoic acid | 0.73 | 4.84 |
| Ascorbic Acid | −1.85 | 4.7 |
| Butanoic acid | 0.79 | 4.82 |
| Salicylic Acid | 2.2 | 2.97 |
| Group 8 | | |
| Hydroxyl(phenyl)acetic acid | 1.2 | 3.41 |
| Glutaric Acid | −0.29 | 4.34 |
| Adipic acid | 0.08 | 4.4 |
| Group 9 | | |
| Octanoic Acid | 3.01 | 4.895 |
| Citronellic Acid | 2.8 | 5.19 |
| Octenoic Acid | 2.9 | 5.05 |
| Group 10 | | |
| Octanoic Acid | 3.01 | 4.895 |
| Octenoic Acid | 2.9 | 5.05 |

In some embodiments of any of the aspects, the anion is selected from Table 5. In some embodiments of any of the aspects, the anion is selected from Group 1 of Table 5. In some embodiments of any of the aspects, the anion is selected from Group 2 of Table 5. In some embodiments of any of the aspects, the anion is selected from Group 3 of Table 5. In some embodiments of any of the aspects, the anion is selected from Group 4 of Table 5. In some embodiments of any of the aspects, the anion is selected from Group 5 of Table 5. In some embodiments of any of the aspects, the anion is selected from Group 6 of Table 5. In some embodiments of any of the aspects, the anion is selected from Group 7 of Table 5. In some embodiments of any of the aspects, the anion is selected from Group 8 of Table 5. In some embodiments of any of the aspects, the anion is selected from Groups 1-2 of Table 5. In some embodiments of any of the aspects, the anion is selected from Groups 1-3 of Table 5. In some embodiments of any of the aspects, the anion is selected from Groups 1-4 of Table 5. In some embodiments of any of the aspects, the anion is selected from Groups 1-5 of Table 5. In some embodiments of any of the aspects, the anion is selected from Groups 1-6 of Table 5. In some embodiments of any of the aspects, the anion is selected from Groups 1-7 of Table 5. In some embodiments of any of the aspects, the anion is selected from Group 9 of Table 5. In some embodiments of any of the aspects, the anion is selected from Group 10 of Table 5. In some embodiments of any of the aspects, the anion is selected from Groups 9-10 of Table 5.

In some embodiments of any of the aspects, the anion is geranic acid, octanoic acid, and/or citronellic acid. In some embodiments of any of the aspects, the anion is geranic acid. In some embodiments of any of the aspects, the anion is octanoic acid. In some embodiments of any of the aspects, the anion is citronellic acid. In some embodiments of any of the aspects, the anion comprises a carbon chain with an 8 carbon backbone. In some embodiments of any of the aspects, the anion is geranic acid, octenoic acid, octanoic acid, or citronellic acid. In some embodiments of any of the aspects, the anion is octenoic acid, octanoic acid, or citronellic acid.

As described herein, in selecting a cation to pair with the anion, the primary concern is that the cation not associate too closely with the anion—close association causes the anion to be retained on the initial side of the biological barrier. Choline and derivatives thereof are shown to be particularly well suited as IL cations for the types of anions described herein. Accordingly, the cation of an IL described herein can be a cation comprising a quaternary ammonium. A quarternary ammonion is a positively charged polyatomic ion of the structure NR₄⁺, each R independently being an alkyl group or an aryl group.

In some embodiments of any of the aspects, the cation has a molar mass equal to or greater than choline, e.g., a molar mass equal to or greater than 104.1708 g/mol. In some embodiments of any of the aspects, the cation has a molar mass greater than choline, e.g., a molar mass equal greater than 104.1708 g/mol.

In some embodiments of any of the aspects, each R group of the quarternary ammonium independently comprises an alkyl, alkane, alkene, or aryl. In some embodiments of any of the aspects, each R group of the quarternary ammonium independently comprises an alkyl, alkane, or alkene. In some embodiments of any of the aspects, each R group of the quarternary ammonium independently comprises an alkane or alkene. In some embodiments of any of the aspects, each R group of the quaternernary ammonium independently comprises a carbon chain of no more than 10 carbon atoms in length, e.g., no more than 10, 11, 12, 13, 14, 15, 16, 17, 18, 19, 20, 25, or 30 carbon atoms in length. In some embodiments of any of the aspects, each R group of the quaternernary ammonium independently comprises a carbon chain of no more than 12 carbon atoms in length. In some embodiments of any of the aspects, each R group of the quaternernary ammonium independently comprises a carbon chain of no more than 15 carbon atoms in length. In some embodiments of any of the aspects, each R group of the quaternernary ammonium independently comprises a carbon chain of no more than 20 carbon atoms in length.

In some embodiments of any of the aspects, each R group of the quaternernary ammonium independently comprises a carbon chain of no more than 10 carbon atoms, e.g., no more than 10, 11, 12, 13, 14, 15, 16, 17, 18, 19, 20, 25, or 30 carbon atoms. In some embodiments of any of the aspects, each R group of the quaternernary ammonium independently comprises a carbon chain of no more than 12 carbon atoms. In some embodiments of any of the aspects, each R group of the quaternernary ammonium independently comprises a carbon chain of no more than 15 carbon atoms. In some embodiments of any of the aspects, each R group of the quaternernary ammonium independently comprises a carbon chain of no more than 20 carbon atoms.

In some embodiments of any of the aspects, each R group of the quaternernary ammonium independently comprises an alkyl group of no more than 10 carbon atoms, e.g., no more than 10, 11, 12, 13, 14, 15, 16, 17, 18, 19, 20, 25, or 30 carbon atoms. In some embodiments of any of the aspects, each R group of the quaternernary ammonium independently comprises an alkyl group of no more than 12 carbon atoms. In some embodiments of any of the aspects, each R group of the quaternernary ammonium independently comprises an alkyl group of no more than 15 carbon atoms. In some embodiments of any of the aspects, each R group of the quaternernary ammonium independently comprises an alkyl group of no more than 20 carbon atoms.

In some embodiments of any of the aspects, each R group of the quaternernary ammonium independently comprises an alkane, alkene, aryl, heteroaryl, alkyl, or heteroalkyl. In some embodiments of any of the aspects, each R group of the quaternernary ammonium independently comprises an unsubstituted alkane, unsubstituted alkene, unsubstituted aryl, unsubstituted heteroaryl, unsubstituted alkyl, or unsubstituted heteroalkyl. In some embodiments of any of the aspects, each R group of the quaternernary ammonium independently an unsubstituted alkane. In some embodiments of any of the aspects, each R group of the quaternernary ammonium independently an unsubstituted alkene. In some embodiments of any of the aspects, each R group of the quaternernary ammonium independently comprises one or more substituent groups.

In some embodiments of any of the aspects, at least one R group of the quaternary ammonium comprises a hydroxy group. In some embodiments of any of the aspects, one R group of the quaternary ammonium comprises a hydroxy group. In some embodiments of any of the aspects, only one R group of the quaternary ammonium comprises a hydroxy group.

Exemplary, non-limiting cations can include choline and any of the cations designated C1-C7 which are defined by structure below.

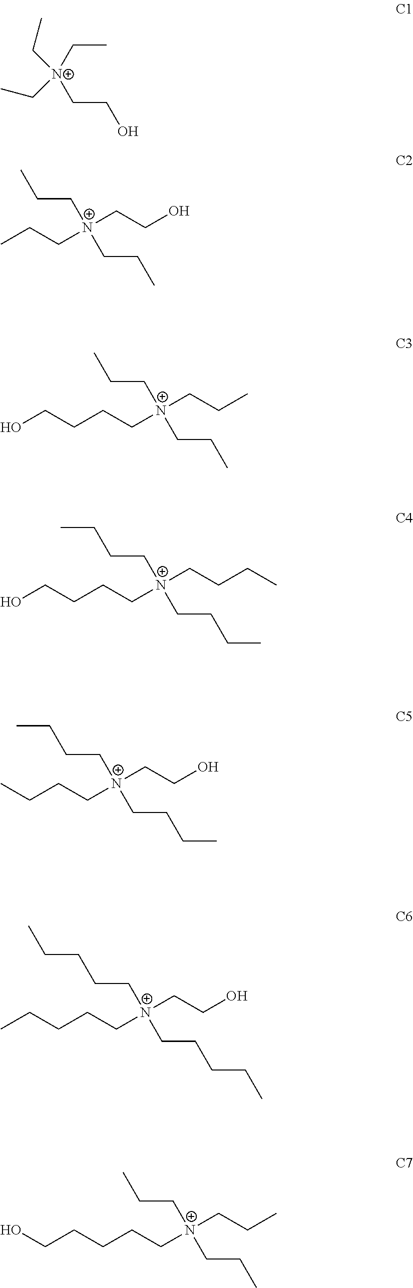

Further non-limiting examples of cations include the following: 1-(hydroxymethyl)-1-methylpyrrolidin-1-ium 1-(2-hydroxyethyl)-1-methylpyrrolidin-1-ium 1-ethyl-1-(3-hydroxypropyl)pyrrolidin-1-ium 1-(3-hydroxypropyl)-1-methylpyrrolidin-1-ium 1-(4-hydroxybutyl)-1-methylpyrrolidin-1-ium 1-ethyl-1-(4-hydroxybutyl)pyrrolidin-1-ium 1-(4-hydroxybutyl)-1-propylpyrrolidin-1-ium 1-(5-hydroxypentyl)-1-propylpyrrolidin-1-ium 1-ethyl-1-(5-hydroxypentyl)pyrrolidin-1-ium 1-(5-hydroxypentyl)-1-methylpyrrolidin-1-ium 1-(hydroxymethyl)-1-methylpiperidin-1-ium 1-(2-hydroxyethyl)-1-methylpiperidin-1-ium 1-ethyl-1-(2-hydroxyethyl)piperidin-1-ium 1-ethyl-1-(3-hydroxypropyl)piperidin-1-ium 1-(3-hydroxypropyl)-1-propylpiperidin-1-ium 1-(3-hydroxypropyl)-1-methylpiperidin-1-ium 1-(4-hydroxybutyl)-1-methylpiperidin-1-ium 1-ethyl-1-(4-hydroxybutyl)piperidin-1-ium 1-(4-hydroxybutyl)-1-propylpiperidin-1-ium I-butyl-1-(5-hydroxypentyl)piperidin-1-ium 1-(5-hydroxyl)entyl)-1-propylpiperidin-1-ium I-ethyl-1-(5-hydroxypentyl)piperidin-1-ium 1-(5-hydroxyl)entyl)-1-methylpiperidin-1-ium 3-ethyl-1-methyl-1H-imidazol-3-ium 1-methyl-3-propyl-1H-imidazol-3-ium 3-butyl-1-methyl-1H-imidazol-3-ium 1-methyl-3-pentyl-1H-imidazol-3-ium 1,2-dimethyl-3-pentyl-1H-imidazol-3-ium 3-butyl-1,2-dimethyl-1H-imidazol-3-ium 1,2-dimethyl-3-propyl-1H-imidazol-3-ium 3-(hydroxymethyl)-1,2-dimethyl-1H-imidazol-3-ium 3-(2-hydroxyethyl)-1,2-dimethyl-1H-imidazol-3-ium 3-(3-hydroxypropyl)-1,2-dimethyl-1H-imidazol-3-ium 3-(4-hydroxybutyl)-1,2-dimethyl-1H-imidazol-3-ium 3-(5-hydroxypentyl)-1,2-dimethyl-1H-imidazol-3-ium 3-(5-hydroxypentyl)-1-methyl-1H-imidazol-3-ium 3-(4-hydroxybutyl)-1-methyl-1H-imidazol-3-ium 3-(3-hydroxypropyl)-1-methyl-1H-imidazol-3-ium 3-(2-hydroxyethyl)-1-methyl-1H-imidazol-3-ium 3-(hydroxymethyl)-1,2,4,5-tetramethyl-1H-imidazol-3-ium 3-(2-hydroxyethyl)-1,2,4,5-tetramethyl-1H-imidazol-3-ium 3-(3-hydroxypropyl)-1,2,4,5-tetramethyl-1H-imidazol-3-ium 3-(4-hydroxybutyl)-1,2,4,5-tetramethyl-1H-imidazol-3-ium 3-(5-hydroxypentyl)-1,2,4,5-tetramethyl-1H-imidazol-3-ium 1-(5-hydroxypentyl)pyridin-1-ium 1-(4-hydroxybutyl)pyridin-1-ium 1-(3-hydroxypropyl)pyridin-1-ium 1-(2-hydroxyethyl)pyridin-1-ium 1-(hydroxymethyl)pyridin-1-ium 1-hydroxypyridin-1-ium (hydroxymethyl)trimethylphosphonium triethyl(hydroxymethyl)phosphonium triethyl(2-hydroxyethyl)phosphonium (2-hydroxyethyl)tripropylphosphonium (3-hydroxypropyl)tripropylphosphonium tributyl(3-hydroxypropyl)phosphonium (3-hydroxypropyl)tripentylphosphonium (4-hydroxybutyl)tripentylphosphonium (5-hydroxypentyl)tripentylphosphonium

In some embodiments of any of the aspects, the cation is choline, C1, C6, and/or C7. In some embodiments of any of the aspects, the cation is C1, C6, and/or C7.

In some embodiments of any of the aspects, the cation is selected from choline, C1, C6, and/or C7 and the anion is citronellic acid. In some embodiments of any of the aspects, the cation is choline and the anion is citronellic acid. In some embodiments of any of the aspects, the cation is C1 and the anion is citronellic acid. In some embodiments of any of the aspects, the cation is C6 and the anion is citronellic acid. In some embodiments of any of the aspects, the cation is C7 and the anion is citronellic acid.

In some embodiments of any of the aspects, the cation is selected from C1, C6, and/or C7 and the anion is geranic acid. In some embodiments of any of the aspects, the cation is C1 and the anion is geranic acid. In some embodiments of any of the aspects, the cation is C6 and the anion is geranic acid. In some embodiments of any of the aspects, the cation is C7 and the anion is geranic acid.

In some embodiments of any of the aspects, the cation is selected from choline, C1, C6, and/or C7 and the anion is octanoic acid. In some embodiments of any of the aspects, the cation is choline and the anion is octanoic acid. In some embodiments of any of the aspects, the cation is C1 and the anion is octanoic acid. In some embodiments of any of the aspects, the cation is C6 and the anion is ocatanoic acid. In some embodiments of any of the aspects, the cation is C7 and the anion is ocatanoic acid.

In some embodiments of any of the aspects, the cation is selected from choline, C1, C6, and C7 and the anion is selected from citronellic acid, octanoic acid, and octenoic acid. In some embodiments of any of the aspects, the cation is choline and the anion is selected from citronellic acid, octanoic acid, and octenoic acid. In some embodiments of any of the aspects, the ionic liquid is choline: citronellic acid, choline: octanoic acid, or choline: octenoic acid.

In some embodiments of any of the aspects, octenoic acid is 2-octenoic acid.

Non-limiting, exemplary combinations of cation and anions are provided in Table 6 below.

TABLE 6

| | Choline | C1 | C2 | C3 | C4 | C5 | C6 | C7 |
|---|---|---|---|---|---|---|---|---|
| Group 1 | | | | | | | | |
| Geranic Acid | | X | X | X | X | X | X | X |
| Citronellic Acid | X | X | X | X | X | X | X | X |
| Octenoic Acid | X | X | X | X | X | X | X | X |
| Decenoic Acid | X | X | X | X | X | X | X | X |
| (9Z)-octadec-9-enoic acid | X | X | X | X | X | X | X | X |
| Group 2 | | | | | | | | |
| Octanoic Acid | X | X | X | X | X | X | X | X |
| Decanoic Acid | X | X | X | X | X | X | X | X |
| (9Z, 12Z)-octadeca-9, | X | X | X | X | X | X | X | X |
| 12-dienoic acid | | | | | | | | |
| (R)-5-(1,2-dithiolan-3- | X | X | X | X | X | X | X | X |
| yl)pentanoic acid | | | | | | | | |
| Group 3 | | | | | | | | |
| Hexenoic Acid | X | X | X | X | X | X | X | X |
| Group 4 | | | | | | | | |
| Hexanoic Acid | X | X | X | X | X | X | X | X |
| 3-methylbutanoic acid | X | X | X | X | X | X | X | X |
| Nonanedioic Acid | X | X | X | X | X | X | X | X |
| Pentanoic acid | X | X | X | X | X | X | X | X |
| Group 5 | | | | | | | | |
| 2-hydroxyoctanoic acid | X | X | X | X | X | X | X | X |
| (E)-3-(4-hydroxy-3-methoxy- | X | X | X | X | X | X | X | X |
| phenyl)prop-2-enoic acid | | | | | | | | |
| Group 6 | | | | | | | | |
| 2-ethylhexyl sulfate | X | X | X | X | X | X | X | X |
| 2-(dimethylamino)ethanol | X | X | X | X | X | X | X | X |
| Group 7 | | | | | | | | |
| 8-hydroxycapric acid | X | X | X | X | X | X | X | X |
| 2-methylpropanoic acid | X | X | X | X | X | X | X | X |
| Ascorbic Acid | X | X | X | X | X | X | X | X |
| Butanoic acid | X | X | X | X | X | X | X | X |
| Salicylic Acid | X | X | X | X | X | X | X | X |
| Group 8 | | | | | | | | |
| Hydroxyl(phenyl)acetic acid | X | X | X | X | X | X | X | X |
| Glutaric Acid | X | X | X | X | X | X | X | X |
| Adipic acid | X | X | X | X | X | X | X | X |

In some embodiments of any of the aspects, the ionic liquid is not CAGE (Choline And GEranate). In some embodiments of any of the aspects, the cation of the ionic liquid is not choline. In some embodiments of any of the aspects, the anion of the ionic liquid is not geranate or geranic acid.

As demonstrated herein, the number of cross-peaks as measured by Nuclear Overhauser Effect SpectroscopY (NOESY) for a given IL correlates with its performance in drug delivery, with fewer cross peaks indicating better drug delivery performance. Fundamentally, the number of cross peaks indicates the intramolecular interactions between the ions that are mediated by protons. That is, each in-phase peak (the same color as the 1D diagonal line) indicates that molecules are within 5 nm of each other as an average across the liquid. In some embodiments of any of the aspects, the method described herein further comprises a step of measuring the number of cross-peaks of one or more ILs by NOESY. In some embodiments of any of the aspects, the ionic liquid described herein has less than 20 cross peaks as measured by Nuclear Overhauser Effect SpectroscopY (NOESY), e.g., less than 20, less than 15, less than 10, less than 9, less than 8, less than 7, less than 6, or less than 5. In some embodiments of any of the aspects, the ionic liquid described herein has less than 10 cross peaks as measured by Nuclear Overhauser Effect SpectroscopY (NOESY). In some embodiments of any of the aspects, the ionic liquid described herein has less than 5 cross peaks as measured by Nuclear Overhauser Effect SpectroscopY (NOESY). Methods of performing NOESY are known in the art and described in the Examples herein.

In one aspect of any of the embodiments, provided herein is a method of designing, selecting, and/or identifying an ionic liquid. The ionic liquid can be designed, selected, and/or identified for administration, administration by a particular route (e.g., transdermally, to a mucus membrane, orally, subcutaneously, intradermally, parenterally, intratumorally, or intravenously), and/or for delivery or administration of one or more active compounds. As described herein, ionic liquids are most advantageous for administration, administration by a particular route (e.g., transdermally, to a mucus membrane, orally, subcutaneously, intradermally, parenterally, intratumorally, or intravenously), and/or for delivery or administration of one or more active compounds when the inter-ionic interactions of the cation and anion are reduced. Accordingly, a cation and anion pair can be selected which minimizes the inter-ionic interactions. For example, the pair can be selected to minimize the interactions below a threshold provided herein, to minimize the interactions below a reference level, and/or to provide the most minimization of any pairwise combination of cation and anion of cations and anions from a pool or cations and anions.

Accordingly, in one aspect of any of the embodiments, provided herein is a method of designing and/or identifying an ionic liquid comprising two ions, wherein one ion is a cation and the other ion is an anion, the method comprising:

in a first option:

a. selecting one of the two ions of the ionic liquid; and

b. selecting the other ion to minimize inter-ionic interactions;

in a second option:

a. selecting the cation; and

b. selecting the anion to minimize inter-ionic interactions; or

in a third option:
a. selecting the anion; and
b. selecting the cation to minimize inter-ionic interactions.

In one aspect of any of the embodiments, provided herein is a method of designing and/or identifying an ionic liquid comprising two ions, wherein one ion is a cation and the other ion is an anion, from a pool of candidate cations and a pool of candidate anions, the method comprising: in a first option:
a. selecting one of the two ions of the ionic liquid from the pool of candidate ions; and
b. selecting from the other pool of candidate ions the ion which most minimizes inter-ionic interactions with the ion selected in step a;
   in a second option:
a. selecting the cation from the pool of candidate cations;
b. selecting from the pool of candidate anions the anion which most minimizes inter-ionic interactions with the cation selected in step a or
   in a third option:
a. selecting the cation from the pool of candidate anions;
b. selecting from the pool of candidate cations the anion which most minimizes inter-ionic interactions with the anion selected in step a.

In some embodiments of any of the aspects, an ionic liquid with minimized inter-ionic interactions has less than 20 cross peaks as measured by Nuclear Overhauser Effect SpectroscopY (NOESY), e.g., less than 20, less than 15, less than 10, less than 9, less than 8, less than 7, less than 6, or less than 5. In some embodiments of any of the aspects, an ionic liquid with minimized inter-ionic interactions has less than 10 cross peaks as measured by Nuclear Overhauser Effect SpectroscopY (NOESY). In some embodiments of any of the aspects, an ionic liquid with minimized inter-ionic interactions has less than 5 cross peaks as measured by Nuclear Overhauser Effect SpectroscopY (NOESY).

In some embodiments of any of the aspects, the IL is at a concentration of at least 0.01% w/v. In some embodiments of any of the aspects, the IL is at a concentration of at least 0.05% w/v. In some embodiments of any of the aspects, the IL is at a concentration of at least 0.1% w/v. In some embodiments of any of the aspects, the IL is at a concentration of at least 0.2% w/v, at least 0.3% w/v, at least 0.4% w/v, at least 0.5% w/v, at least 1% w/v or greater. In some embodiments of any of the aspects, the IL is at a concentration of from about 0.01% w/v to about 1% w/v. In some embodiments of any of the aspects, the IL is at a concentration of from 0.01% w/v to 1% w/v. In some embodiments of any of the aspects, the IL is at a concentration of from about 0.05% w/v to about 0.5% w/v. In some embodiments of any of the aspects, the IL is at a concentration of from 0.05% w/v to 0.5% w/v.

In some embodiments of any of the aspects, the IL is at a concentration of at least 25% w/w. In some embodiments of any of the aspects, the IL is at a concentration of at least 25% w/w in water. In some embodiments of any of the aspects, the IL is at a concentration of at least 25% w/w in saline or a physiologically compatible buffer.

In some embodiments of any of the aspects, the IL is at a concentration of from about 5% w/w to about 75% w/w. In some embodiments of any of the aspects, the IL is at a concentration of from 5% w/w to 75% w/w. In some embodiments of any of the aspects, the IL is at a concentration of from about 5% w/w to about 75% w/w in water, saline or a physiologically compatible buffer. In some embodiments of any of the aspects, the IL is at a concentration of from 5% w/w to 75% w/w in water, saline or a physiologically compatible buffer.

In some embodiments of any of the aspects, the IL is at a concentration of at least about 0.1% w/w. In some embodiments of any of the aspects, the IL is at a concentration of at least 0.1% w/w. In some embodiments of any of the aspects, the IL is at a concentration of from about 10% w/w to about 70% w/w. In some embodiments of any of the aspects, the IL is at a concentration of from 10% w/w to 70% w/w. In some embodiments of any of the aspects, the IL is at a concentration of from about 30% w/w to about 50% w/w. In some embodiments of any of the aspects, the IL is at a concentration of from 30% w/w to 40% w/w. In some embodiments of any of the aspects, the IL is at a concentration of from about 30% w/w to about 50% w/w. In some embodiments of any of the aspects, the IL is at a concentration of from 30% w/w to 40% w/w.

In some embodiments of any of the aspects, the % w/w concentration of the IL is % w/w concentration in water, saline, or a physiologically compatible buffer.

In some embodiments of any of the aspects, the IL is 100% by w/w or w/v.

In some embodiments, the IL is an anhydrous salt, e.g., an ionic liquid not diluted or dissolved in water. In some embodiments, the IL is provided as an aqueous solution.

In some embodiments of any of the aspects, the IL is at a concentration of at least 25% w/w and has a ratio of cation:anion of at least 1:3. In some embodiments of any of the aspects, the IL is at a concentration of at least 25% w/w in water and has a ratio of cation:anion of at least 1:3. In some embodiments of any of the aspects, the IL is at a concentration of at least 25% w/w and has a ratio of cation:anion of 1:3 or 1:4. In some embodiments of any of the aspects, the IL is at a concentration of at least 25% w/w in water and has a ratio of cation:anion of 1:3 or 1:4. In some embodiments of any of the aspects, the IL is a gel, or a shear-thinning Newtonian gel.

In some embodiments of any of the aspects, the IL has a ratio of cation:anion of from about 10:1 to about 1:10. In some embodiments of any of the aspects, the IL has a ratio of cation:anion of from 10:1 to 1:10. In some embodiments of any of the aspects, the IL has a ratio of cation:anion of from about 5:1 to about 1:5. In some embodiments of any of the aspects, the IL has a ratio of cation:anion of from 5:1 to 1:5. In some embodiments of any of the aspects, the IL has a ratio of cation:anion of from about 2:1 to about 1:4. In some embodiments of any of the aspects, the IL has a ratio of cation:anion of from 2:1 to 1:4. In some embodiments of any of the aspects, the IL has a ratio of cation:anion of from about 2:1 to about 1:10. In some embodiments of any of the aspects, the IL has a ratio of cation:anion of from 2:1 to 1:10. In some embodiments of any of the aspects, the IL has a ratio of cation:anion such that there is a greater amount of anion, e.g., a ratio of less than 1:1. In some embodiments of any of the aspects, the IL has a ratio of cation:anion such that there is an excess of anion. In some embodiments of any of the aspects, the IL has a ratio of cation:anion of from about 1:1 to about 1:10. In some embodiments of any of the aspects, the IL has a ratio of cation:anion of from 1:1 to 1:10. In some embodiments of any of the aspects, the IL has a ratio of cation:anion of from about 1:1 to about 1:4. In some embodiments of any of the aspects, the IL has a ratio of cation:anion of from 1:1 to 1:4. In some embodiments of any of the aspects, the IL has a ratio of cation:anion of from about 1:1 to about 1:3. In some embodiments of any of the aspects, the IL has a ratio of cation:anion of from 1:1 to 1:3. In some embodiments of any of the aspects, the IL has a ratio of cation:anion of from about 1:1 to about 1:2. In some embodiments of any of the aspects, the IL has a ratio of cation:anion of from 1:1 to 1:2. In some embodiments of any of the aspects, the IL has a ratio of cation:anion of about 1:1, 1:2, 1:3, or 1:4. In some embodiments of any of the aspects, the IL has a ratio of cation:anion of 1:1, 1:2, 1:3, or 1:4. Without wishing to be constrained by theory, compositions with higher amounts of anion relative to cation display greater hydrophobicity.

In some embodiments of any of the aspects, e.g., when one or more nucleic acid molecules are provided in combination with the IL, the ratio of cation:anion is greater than 1:1, e.g., greater than 1:2, from about 1:2 to about 1:4, or from 1:2 to 1:4.

In some embodiments of any of the aspects, the IL is at a concentration of at least 20 mM. In some embodiments of any of the aspects, the IL is at a concentration of at least about 20 mM. In some embodiments of any of the aspects, the IL is at a concentration of at least 25 mM. In some embodiments of any of the aspects, the IL is at a concentration of at least about 25 mM. In some embodiments of any of the aspects, the IL is at a concentration of at least 50 mM. In some embodiments of any of the aspects, the IL is at a concentration of at least about 50 mM. In some embodiments of any of the aspects, the IL is at a concentration of at least 100 mM, 500 mM, 1 M, 2 M, 3 M or greater. In some embodiments of any of the aspects, the IL is at a concentration of at least about 100 mM, 500 mM, 1 M, 2 M, 3 M or greater.

In some embodiments of any of the aspects, the IL is at a concentration of from about 50 mM to about 4 M. In some embodiments of any of the aspects, the IL is at a concentration of from 50 mM to 4 M. In some embodiments of any of the aspects, the IL is at a concentration of from about 500 mM to about 4 M. In some embodiments of any of the aspects, the IL is at a concentration of from 500 mM to 4 M. In some embodiments of any of the aspects, the IL is at a concentration of from about 1 M to about 4 M. In some embodiments of any of the aspects, the IL is at a concentration of from 1 M to 4 M. In some embodiments of any of the aspects, the IL is at a concentration of from about 2 M to about 4 M. In some embodiments of any of the aspects, the IL is at a concentration of from 2 M to 4 M.

In some embodiments of any of the aspects, the IL concentration in the composition or formulation is about 0.1 mM to 20 mM. In some embodiments of any of the aspects, the IL concentration in the composition or formulation is about 0.5 mM to 20 mM, 0.5 mM to 18 mM, 0.5 mM to 16 mM, 0.5 mM to 14 mM, 0.5 mM to 12 mM, 0.5 mM to 10 mM, 0.5 mM to 8 mM, 1 mM to 20 mM, 1 mM to 18 mM, 1 mM to 16 mM, 1 mM to 14 mM, 1 mM to 12 mM, 1 mM to 10 mM, 1 mM to 8 mM, 2 mM to 20 mM, 2 mM to 18 mM, 2 mM to 16 mM, 2 mM to 14 mM, 2 mM to 12 mM, 2 mM to 10 mM, 2 mM to 8 mM, 4 mM to 20 mM, 4 mM to 18 mM, 4 mM to 16 mM, 4 mM to 14 mM, 4 mM to 12 mM, 4 mM to 10 mM, 4 mM to 8 mM, 6 mM to 20 mM, 6 mM to 18 mM, 6 mM to 14 mM, 6 mM to 12 mM, 6 mM to 10 mM, 6 mM to 8 mM, 8 mM to 20 mM, 8 mM to 18 mM, 8 mM to 16 mM, 8 mM to 14 mM, 8 mM to 12 mM, 8 mM to 10 mM, 10 mM to 20 mM, 10 mM to 18 mM, 10 mM to 16 mM, 10 mM to 14 mM, 10 mM to 12 mM, 12 mM to 20 mM, 12 mM to 18 mM, 12 mM to 16 mM, 12 mM to 14 mM, 14 mM to 20 mM, 14 mM to 18 mM, 14 mM to 16 mM, 16 mM to 20 mM, 16 mM to 18 mM, or 18 mM to 20 mM. In some embodiments of any of the aspects, the IL concentration in the composition or formulation is about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM or about 20 mM.

It is specifically contemplated that a composition or combination described herein can comprise one, two, three, or more of any of the types of components described herein. For example, a composition can comprise a mixture, solution, combination, or emulsion of two or more different ionic liquids, and/or a mixture, solution, combination, or emulsion of two or more different non-ionic surfactants, and/or a mixture, solution, combination, or emulsion of two or more different active compounds.

As used herein, “in combination with” refers to two or more substances being present in the same formulation in any molecular or physical arrangement, e.g, in an admixture, in a solution, in a mixture, in a suspension, in a colloid, in an emulsion. The formulation can be a homogeneous or heterogenous mixture. In some embodiments of any of the aspects, the active compound(s) can be comprised by a superstructure, e.g., nanoparticles, liposomes, vectors, cells, scaffolds, or the like, said superstructure is which in solution, mixture, admixture, suspension, etc., with the IL.

As used herein, an “active compound” or “active agent” is any agent which will exert an effect on a target cell or organism. The terms “compound” and “agent” refer to any entity which is normally not present or not present at the levels being administered and/or provided to a cell, tissue or subject. An agent can be selected from a group comprising: chemicals; small organic or inorganic molecules; signaling molecules; nucleic acid sequences; nucleic acid analogues; proteins; peptides; enzymes; aptamers; peptidomimetic, peptide derivative, peptide analogs, antibodies; intrabodies; biological macromolecules, extracts made from biological materials such as bacteria, plants, fungi, or animal cells or tissues; naturally occurring or synthetic compositions or functional fragments thereof. In some embodiments, the agent is any chemical, entity or moiety, including without limitation synthetic and naturally-occurring non-proteinaceous entities. Agents can be known to have a desired activity and/or property, or can be selected from a library of diverse compounds. Non-limiting examples of active compounds contemplated for use in the methods described herein include small molecules, polypeptides, nucleic acids, chemotherapies/chemotherapeutic compounds, antibodies, antibody reagents, vaccines, a GLP-1 polypeptide or mimetic/analog thereof, insulin, acarbose, or ruxolitinib.

A nucleic acid molecule, as described herein, can be a vector, an expression vector, an inhibitory nucleic acid, an aptamer, a template molecule or cassette (e.g., for gene editing), or a targeting molecule (e.g., for CRISPR-Cas technologies), or any other nucleic acid molecule that one wishes to deliver to a cell. The nucleic acid molecule can be RNA, DNA, or synthetic or modified versions thereof.

In one aspect of any of the embodiments, described herein is a method of delivering a nucleic acid molecule to a cell, the method comprising contacting the cell with the nucleic acid molecule in combination with an IL as described herein. In some embodiments of any of the aspects, the cell is a cell in a subject and the contacting step comprises administering the nucleic acid molecule in combination with the IL to the subject. In some embodiments of any of the aspects, the cell is in vitro, in vivo, or ex vivo. In some embodiments of any of the aspects, the cell is eurkaryotic. In some embodiments of any of the aspects, the cell is mammalian. In some embodiments of any of the aspects, the cell is an epithelial cell, e.g, an intestinal epithelial cell.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

In some embodiments of any of the aspects, the active compound can be a therapeutic compound or drug, e.g., an agent or compound which is therapeutically effective for the treatment of at least one condition in a subject. Therapeutic compounds are known in the art for a variety of conditions, see, e.g., the database available on the world wide web at drugs.com or the catalog of FDA-approved compounds available on the world wide web at catalog.data.gov/dataset/drugsfda-database; each of which is incorporated by reference herein in its entirety.

As used herein the term “chemotherapeutic agent” refers to any chemical or biological agent with therapeutic usefulness in the treatment of diseases characterized by abnormal cell growth. Such diseases include tumors, neoplasms and cancer as well as diseases characterized by hyperplastic growth. These agents can function to inhibit a cellular activity upon which the cancer cell depends for continued proliferation. In some aspect of all the embodiments, a chemotherapeutic agent is a cell cycle inhibitor or a cell division inhibitor. Categories of chemotherapeutic agents that are useful in the methods of the invention include alkylating/alkaloid agents, antimetabolites, hormones or hormone analogs, and miscellaneous antineoplastic drugs. Most of these agents are directly or indirectly toxic to cancer cells. In one embodiment, a chemotherapeutic agent is a radioactive molecule.

In some embodiments of any of the aspects, the active compound is a hydrophobic molecule, e.g., estradiol, testosterone, corticosterone, paclitaxel, doxorubicin, cisplatin, and/or camptothecin. In some embodiments of any of the aspects, the active compound is a hydrophilic molecule.

In some embodiments of any of the aspects, the active compound is an antibody or antibody reagent. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

In some embodiments of any of the aspects, the active compound has a molecular weight of greater than about 450. In some embodiments of any of the aspects, the active compound has a molecular weight of greater than about 500. In some embodiments of any of the aspects, the active compound has a molecular weight of greater than 450, e.g., greater than 450, greater than 500, greater than 550, greater than 600, greater than 1000 or more. In some embodiments of any of the aspects, the active compound is polar.

In some embodiments of any of the aspects, a composition or combination as described herein, comprising at least one IL and optionally an active compound can be formulated as an oral, subcutaneous, intravenous, intradermal, or parenteral formulation. In some embodiments of any of the aspects, an oral formulation can be a degradable capsule comprising the composition comprising the at least one IL and optionally, an active compound.

In some embodiments of any of the aspects, described herein is a composition comprising at least one IL as described herein and at least one active compound. In some embodiments of any of the aspects, described herein is a composition consisting essentially of at least one IL as described herein and at least one active compound. In some embodiments of any of the aspects, described herein is a composition consisting of at least one IL as described herein and at least one active compound. In some embodiments of any of the aspects, the composition comprising at least one IL as described herein and at least one active compound is administered as a monotherapy, e.g., another treatment for the condition is not administered to the subject.

In one aspect of any of the embodiments, described herein is a pharmaceutical composition comprising at least one active compound in combination with at least one IL as described herein. In some embodiments, the pharmaceutical composition comprises the at least one IL and the one or more active compounds as described herein. In some embodiments, the pharmaceutical composition consists essentially of the at least one IL and the one or more active compounds as described herein. In some embodiments, the pharmaceutical composition consists of the at least one IL and the one or more active compounds as described herein. In some embodiments, the pharmaceutical composition consists essentially of an aqeuous solution of the at least one IL and the one or more active compounds as described herein. In some embodiments, the pharmaceutical composition consists of an aqeuous solution of the at least one IL and the one or more active compounds as described herein.

The compositions, formulations, and combinations described herein can comprise at least one IL as described herein, e.g., one IL, two ILs, three ILs, or more. In some embodiments of any of the aspects, a composition, formulation, or combination as described herein can comprise at least one IL as described herein and CAGE (Choline And GEranate).

In some embodiments of any of the aspects, the at least one active compound and the at least one ionic liquid are further in combination with at least one non-ionic surfactant. As used herein, “non-ionic surfactant” refers to a surfactant which lacks a net ionic charge and does not dissociate to an appreciable extent in aqueous media. The properties of non-ionic surfactants are largely dependent upon the proportions of the hydrophilic and hydrophobic groups in the molecule. Hydrophilic groups include the oxyethylene group (—OCH2 CH2-) and the hydroxy group. By varying the number of these groups in a hydrophobic molecule, such as a fatty acid, substances are obtained which range from strongly hydrophobic and water insoluble compounds, such as glyceryl monostearate, to strongly hydrophilic and water-soluble compounds, such as the macrogols. Between these two extremes types include those in which the proportions of the hydrophilic and hydrophobic groups are more evenly balanced, such as the macrogol esters and ethers and sorbitan derivatives. Suitable non-ionic surfactants may be found in Martindale, The Extra Pharmacopoeia, 28th Edition, 1982, The Pharmaceutical Press, London, Great Britain, pp. 370 to 379. Non-limiting examples of non-ionic surfactants include polysorbates, a Tween™, block copolymers of ethylene oxide and propylene oxide, glycol and glyceryl esters of fatty acids and their derivatives, polyoxyethylene esters of fatty acids (macrogol esters), polyoxyethylene ethers of fatty acids and their derivatives (macrogol ethers), polyvinyl alcohols, and sorbitan esters, sorbitan monoesters, ethers formed from fatty alcohols and polyethylene glycol, polyoxyethylene-polypropylene glycol, alkyl polyglycoside, Cetomacrogol 1000, cetostearyl alcohol, cetyl alcohol, cocamide DEA, cocamide MEA, decyl glucoside, decyl polyglucose, glycerol monostearate, IGEPAL CA-630, isoceteth-20, lauryl glucoside, maltosides, monolaurin, mycosubtilin, Nonidet P-40, nonoxynol-9, nonoxynols, NP-40, octaethylene glycol monododecyl ether, N-Octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, sorbitan, sorbitan monolaurate, sorbitan monostearate, sorbitan tristearate, stearyl alcohol, surfactin, Triton X-100, and the like. In some embodiments of any of the aspects, the at least one non-ionic surfactant has a neutral hydrophilic head group.

As used herein, “polysorbate” refers to a surfactant derived from ethoxylated sorbitan (a derivative of sorbitol) esterified with fatty acids. Common brand names for polysorbates include Scattics™, Alkest™, Canarcel™, and Tween™. Exemplary polysorbates include polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate).

In some embodiments of any of the aspects, the at least one non-ionic surfactant (e.g., at least one polysorbate) is present at a concentration of about 0.1% to about 50% w/v. In some embodiments of any of the aspects, the at least one non-ionic surfactant (e.g., at least one polysorbate) is present at a concentration of 0.1% to 50% w/v. In some embodiments of any of the aspects, the at least one non-ionic surfactant (e.g., at least one polysorbate) is present at a concentration of about 1% to about 5% w/v. In some embodiments of any of the aspects, the at least one non-ionic surfactant (e.g., at least one polysorbate) is present at a concentration of 1% to 5% w/v. In some embodiments of any of the aspects, the at least one non-ionic surfactant (e.g., at least one polysorbate) is present at a concentration of about 3% to about 10% w/v. In some embodiments of any of the aspects, the at least one non-ionic surfactant (e.g., at least one polysorbate) is present at a concentration of 3% to 10% w/v. In some embodiments of any of the aspects, the at least one non-ionic surfactant (e.g., at least one polysorbate) is present at a concentration of less than about 5% w/v. In some embodiments of any of the aspects, the at least one non-ionic surfactant (e.g., at least one polysorbate) is present at a concentration of less than 5% w/v.

In some embodiments of any of the aspects, the combination of the at least one active compound and at least one IL as described herein is provided in one or more nanoparticles. In some embodiments of any of the aspects, the combination of the at least one active compound and at least one IL as described herein comprises nanoparticles comprising the active compound, the nanoparticles in solution or suspension in a composition comprising at least one IL as described herein.

In some embodiments of any of the aspects, a composition as described herein, e.g., a composition comprising at least one IL and an active compound, can further comprise a pharmaceutically acceptable carrier. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present disclosure can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques. Suitable pharmaceutical carriers are described in Remington's Pharmaceutical Sciences, A. Osol, a standard reference text in this field of art. For example, a parenteral composition suitable for administration by injection is prepared by dissolving 1.5% by weight of active ingredient in 0.9% sodium chloride solution.

The term “carrier” in the context of a pharmaceutical carrier refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active compound. The term “pharmaceutically acceptable carrier” excludes tissue culture medium.

In some embodiments of any of the aspects, a composition as described herein, e.g, a composition comprising at least one IL as described herein and an active compound, can be formulated as an oral, subcutaneous, intravenous, intradermal, or parenteral formulation. In some embodiments of any of the aspects, an oral formulation can be a degradable capsule comprising the composition described herein, e.g., a composition comprising at least one IL as described herein and an active compound.

In some embodiments of any of the aspects described herein, the biological activity of the active compound is improved or stabilized as compared to the activity in the absence of the at least one IL. In some embodiments of any of the aspects described herein, the greatly enhances permeation of the active compound across the skin compared to a control where the at least one IL is absent.

In one aspect of any of the embodiments, described herein is a method of administering at least active compound to a subject using a catheter wherein the catheter is coated with at least one IL as described herein. In one aspect of any of the embodiments, described herein is a method of collecting a body fluid by placing the catheter into the body wherein the catheter is coated with at least one IL as described herein.

In one aspect of any of the embodiments, the composition or combination described herein is for a method of administering or delivering at least one active compound, e.g., for the treatment of a disease. In one aspect of any of the embodiments, described herein is a method of administering at least one active compound, the method comprising administering the active compound in combination with at least one IL as described herein. In one aspect of any of the embodiments, described herein is a method of treating a disease by administering at least one active compound, the method comprising administering the active compound in combination with at least one IL as described herein.

In some embodiments, the methods described herein relate to treating a subject having or diagnosed as having a condition with a composition as described herein, e.g, a comprising at least one IL and an active compound. Subjects having a condition, e.g., diabetes, can be identified by a physician using current methods of diagnosing diabetes. Symptoms and/or complications of diabetes which characterize these conditions and aid in diagnosis are well known in the art and include but are not limited to, weight loss, slow healing, polyuria, polydipsia, polyphagiam headaches, itchy skin, and fatigue. Tests that may aid in a diagnosis of, e.g. diabetes include, but are not limited to, blood tests (e.g., for fasting glucose levels). A family history of diabetes, or exposure to risk factors for diabetes (e.g. overweight) can also aid in determining if a subject is likely to have diabetes or in making a diagnosis of diabetes.

The compositions and methods described herein can be administered to a subject having or diagnosed as having a condition described herein. In some embodiments, the methods described herein comprise administering an effective amount of compositions described herein, e.g. a composition comprising at least one IL as described herein and an active compound, to a subject in order to alleviate a symptom of a condition described herein. As used herein, “alleviating a symptom” is ameliorating any marker or symptom associated with a condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, injection, or intratumoral administration. Administration can be local or systemic.

In some embodiments of any of the aspects, the administration is transdermal. In some embodiments of any of the aspects, the administration is transdermal, to a mucus membrane (e.g., to a nasal, oral, or vaginal membrane), oral, subcutaneous, intradermal, parenteral, intratumoral, or intravenous.

Oral administration can comprise providing tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Oral formulations can comprise discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of an ionic liquid as described herein and the at least one active compound, and may be prepared by methods of pharmacy well known to those skilled in the art. See generally, Remington: The Science and Practice of Pharmacy, 21st Ed., Lippincott, Williams, and Wilkins, Philadelphia Pa. (2005).

In one aspect of any of the embodiments, described herein is a method of delivery of at least one active compound by subcutaneous, intradermal or intravenous administration, the method comprising administering the active compound in combination with at least one IL as described herein. In some embodiments of any of the aspects, subcutaneous, intradermal or intravenous administration comprises administration via injection, catheter, port, or the like.

In one aspect of any of the embodiments, described herein is a method of parenteral delivery of at least one active compound, the method comprising parenterally administering the active compound in combination with at least one IL as described herein. In some embodiments, the parenteral administration comprises delivery to a tumor, e.g., a cancer tumor. In some embodiments of any of the aspects, the composition or combination described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a composition comprising an ionic liquid as described herein in combination with at least one active compound as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of an ingredient in a composition as disclosed herein can also be incorporated into the parenteral dosage forms of the disclosure, including conventional and controlled-release parenteral dosage forms.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. While as noted above herein, the compositions comprising an ionic liquid as described herein in combination with at least one active compound can obviate certain reasons for using a controlled-release formulation, it is contemplated herein that the methods and compositions can be utilized in controlled-release formulations in some embodiments. For example, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition comprising an ionic liquid as described herein in combination with at least one active compound can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active compound, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for blood glucose, among others. The dosage can be determined by a physician and adjusted, as necessary, to suit observed effects of the treatment.

As used herein, “diabetes” refers to diabetes mellitus, a metabolic disease characterized by a deficiency or absence of insulin secretion by the pancreas. As used throughout, “diabetes” includes Type 1, Type 2, Type 3, and Type 4 diabetes mellitus unless otherwise specified herein. The onset of diabetes is typically due to a combination of hereditary and environmental causes, resulting in abnormally high blood sugar levels (hyperglycemia). The two most common forms of diabetes are due to either a diminished production of insulin (in type 1), or diminished response by the body to insulin (in type 2 and gestational). Both lead to hyperglycemia, which largely causes the acute signs of diabetes: excessive urine production, resulting compensatory thirst and increased fluid intake, blurred vision, unexplained weight loss, lethargy, and changes in energy metabolism. Diabetes can cause many complications. Acute complications (hypoglycemia, ketoacidosis, or nonketotic hyperosmolar coma) may occur if the disease is not adequately controlled. Serious long-term complications (i.e. chronic side effects) include cardiovascular disease (doubled risk), chronic renal failure, retinal damage (which can lead to blindness), nerve damage (of several kinds), and microvascular damage, which may cause impotence and poor wound healing. Poor healing of wounds, particularly of the feet, can lead to gangrene, and possibly to amputation. In some embodiments, the diabetes can be Type 2 diabetes. Type 2 diabetes (non-insulin-dependent diabetes mellitus (NIDDM), or adult-onset diabetes) is a metabolic disorder that is primarily characterized by insulin resistance (diminished response by the body to insulin), relative insulin deficiency, and hyperglycemia. In some embodiments, a subject can be pre-diabetic, which can be characterized, for example, as having elevated fasting blood sugar or elevated post-prandial blood sugar.

Glucagon-Like Peptide-1(GLP-1), is known to reduce food intake and hunger feelings in humans and is an incretin derived from the transcription product of the proglucagon gene that contributes to glucose homeostasis. GLP-1 mimetics are currently being used in the treatment of Type 2 diabetes. Recent clinical trials have shown that these treatments not only improve glucose homeostasis but also succeed in inducing weight loss. As used herein. “GLP-1 polypeptide” refers to the various pre- and pro-peptides and cleavage products of GLP-1, e.g., for human: GLP-1(1-37) (SEQ ID NO: 2), GLP-1 (7-36) (SEQ ID NO: 3), and GLP-1 (7-37) (SEQ ID NO: 4). In some embodiments, a GLP-1 polypeptide can be GLP-1 (7-36) and/or GLP-1 (7-37) or the correlating polypeptides from a species other than human. Sequences for GLP-1 polypeptides are known in the art for a number of species, e.g. human GLP-1 (NCBI Gene ID: 2641) polypeptides (e.g., NCBI Ref Seq: NP_002045.1; SEQ ID NO: 1) and SEQ ID NOs: 2-4. In some embodiments, a pre or pro-peptide of GLP-1 can be used in the methods or compositions described herein, e.g., a glucagon preproprotein (e.g., SEQ ID NO: 1). Naturally-occurring alleles or variants of any of the polypeptides described herein are also specifically contemplated for use in the methods and compositions described herein.

SEQ ID NO: 1

| | |
|---|---|
| 1 | mksiyfvagl fvmlvqgswq rslqdteeks rsfsasqadp |
| | lsdpdqmned krhsqgtfts |
| 61 | dyskyldsrr aqdfvqwlmn tkrnrnniak rhdeferhae |
| | gtftsdvssy legqaakefi |
| 121 | awlvkgrgrr dfpeevaive elgrrhadgs fsdemntild |
| | nlaardfinw liqtkitdrk |

SEQ ID NO: 2
hdeferhae gtftsdvssy legqaakefi awlvkgrg
SEQ ID NO: 3
hae gtftsdvssy legqaakefi awlvkgr
SEQ ID NO: 4
hae gtftsdvssy legqaakefi awlvkgrg

Various GLP-1 mimetics are known in the art and used in the treatment of diabetes. GLP-1 mimetics (or analogues) can include exendin-4 (a Heloderma lizard polypeptide with homology to human GLP-1) and derivatives thereof, GLP-1 analogs modified to be DPP-IV resistant, or human GLP-1 polypeptides conjugated to various further agents, e.g., to extend the half-life. GLP-1 mimetics/analogues can include, e.g., exenatide, lixisenatide, dulaglutide, semaglutide, albiglutide, LY2189265, liraglutide, and taspoglutide. Examples of such molecules and further discussion of their manufacture and activity can be found in the art, e.g., Gupta. Indian J. Endocrinol Metab 17:413-421 (2013); Garber. Diabetes Treatments 41:S279-S284 (2018); US Patent Publication US2009/0181912; and International Patent Publication WO2011/080103, each of which is incorporated by reference herein in its entirety.

In some embodiments of any of the aspects, the active compound can be a chemotherapeutic agent or agent effective for the treatment of cancer. As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastases. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

In some embodiments of any of the aspects, the composition as described herein, e.g., a composition comprising at least one IL as described herein in combination with at least one active compound, is administered as a monotherapy, e.g., another treatment for the condition is not administered to the subject.

In some embodiments of any of the aspects, the methods described herein can further comprise administering a second agent and/or treatment to the subject, e.g. as part of a combinatorial therapy, either in the composition described herein, e.g., a composition comprising at least one IL as described herein in combination with at least one active compound, or as a separate formulation. For example, non-limiting examples of a second agent and/or treatment for treatment of cancer can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE.RTM. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb.RTM.); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In addition, the methods of treatment can further include the use of radiation or radiation therapy. Further, the methods of treatment can further include the use of surgical treatments.

Due to the excellent transdermal drug delivery characteristics of the ILs described herein, the compositions and combinations described herein are suitable for use with active compounds effective in treating skin diseases and alopoeica, e.g., alopoecia areta. Suitable active compounds for the treatment of alopoeica can include, e.g., corticosteroids (e.g., clobetasol or fluocinonide), minoxidil, Elocon (mometasone), irritants (e.g., anthralin or topical coal tar), and ciclosporin.

In certain embodiments, an effective dose of a composition described herein, e.g, a composition comprising at least one IL as described herein in combination with at least one active compound, can be administered to a patient once. In certain embodiments, an effective dose a composition described herein, e.g, a composition comprising at least one IL as described herein in combination with at least one active compound, can be administered to a patient repeatedly. For systemic administration, subjects can be administered a therapeutic amount of a composition described herein, e.g, a composition comprising at least one IL as described herein in combination with at least one active compound, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more. In some embodiments of any of the aspects, the at least one active compound is present in the combination at a dose of from about 1.0-20.0 mg/kg. In some embodiments of any of the aspects, the at least one active compound is present in the combination at a dose of from 1.0-20.0 mg/kg.

In some embodiments, the active compound is insulin and the concentration or dosage of insulin can be from about 1 U/kg to about 20 U/kg. In some embodiments, the active compound is insulin and the concentration or dosage of insulin can be from 1 U/kg to 20 U/kg. In some embodiments, the active compound is insulin and the concentration or dosage of insulin can be less than 20 U/kg. In some embodiments, the active compound is insulin and the concentration or dosage of insulin can be from about 2 U/kg to about 10 U/kg. In some embodiments, the active compound is insulin and the concentration or dosage of insulin can be from 2 U/kg to 10 U/kg. In some embodiments, the active compound is insulin and the concentration or dosage of insulin can be from about 2 U/kg to about 5 U/kg. In some embodiments, the active compound is insulin and the concentration or dosage of insulin can be from 2 U/kg to 5 U/kg. In some embodiments, the active compound is insulin and the concentration or dosage of insulin can be from about 5 U/kg to about 10 U/kg. In some embodiments, the active compound is insulin and the concentration or dosage of insulin can be from 5 U/kg to 10 U/kg. In some embodiments, the active compound is insulin and the concentration or dosage of insulin can be 2 U/kg, 5 U/kg, or 10 U/kg.

In one aspect of any of the embodiments, described herein is a method of treating a disease in a subject in need thereof by administering to the subject an active compound in combination with the at least one IL as described herein by into the affected tissue by injection. In some embodiments, the affected tissue is tissue comprising diseased cells. In some embodiments, the affected tissue is tissue displaying symptoms of the disease. Non-limiting examples of suitable affected tissues include tumor tissue, fat tissue, adipose tissue, or the like. In some embodiments of any of the aspects, suitable affected tissues include tumor tissue, fat tissue, adipose tissue, or the like. In some embodiments of any of the aspects, the disease is a disease arising from tissue growth, e.g., unwanted, aberrant, or pathological tissue growth. A disease arising from tissue growth can be any disease caused by or characterized by, a rate of tissue growth, location of tissue growth, or pattern/structure of tissue growth which differs from what is normal for that tissue type in a healthy subject. Non-limiting examples of such diseases are tumors, cancer, fat/obesity, and/or hyperplasia. In some embodiments of any of the aspects, such diseases are tumors, cancer, fat/obesity, and/or hyperplasia.

Enzyme inhibitors are a treatment option for a number of conditions, including diabetes, where, for example, insulin-degrading enzyme inhibitors, ACE inhibitors, and alpha-glucosidase inhibitors have all been explored as therapeutic approaches. Safe, effective enzyme inhibitors are therefore of interest in the treatment of a number of conditions. Without wishing to be bound by theory, it is contemplated herein that the ILs described herein can exhibit enzyme inhibition activity. Accordingly, in one aspect of any of the embodiments, described herein is a method of treating diabetes, ulcers, cancer, or fibrosis in a subject in need thereof, the method comprising administering to the subject a composition comprising at least one IL as described herein. In some embodiments, the composition does not comprise a further therapeutically active agent.

Fibrotic conditions benefit from the production and/or maintenance of the extracellular matrix by reducing the accumulation of scar tissue in favor of extracellular matrix. As used herein, “fibrosis” refers to the formation of fibrous tissue as a reparative or reactive process, rather than as a normal constituent of an organ or tissue. Fibrosis is characterized by fibroblast accumulation and collagen deposition in excess of normal deposition in any particular tissue. Fibrosis can occur as the result of inflammation, irritation, or healing. A subject in need of treatment for a fibrotic condition is any subject having, or diagnosed as having, or at risk of having a fibrotic condition. Non-limiting examples of fibrotic conditions include, but are not limited to pulmonary fibrosis; scarring; scarring of the skin; trauma; a wound; chronic wounds (e.g. as in diabetes patients), corneal defects; corneal ulceration; corneal wounds; diabetic ulcer; ulcer; sepsis; arthritis; idiopathic pulmonary fibrosis; cystic fibrosis; cirrhosis; endomyocardial fibrosis; mediastinal fibrosis; myelofibrosis; retroperitoneal fibrosis; progressive massive fibrosis; nephrogenic systemic fibrosis; Crohn's disease; keloid; scleroderma; systemic sclerosis; arthrofibrosis; adhesive capsulitis; lung fibrosis; liver fibrosis; kidney fibrosis; heart fibrosis; vascular fibrosis; skin fibrosis; eye fibrosis; bone marrow fibrosis; asthma; sarcoidosis; COPD; emphysema; nschistomasomiasis; cholangitis; diabetic nephropathy; lupus nephritis; postangioplasty arterial restenosis; atherosclerosis; burn scarring; hypertrophic scarring; nephrogenic fibrosing dermatopathy; postcataract surgery; proliferative vitreoretinopathy; Peyronie's disease; Duputren's contracture; dermatomyositis; and graft versus host disease.

As used herein, “ulcer” refers to a break or disruption of a bodily membrane. In some embodiments, the ulcer can be caused by inflammation and/or necrosis of the affected tissue. Ulcers can be skin ulcers (e.g., pressure ulcers, diabetic ulcers, ulcerative dermatitis, and the like), a corneal ulcer, an oral ulcer, a peptic ulcer, a venousucler, a stress ulcer, or ulcerative colitis.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active compound. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition described herein, e.g, a composition comprising at least one IL in combination with at least one active compound, can be administered over a period of time, such as over a 5 minute, 10 minute, 15 minute, 20 minute, or 25 minute period.

The dosage ranges for the administration of the compositions described herein, according to the methods described herein depend upon, for example, the form of the active compound, its potency, and the extent to which symptoms, markers, or indicators of a condition described herein are desired to be reduced, for example the percentage reduction desired for symptoms or markers. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition described in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of diabetes or cancer. When using an experimental animal model, efficacy of treatment is evidenced when a statistically significant change in a marker is observed.

In vitro and animal model assays are provided herein which allow the assessment of a given dose of a composition described herein, e.g, a composition comprising at least one IL in combination with at least one active compound. By way of non-limiting example, the effects of a dose of a composition comprising at least one IL in combination with insulin can be assessed by using the models described in the Examples herein.

The incidence of obesity is on the rise and existing treatments, such as diets, have notoriously low long-term success rates. Additional treatments and strategies for reducing obesity or reducing weight gain rates are of critical importance both for addressing obesity itself as well the number of conditions that are caused by or exacerbated by excess weight. Without wishing to be bound by theory, it is contemplated herein that the ILs as described herein can reduce the uptake of hydrophobic/lipophilic molecules in the intestine. Accordingly, provided herein are methods of treating obesity and/or reducing weight/weight gain by administering at least one IL as described herein to a subject in need of such. In some embodiments of any of the aspects, a subject treated in accordance with the present methods is a subject not having or not diagnosed as having diabetes. In some embodiments of any of the aspects, a subject treated in accordance with the present methods is a subject not administered insulin. In some embodiments of any of the aspects, the composition comprising at least one IL does not comprise insulin. In some embodiments of any of the aspects, the composition comprising at least one IL does not comprise another pharmaceutically active ingredient and/or another agent which is therapeutically effective in treating diabetes.

In some embodiments of any of the aspects, the active compound is therapeutically effective in treating obesity. In some embodiments of any of the aspects, the active compound is therapeutically effective in treating a disease associated with obesity. In some embodiments of any of the aspects, the active compound is therapeutically effective in treating a disease caused by obesity. In some embodiments of any of the aspects, the active compound is therapeutically effective in treating a disease which causes obesity. In some embodiments of any of the aspects, the active compound is therapeutically effective in treating metabolic syndrome.

In some embodiments of any of the aspects, the subject administered a composition comprising at least one IL as described herein, e.g., in combination with an active compound is a subject having, diagnosed as having, or in need of treatment for obesity, excess weight, or prevention of weight gain. In some embodiments, the subject is overweight. The methods described herein comprises methods of treating obesity, reducing weight gain, preventing weight gain, promoting weight loss, and the like. Such methods can, e.g., promote metabolic health, be pursued for aesthetic reasons, and/or prepare patients for surgical interventions which are counterindicated for those with high BMIs or weights. In some embodiments, weight loss can be medically necessary and/or medically indicated, e.g. when the subject is overweight and/or obese. In some embodiments, weight loss can be for cosmetic purposes, e.g. when the subject desires to lose weight whether or not weight loss is medically necessary and/or medically indicated.

The term “obesity” refers to excess fat in the body. Obesity can be determined by any measure accepted and utilized by those of skill in the art. Currently, an accepted measure of obesity is body mass index (BMI), which is a measure of body weight in kilograms relative to the square of height in meters. Generally, for an adult over age 20, a BMI between about 18.5 and 24.9 is considered normal, a BMI between about 25.0 and 29.9 is considered overweight, a BMI at or above about 30.0 is considered obese, and a BMI at or above about 40 is considered morbidly obese. (See, e.g., Gallagher et al. (2000) Am J Clin Nutr 72:694-701.) These BMI ranges are based on the effect of body weight on increased risk for disease. Some common conditions related to high BMI and obesity include cardiovascular disease, high blood pressure (i.e., hypertension), osteoarthritis, cancer, and diabetes. Although BMI correlates with body fat, the relation between BMI and actual body fat differs with age and gender. For example, women are more likely to have a higher percent of body fat than men for the same BMI. Furthermore, the BMI threshold that separates normal, overweight, and obese can vary, e.g. with age, gender, ethnicity, fitness, and body type, amongst other factors. In some embodiments, a subject with obesity can be a subject with a body mass index of at least about 25 kg/m² prior to administration of a treatment as described herein. In some embodiments, a subject with obesity can be a subject with a body mass index of at least about 30 kg/m² prior to administration of a treatment as described herein.

In some embodiments of any of the aspects, the subject administered a composition comprising at least one IL as described herein, e.g., in combination with at least one active compound is a subject having, diagnosed as having, or in need of treatment for a metabolic disorder or metabolic syndrome. The term “metabolic disorder” refers to any disorder associated with or aggravated by impaired or altered glucose regulation or glycemic control, such as, for example, insulin resistance. Such disorders include, but are not limited to obesity; excess adipose tissue; diabetes; fatty liver disease; non-alcoholic fatty liver disease; metabolic syndrome; dyslipidemia; hypertension; hyperglycemia; and cardiovascular disease. “Metabolic syndrome”, which is distinct from metabolic disorder, refers to a combination of medical disorders that, when occurring together, increase the risk of developing cardiovascular disease and diabetes. A number of definitions of metabolic syndrome have been established, e.g by the American Heart Association and the International Diabetes Foundation. As but one example, the WHO defines metabolic syndrome as the presence of any one of diabetes mellitus, impaired glucose tolerance, impaired fasting glucose or insulin resistance and two of the following: blood pressure equal to or greater than 140/90 mmHg, dyslipidemia, central obesity, and microalbuminuria. In some embodiments, the metabolic disorder can be selected from the group consisting of: obesity; excess adipose tissue; diabetes; and cardiovascular disease.

The uptake of many active compounds, e.g., pharmaceutically active compounds, can be improved by delivering the compounds in solvents. However, such approaches are often unsuitable for in vivo use because most such solvents demonstrate toxic side effects and/or act as irritants to the point of delivery. Described herein are methods and compositions which can provide low toxicity with improved delivery kinetics.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

A carboxylic acid is a carbonyl-bearing functional group having a formula RCOOH where R is aliphatic, heteroaliphatic, alkyl, or heteroalkyl.

As used herein, the term “alkyl” means a straight or branched, saturated aliphatic radical having a chain of carbon atoms. The term “alkyl” includes cycloalkyl or cyclic alkyl. Cₓ alkyl and Cₓ-C_{y}alkyl are typically used where X and Y indicate the number of carbon atoms in the chain. For example, C₁-C₆alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S. Examples of alkyl radicals include, but are not limited to, methyl, ethyl, propyl, isopropyl, n-butyl, tert-butyl, neopentyl, n-hexyl, and n-octyl radicals.

In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 1 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 1 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine. A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cyclyl are typically used where X and Y indicate the number of carbon atoms in the ring system. The cycloalkyl group additionally can be optionally substituted, e.g., with 1, 2, 3, or 4 substituents. Examples of cyclyl groups include, without limitation, cyclopropyl, cyclobutyl, cyclopentyl, cyclopentenyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

The terms “bicyclic” and “tricyclic” refers to fused, bridged, or joined by a single bond polycyclic ring assemblies. As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

The term “aryl” refers to monocyclic, bicyclic, or tricyclic fused aromatic ring system. Cₓ aryl and Cₓ-C_{y}aryl are typically used where X and Y indicate the number of carbon atoms in the ring system. Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. Heteroaryls include, but are not limited to, those derived from benzo[b]furan, benzo[b] thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3cjpyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

As used herein, the term “substituted” refers to independent replacement of one or more of the hydrogen atoms on the substituted moiety with substituents independently selected from, but not limited to, alkyl, alkenyl, heterocycloalkyl, alkoxy, aryloxy, hydroxy, amino, amido, alkylamino, arylamino, cyano, halo, mercapto, nitro, carbonyl, acyl, aryl and heteroaryl groups.

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls (including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of —O— alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like. The term “alkoxycarbonyl” means —C(O)O-(alkyl), such as —C(═O)OCH₃, —C(═O)OCH₂CH₃, and the like. The term “alkoxyalkyl” means -(alkyl)-O-(alkyl), such as —CH₂OCH₃, —CH₂OCH₂CH₃, and the like. The term “aryloxy” means —O-(aryl), such as —O-phenyl, —O-pyridinyl, and the like. The term “arylalkyl” means -(alkyl)-(aryl), such as benzyl (i.e., —CH₂phenyl), —CH₂-pyrindinyl, and the like. The term “arylalkyloxy” means —O-(alkyl)-(aryl), such as —O-benzyl, —O—CH₂-pyridinyl, and the like. The term “cycloalkyloxy” means —O-(cycloalkyl), such as —O-cyclohexyl, and the like. The term “cycloalkylalkyloxy” means —O-(alkyl)-(cycloalkyl, such as —OCH₂cyclohexyl, and the like. The term “aminoalkoxy” means —O-(alkyl)-NH₂, such as —OCH₂NH₂, —OCH₂CH₂NH₂, and the like. The term “mono- or di-alkylamino” means —NH(alkyl) or —N(alkyl)(alkyl), respectively, such as —NHCH₃, —N(CH₃)₂, and the like. The term “mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(CH₃)₂, and the like. The term “arylamino” means —NH(aryl), such as —NH-phenyl, —NH-pyridinyl, and the like. The term “arylalkylamino” means —NH-(alkyl)-(aryl), such as —NH-benzyl, —NHCH₂-pyridinyl, and the like. The term “alkylamino” means —NH(alkyl), such as —NHCH₃, —NHCH₂CH₃, and the like. The term “cycloalkylamino” means —NH-(cycloalkyl), such as —NH-cyclohexyl, and the like. The term “cycloalkylalkylamino” —NH-(alkyl)-(cycloalkyl), such as —NHCH₂-cyclohexyl, and the like.

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

As used here in the term “isomer” refers to compounds having the same molecular formula but differing in structure. Isomers which differ only in configuration and/or conformation are referred to as “stereoisomers.” The term “isomer” is also used to refer to an enantiomer.

The term “enantiomer” is used to describe one of a pair of molecular isomers which are mirror images of each other and non-superimposable. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate planepolarized light in different directions). Enantiomers generally have identical physical properties, such as melting points and boiling points, and also have identical spectroscopic properties. Enantiomers can differ from each other with respect to their interaction with plane-polarized light and with respect to biological activity.

The term “racemic mixture”, “racemic compound” or “racemate” refers to a mixture of the two enantiomers of one compound. An ideal racemic mixture is one wherein there is a 50:50 mixture of both enantiomers of a compound such that the optical rotation of the (+) enantiomer cancels out the optical rotation of the (−) enantiomer.

The term “resolving” or “resolution” when used in reference to a racemic mixture refers to the separation of a racemate into its two enantiomorphic forms (i.e., (+) and (−); or (R) and (S) forms). The terms can also refer to enantioselective conversion of one isomer of a racemate to a product.

The term “enantiomeric excess” or “ee” refers to a reaction product wherein one enantiomer is produced in excess of the other, and is defined for a mixture of (+)- and (−)-enantiomers, with composition given as the mole or weight or volume fraction F₍₊₎ and F₍₋₎ (where the sum of F₍₊₎ and F₍₋₎=1). The enantiomeric excess is defined as * F₍₊₎-F₍₋₎* and the percent enantiomeric excess by 100x* F₍₊₎-F₍₋₎*. The “purity” of an enantiomer is described by its ee or percent ee value (% ee).

Whether expressed as a “purified enantiomer” or a “pure enantiomer” or a “resolved enantiomer” or “a compound in enantiomeric excess”, the terms are meant to indicate that the amount of one enantiomer exceeds the amount of the other. Thus, when referring to an enantiomer preparation, both (or either) of the percent of the major enantiomer (e.g. by mole or by weight or by volume) and (or) the percent enantiomeric excess of the major enantiomer may be used to determine whether the preparation represents a purified enantiomer preparation.

The term “enantiomeric purity” or “enantiomer purity” of an isomer refers to a qualitative or quantitative measure of the purified enantiomer; typically, the measurement is expressed on the basis of ee or enantiomeric excess.

The terms “substantially purified enantiomer”, “substantially resolved enantiomer” “substantially purified enantiomer preparation” are meant to indicate a preparation (e.g. derived from non-optically active starting material, substrate, or intermediate) wherein one enantiomer has been enriched over the other, and more preferably, wherein the other enantiomer represents less than 20%, more preferably less than 10%, and more preferably less than 5%, and still more preferably, less than 2% of the enantiomer or enantiomer preparation.

The terms “purified enantiomer”, “resolved enantiomer” and “purified enantiomer preparation” are meant to indicate a preparation (e.g. derived from non-optically active starting material, substrates or intermediates) wherein one enantiomer (for example, the R-enantiomer) is enriched over the other, and more preferably, wherein the other enantiomer (for example the S-enantiomer) represents less than 30%, preferably less than 20%, more preferably less than 10% (e.g. in this particular instance, the R-enantiomer is substantially free of the S-enantiomer), and more preferably less than 5% and still more preferably, less than 2% of the preparation. A purified enantiomer may be synthesized substantially free of the other enantiomer, or a purified enantiomer may be synthesized in a stereopreferred procedure, followed by separation steps, or a purified enantiomer may be derived from a racemic mixture.

The term “enantioselectivity”, also called the enantiomeric ratio indicated by the symbol “E”, refers to the selective capacity of an enzyme to generate from a racemic substrate one enantiomer relative to the other in a product racemic mixture; in other words, it is a measure of the ability of the enzyme to distinguish between enantiomers. A nonselective reaction has an E of 1, while resolutions with E's above 20 are generally considered useful for synthesis or resolution. The enantioselectivity resides in a difference in conversion rates between the enantiomers in question. Reaction products are obtained that are enriched in one of the enantiomers; conversely, remaining substrates are enriched in the other enantiomer. For practical purposes it is generally desirable for one of the enantiomers to be obtained in large excess. This is achieved by terminating the conversion process at a certain degree of conversion.

CAGE (Choline And GEranate) is an ionic liquid comprising the cation choline (see, e.g., Structure I) and the anion geranate or geranic acid (see, e.g., Structures II and III). Preparation of CAGE can be, e.g., as described in International Patent Publication WO 2015/066647; which is incorporated by reference herein in its entirety, or as described in the examples herein.

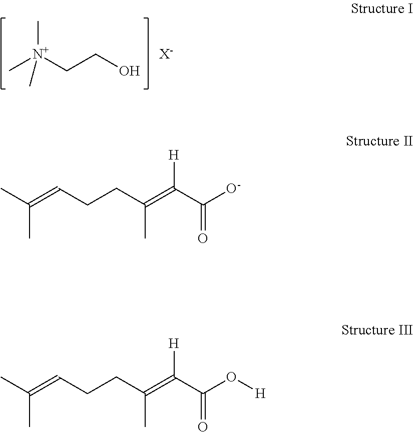

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of conditions described herein. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. the activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

In some embodiments of any of the aspects, a variant can be a polypeptide having at least 90%, at least 95%, at least 98% or greater sequence homology to one of the reference sequences provided herein and retaining the wild-type activity of that reference sequence, e.g., incretin activity. In some embodiments of any of the aspects, a variant can be a polypeptide having at least 90%, at least 95%, at least 98% or greater sequence homology to one of the naturally-occurring reference sequences provided herein and retaining the wild-type activity of that reference sequence, e.g., incretin activity. In some embodiments of any of the aspects, a variant can be a naturally-occurring polypeptide having at least 90%, at least 95%, at least 98% or greater sequence homology to one of the reference sequences provided herein and retaining the wild-type activity of that reference sequence, e.g., incretin activity.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, Jan. 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies. Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

Antibodies and/or antibody reagents can include an immunoglobulin molecule, a monoclonal antibody, a chimeric antibody, a CDR-grafted antibody, a humanized antibody, a fully human antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding portion thereof.

As used herein, the term “nanobody” or single domain antibody (sdAb) refers to an antibody comprising the small single variable domain (WH) of antibodies obtained from camelids and dromedaries. Antibody proteins obtained from members of the camel and dromedary (Camelus baclrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994; which is incorporated by reference herein in its entirety).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J. 17: 3512-3520; each of which is incorporated by reference herein in its entirety. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody. The low molecular weight and compact size further result in camelid nanobodies being extremely thermostable, stable to extreme pH and to proteolytic digestion, and poorly antigenic. See U.S. patent application 20040161738 published Aug. 19, 2004; which is incorporated by reference herein in its entirety. These features combined with the low antigenicity to humans indicate great therapeutic potential.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., cDNA. Suitable RNA can include, e.g., mRNA.

As used herein, “inhibitory nucleic acid” refers to a nucleic acid molecule which can inhibit the expression of a target, e.g., double-stranded RNAs (dsRNAs), inhibitory RNAs (iRNAs), and the like.

Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA (or modified nucleic acids as described below herein) and which mediates the targeted cleavage of an RNA transcript via an RNA-induced silencing complex (RISC) pathway. In some embodiments of any of the aspects, an iRNA as described herein effects inhibition of the expression and/or activity of a target. In some embodiments of any of the aspects, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA. In some embodiments of any of the aspects, administering an inhibitor (e.g. an iRNA) to a subject results in a decrease in the target mRNA level in the subject by at least about 5%, about 10%, about 20%, about 30%, about 40%, about 50%, about 60%, about 70%, about 80%, about 90%, about 95%, about 99%, up to and including 100% of the target mRNA level found in the subject without the presence of the iRNA.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., it can span one or more intron boundaries. The other strand (the sense strand) includes a region that is complementary to the antisense strand, such that the two strands hybridize and form a duplex structure when combined under suitable conditions. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

Exemplary embodiments of types of inhibitory nucleic acids can include, e.g,. siRNA, shRNA, miRNA, and/or amiRNA, which are well known in the art.

In some embodiments of any of the aspects, the RNA of an iRNA, e.g., a dsRNA, is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments of any of the aspects, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2-[known as a methylene (methylimino) or MMI backbone], —CH2-O—N(CH3)-CH2-, —CH2-N(CH3)-N(CH3)-CH2- and —N(CH3)-CH2-CH2-[wherein the native phosphodiester backbone is represented as —O—P—O—CH2-].

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

Modified RNAs can also contain one or more substituted sugar moieties. The iRNAs, e.g., dsRNAs, described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO] mCH3, O(CH2).nOCH3, O(CH2)nNH2, O(CH2) nCH3, O(CH2)nONH2, and O(CH₂)nON[(CH2)nCH3)]2, where n and m are from 1 to about 10. In some embodiments of any of the aspects, dsRNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF₃, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments of any of the aspects, the modification includes a 2′ methoxyethoxy (2′-O—CH2CH2OCH3, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar.

An inhibitory nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

The preparation of the modified nucleic acids, backbones, and nucleobases described above are well known in the art.

Another modification of an inhibitory nucleic acid featured in the invention involves chemically linking to the inhibitory nucleic acid to one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a recombinant vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a condition or disease described herein. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “effective amount” means an amount of a composition sufficient to provide at least some amelioration of the symptoms associated with the condition. In one embodiment, the “effective amount” means an amount of a composition would decrease the markers or symptoms of the condition in a subject having the condition.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” or “comprises” is used in reference to methods and compositions, and respective component(s) thereof, that are essential to the invention, yet open to the inclusion of unspecified elements, whether essential or not. As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of administering at least one active compound, the method comprising administering the active compound in combination with at least one ionic liquid comprising:
   a hydrophobic anion comprising a carboxylic acid having a pKa of at least 4.0 and a Log P of at least 1.0; and
   a cation comprising a quaternary ammonium.
2. A method of reducing weight/weight gain or treating obesity, diabetes, ulcers, cancer, or fibrosis in a subject in need thereof, the method comprising administering a composition comprising at least one ionic liquid comprising:
   a hydrophobic anion comprising a carboxylic acid having a pKa of at least 4.0 and a Log P of at least 1.0; and
   a cation comprising a quaternary ammonium to the subject.
3. The method of paragraph 2, wherein the composition does not comprise a therapeutic agent other than the at least one ionic liquid.
4. The method of paragraph 2, wherein the composition further comprises an active compound other than the at least one ionic liquid.
5. The method of any of the preceding paragraphs, wherein the anion has a pKa of at least 4.5.
6. The method of any of the preceding paragraphs, wherein the anion has a pKa of at least 5.0.
7. The method of any of the preceding paragraphs, wherein the anion has a Log P of at least 2.0.
8. The method of any of the preceding paragraphs, wherein the anion has a Log P of at least 2.5.
9. The method of any of the preceding paragraphs, wherein the anion has a Log P of at least 2.75.
10. The method of any of the preceding paragraphs, wherein the anion comprises a carbon chain of at least 8 carbons.
11. The method of any of the preceding paragraphs, wherein the anion is an alkene.
12. The method of any of the preceding paragraphs, wherein the anion is geranic acid, octanoic acid, or citronellic acid.
13. The method of any of the preceding paragraphs, wherein the cation has a molar mass equal to or greater than choline.
14. The method of any of the preceding paragraphs, wherein the quarternary ammonium has the structure of NR₄⁺ and at least one R group comprises a hydroxy group.
15. The method of any of the preceding paragraphs, wherein the quarternary ammonium has the structure of NR₄⁺ and only one R group comprises a hydroxy group.
16. The method of any of the preceding paragraphs, wherein the cation is C1, C6, or C7.
17. The method of any of the preceding paragraphs, wherein the cation is selected from choline, C1, C6, and C7 and the anion is citronellic acid.
18. The method of any of the preceding paragraphs, wherein the cation is C1 and the anion is citronellic acid.
19. The method of any of the preceding paragraphs, wherein the cation is selected from C1, C6, and C7 and the anion is geranic acid.
20. The method of any of the preceding paragraphs, wherein the ionic liquid is choline: citronellic acid, C1: geranic acid, or C1: citronellic acid.
21. The method of any of the preceding paragraphs, wherein the ionic liquid is not CAGE.
22. The method of any of the preceding paragraphs, wherein the ionic liquid has less than 20 cross peaks as measured by Nuclear Overhauser Effect SpectroscopY (NOESY).
23. The method of any of the preceding paragraphs, wherein the ionic liquid has less than 10 cross peaks as measured by Nuclear Overhauser Effect SpectroscopY (NOESY).
24. The method of any of the preceding paragraphs, wherein the ionic liquid has less than 5 cross peaks as measured by Nuclear Overhauser Effect SpectroscopY (NOESY).
25. The method of any of the preceding paragraphs, wherein the administration is transdermal.
26. The method of any of the preceding paragraphs, wherein the administration is transdermal, to a mucus membrane, oral, subcutaneous, intradermal, parenteral, intratumoral, or intravenous.
27. The method of paragraph 26, wherein the mucus membrane is nasal, oral, or vaginal.
28. The method of any of the preceding paragraphs, wherein the ionic liquid is at a concentration of at least 0.1% w/v.
29. The method of any of the preceding paragraphs, wherein the ionic liquid is at a concentration of from about 10 to about 70% w/v.
30. The method of any of the preceding paragraphs, wherein the ionic liquid is at a concentration of from about 30 to about 50% w/v.
31. The method of any of the preceding paragraphs, wherein the ionic liquid is at a concentration of from about 30 to about 40% w/v.
32. The method of any of the preceding paragraphs, wherein the ionic liquid comprises a ratio of cation to anion of from about 2:1 to about 1:10.
33. The method of any of the preceding paragraphs, wherein the ionic liquid comprises a ratio of cation to anion of from about 1:1 to about 1:4.
34. The method of any of the preceding paragraphs, wherein the ionic liquid comprises a ratio of cation to anion of about 1:2.
35. The method of any of the preceding paragraphs, wherein the ionic liquid has a cation:anion ratio of less than 1:1.
36. The method of any of the preceding paragraphs, wherein the active compound is hydrophobic.
37. The method of any of the preceding paragraphs, wherein the active compound is hydrophilic.
38. The method of any of the preceding paragraphs, wherein the active compound comprises a polypeptide.
39. The method of any of the preceding paragraphs, wherein the active compound has a molecular weight of greater than 450.
40. The method of any of the preceding paragraphs, wherein the active compound has a molecular weight of greater than 500.
41. The method of any of the preceding paragraphs, wherein the active compound comprises an antibody or antibody reagent.
42. The method of any of the preceding paragraphs, wherein the active compound comprises insulin, acarbose, ruxolitinib, or a GLP-1 polypeptide or mimetic or analog thereof
43. The method of any of the preceding paragraphs, wherein the combination and/or composition is administered once.
44. The method of any of the preceding paragraphs, wherein the combination and/or composition is administered in multiple doses.
45. The method of any of the preceding paragraphs, wherein the active compound and/or composition is provided at a dosage of 1-20 mg/kg.
46. The method of any of the preceding paragraphs, wherein the active compound and the ionic liquid are further in combination with at least one non-ionic surfactant.
47. The method of any of the preceding paragraphs, wherein the combination and/or composition further comprises a further pharmaceutically acceptable carrier.
48. The method of any of the preceding paragraphs, wherein the administration is oral and the combination and/or composition is provided in a degradable capsule.
49. The method of any of the preceding paragraphs, wherein the combination is an admixture.
50. The method of any of the preceding paragraphs, wherein the combination and/or composition is provided in one or more nanoparticles.
51. The method of any of the preceding paragraphs, wherein the combination is provided in the form of one or more nanoparticles comprising the active compound, the nanoparticles in solution or suspension in a composition comprising the ionic liquid.
52. A composition comprising at least one ionic liquid comprising:
   a hydrophobic anion comprising a carboxylic acid having a pKa of at least 4.0 and a Log P of at least 1.0; and
   a cation comprising a quaternary ammonium.
53. The composition of paragraph 52, wherein the anion has a pKa of at least 4.5.
54. The composition of any of paragraphs 52-53, wherein the anion has a pKa of at least 5.0.
55. The composition of any of paragraphs 52-54, wherein the anion has a Log P of at least 2.0.
56. The composition of any of paragraphs 52-55, wherein the anion has a Log P of at least 2.5.
57. The composition of any of paragraphs 52-56, wherein the anion has a Log P of at least 2.75.
58. The composition of any of paragraphs 52-57, wherein the anion comprises a carbon chain of at least 8 carbons.
59. The composition of any of paragraphs 52-58, wherein the anion is an alkene.
60. The composition of any of paragraphs 52-59, wherein the anion is geranic acid, octanoic acid, or citronellic acid.
61. The composition of any of paragraphs 52-60, wherein the cation has a molar mass equal to or greater than choline.
62. The composition of any of paragraphs 52-61, wherein the quarternary ammonium has the structure of NR₄⁺ and at least one R group comprises a hydroxy group.
63. The composition of any of paragraphs 52-62, wherein the quarternary ammonium has the structure of NR₄⁺ and only one R group comprises a hydroxy group.
64. The composition of any of paragraphs 52-63, wherein the cation is C1, C6, or C7.
65. The composition of any of paragraphs 52-64, wherein the cation is selected from choline, C1, C6, and C7 and the anion is citronellic acid.
66. The composition of any of paragraphs 52-65, wherein the cation is C1 and the anion is citronellic acid.
67. The composition of any of paragraphs 52-66, wherein the cation is selected from C1, C6, and C7 and the anion is geranic acid.
68. The composition of any of paragraphs 52-67, wherein the ionic liquid is choline: citronellic acid, C1: geranic acid, or C1: citronellic acid.
69. The composition of any of paragraphs 52-68, wherein the ionic liquid is not CAGE.
70. The composition of any of paragraphs 52-69, wherein the ionic liquid comprises a ratio of cation to anion of from about 2:1 to about 1:10.
71. The composition of any of paragraphs 52-70, wherein the ionic liquid comprises a ratio of cation to anion of from about 1:1 to about 1:4.
72. The composition of any of paragraphs 52-71, wherein the ionic liquid comprises a ratio of cation to anion of about 1:2.
73. The composition of any of paragraphs 52-72, wherein the ionic liquid has a cation:anion ratio of less than 1:1.
74. The composition of any of paragraphs 52-73, wherein the ionic liquid has a cation:anion ratio with an excess of anion.
75. The composition of any of paragraphs 52-74, wherein the ionic liquid has less than 20 cross peaks as measured by Nuclear Overhauser Effect SpectroscopY (NOESY).
76. The composition of any of paragraphs 52-75, wherein the ionic liquid has less than 10 cross peaks as measured by Nuclear Overhauser Effect SpectroscopY (NOESY).
77. The composition of any of paragraphs 52-76, wherein the ionic liquid has less than 5 cross peaks as measured by Nuclear Overhauser Effect SpectroscopY (NOESY).
78. The composition of any of paragraphs 52-77, further comprising at least one active compound in combination with the at least one ionic liquid.
79. The composition of paragraph 78, wherein the active compound is hydrophobic.
80. The composition of any of paragraphs 78-79, wherein the active compound is hydrophilic.
81. The composition of any of paragraphs 78-80, wherein the active compound comprises a polypeptide.
82. The composition of any of paragraphs 78-81, wherein the active compound has a molecular weight of greater than 450.
83. The composition of any of paragraphs 78-82, wherein the active compound has a molecular weight of greater than 500.
84. The composition of any of paragraphs 78-83, wherein the active compound comprises an antibody or antibody reagent.
85. The composition of any of paragraphs 78-84, wherein the active compound comprises insulin, acarbose, ruxolitinib, or a GLP-1 polypeptide or mimetic or analog thereof.
86. The composition of any of paragraphs 52-85, wherein the ionic liquid is at a concentration of at least 0.1% w/v.
87. The composition of any of paragraphs 52-86, wherein the ionic liquid is at a concentration of from about 10 to about 70% w/v.
88. The composition of any of paragraphs 52-87, wherein the ionic liquid is at a concentration of from about 30 to about 50% w/v.
89. The composition of any of paragraphs 52-88, wherein the ionic liquid is at a concentration of from about 30 to about 40% w/v.
90. The composition of any of paragraphs 52-89, wherein the compostition is formulated for transdermal administration.
91. The composition of any of paragraphs 52-90, wherein the composition is formulated for administration transdermally, to a mucus membrane, orally, subcutaneously, intradermally, parenterally, intratumorally, or intravenously.
92. The composition of paragraph 91, wherein the mucus membrane is nasal, oral, or vaginal.
93. The composition of any of paragraphs 78-92, wherein the active compound is provided at a dosage of 1-20 mg/kg.
94. The composition of any of paragraphs 52-93, further comprising at least one non-ionic surfactant.
95. The composition of any of paragraphs 52-94, further comprising a pharmaceutically acceptable carrier.
96. The composition of any of paragraphs 52-95, wherein the composition is provided in a degradable capsule.
97. The composition of any of paragraphs 78-96, wherein the composition is an admixture.
98. The composition of any of paragraphs 52-97, wherein the composition is provided in one or more nanoparticles.
99. The composition of any of paragraphs 52-98, comprising one or more nanoparticles comprising the active compound, the nanoparticles in solution or suspension in a composition comprising the ionic liquid.

Some embodiments of the technology described herein can be defined according to any of the following numbered paragraphs:
1. A method of administering at least one active compound, the method comprising administering the active compound in combination with at least one ionic liquid comprising:
   a hydrophobic anion comprising a carboxylic acid having a pKa of at least 4.0 and a Log P of at least 1.0; and
   a cation comprising a quaternary ammonium.
2. A method of reducing weight/weight gain or treating obesity, diabetes, ulcers, cancer, or fibrosis in a subject in need thereof, the method comprising administering a composition comprising at least one ionic liquid comprising:
   a hydrophobic anion comprising a carboxylic acid having a pKa of at least 4.0 and a Log P of at least 1.0; and
   a cation comprising a quaternary ammonium to the subject.
3. The method of paragraph 2, wherein the composition does not comprise a therapeutic agent other than the at least one ionic liquid.
4. The method of paragraph 2, wherein the composition further comprises an active compound other than the at least one ionic liquid.
5. The method of any of the preceding paragraphs, wherein the anion has a pKa of at least 4.5.
6. The method of any of the preceding paragraphs, wherein the anion has a pKa of at least 5.0.
7. The method of any of the preceding paragraphs, wherein the anion has a pKa of at least 4.895.
8. The method of any of the preceding paragraphs, wherein the anion has a pKa of 4.5-5.5.
9. The method of any of the preceding paragraphs, wherein the anion has a pKa of 4.895-5.19.
10. The method of any of the preceding paragraphs, wherein the anion has a Log P of at least 2.0.
11. The method of any of the preceding paragraphs, wherein the anion has a Log P of at least 2.5.
12. The method of any of the preceding paragraphs, wherein the anion has a Log P of at least 2.75.
13. The method of any of the preceding paragraphs, wherein the anion has a Log P of at least 2.8.
14. The method of any of the preceding paragraphs, wherein the anion has a Log P of 2.5-3.5.
15. The method of any of the preceding paragraphs, wherein the anion has a Log P of 2.8-3.01.
16. The method of any of the preceding paragraphs, wherein the anion comprises a carbon chain of at least 8 carbons.
17. The method of any of the preceding paragraphs, wherein the anion comprises a carbon chain with an 8 carbon backbone.
18. The method of any of the preceding paragraphs, wherein the anion is geranic acid, octenoic acid, octanoic acid, or citronellic acid.
19. The method of any of the preceding paragraphs, wherein the anion is octenoic acid, octanoic acid, or citronellic acid.
20. The method of any of the preceding paragraphs, wherein the anion is an alkene.
21. The method of any of the preceding paragraphs, wherein the anion is geranic acid, octanoic acid, or citronellic acid.
22. The method of any of the preceding paragraphs, wherein the cation has a molar mass equal to or greater than choline.
23. The method of any of the preceding paragraphs, wherein the quarternary ammonium has the structure of NR₄⁺ and at least one R group comprises a hydroxy group.
24. The method of any of the preceding paragraphs, wherein the quarternary ammonium has the structure of NR₄⁺ and only one R group comprises a hydroxy group.
25. The method of any of the preceding paragraphs, wherein the cation is C1, C6, or C7.
26. The method of any of the preceding paragraphs, wherein the cation is selected from choline, C1, C6, and C7 and the anion is citronellic acid.
27. The method of any of the preceding paragraphs, wherein the cation is C1 and the anion is citronellic acid.
28. The method of any of the preceding paragraphs, wherein the cation is selected from C1, C6, and C7 and the anion is geranic acid.
29. The method of any of the preceding paragraphs, wherein the ionic liquid is choline: citronellic acid, C1: geranic acid, or C1: citronellic acid.
30. The method of any of paragraphs 1-16, wherein the cation is selected from choline, C1, C6, and C7 and the anion is selected from citronellic acid, octanoic acid, and octenoic acid.
31. The method of any of paragraphs 1-16, wherein the cation is choline and the anion is selected from citronellic acid, octanoic acid, and octenoic acid.
32. The method of any of paragraphs 1-16, wherein the ionic liquid is choline: citronellic acid, choline: octanoic acid, or choline: octenoic acid.
33. The method of any of the preceding paragraphs, wherein the ionic liquid is not CAGE.
34. The method of any of the preceding paragraphs, wherein the ionic liquid has less than 20 cross peaks as measured by Nuclear Overhauser Effect SpectroscopY (NOESY).
35. The method of any of the preceding paragraphs, wherein the ionic liquid has less than 10 cross peaks as measured by Nuclear Overhauser Effect SpectroscopY (NOESY).
36. The method of any of the preceding paragraphs, wherein the ionic liquid has less than 5 cross peaks as measured by Nuclear Overhauser Effect SpectroscopY (NOESY).
37. The method of any of the preceding paragraphs, wherein the administration is transdermal.
38. The method of any of the preceding paragraphs, wherein the administration is transdermal, to a mucus membrane, oral, subcutaneous, intradermal, parenteral, intratumoral, or intravenous.
39. The method of paragraph 26, wherein the mucus membrane is nasal, oral, or vaginal.
40. The method of any of the preceding paragraphs, wherein the administration is oral.
41. The method of any of the preceding paragraphs, wherein the ionic liquid is at a concentration of at least 0.1% w/v.
42. The method of any of the preceding paragraphs, wherein the ionic liquid is at a concentration of from about 10 to about 70% w/v.
43. The method of any of the preceding paragraphs, wherein the ionic liquid is at a concentration of from about 30 to about 50% w/v.
44. The method of any of the preceding paragraphs, wherein the ionic liquid is at a concentration of from about 30 to about 40% w/v.
45. The method of any of the preceding paragraphs, wherein the ionic liquid comprises a ratio of cation to anion of from about 2:1 to about 1:10.
46. The method of any of the preceding paragraphs, wherein the ionic liquid comprises a ratio of cation to anion of from about 1:1 to about 1:4.
47. The method of any of the preceding paragraphs, wherein the ionic liquid comprises a ratio of cation to anion of about 1:2.
48. The method of any of the preceding paragraphs, wherein the ionic liquid has a cation:anion ratio of less than 1:1.
49. The method of any of the preceding paragraphs, wherein the active compound is hydrophobic.
50. The method of any of the preceding paragraphs, wherein the active compound is hydrophilic.
51. The method of any of the preceding paragraphs, wherein the active compound comprises a polypeptide.
52. The method of any of the preceding paragraphs, wherein the active compound has a molecular weight of greater than 450.
53. The method of any of the preceding paragraphs, wherein the active compound has a molecular weight of greater than 500.
54. The method of any of the preceding paragraphs, wherein the active compound comprises an antibody or antibody reagent.
55. The method of any of the preceding paragraphs, wherein the active compound comprises insulin, acarbose, ruxolitinib, or a GLP-1 polypeptide or mimetic or analog thereof
56. The method of any of the preceding paragraphs, wherein the combination and/or composition is administered once.
57. The method of any of the preceding paragraphs, wherein the combination and/or composition is administered in multiple doses.
58. The method of any of the preceding paragraphs, wherein the active compound and/or composition is provided at a dosage of 1-20 mg/kg.
59. The method of any of the preceding paragraphs, wherein the active compound and the ionic liquid are further in combination with at least one non-ionic surfactant.
60. The method of any of the preceding paragraphs, wherein the combination and/or composition further comprises a further pharmaceutically acceptable carrier.
61. The method of any of the preceding paragraphs, wherein the administration is oral and the combination and/or composition is provided in a degradable capsule.
62. The method of any of the preceding paragraphs, wherein the combination is an admixture.
63. The method of any of the preceding paragraphs, wherein the combination and/or composition is provided in one or more nanoparticles.
64. The method of any of the preceding paragraphs, wherein the combination is provided in the form of one or more nanoparticles comprising the active compound, the nanoparticles in solution or suspension in a composition comprising the ionic liquid.
65. A composition comprising at least one ionic liquid comprising:
   a hydrophobic anion comprising a carboxylic acid having a pKa of at least 4.0 and a Log P of at least 1.0; and
   a cation comprising a quaternary ammonium.
66. The composition of paragraph 65, wherein the anion has a pKa of at least 4.5.
67. The composition of paragraph 65, wherein the anion has a pKa of at least 4.895.
68. The composition of paragraph 65, wherein the anion has a pKa of 4.5-5.5.
69. The composition of paragraph 65, wherein the anion has a pKa of 4.895-5.19.
70. The composition of any of paragraphs 65-69, wherein the anion has a pKa of at least 5.0.
71. The composition of any of paragraphs 65-69, wherein the anion has a Log P of at least 2.0.
72. The composition of any of paragraphs 65-69, wherein the anion has a Log P of at least 2.5.
73. The composition of any of paragraphs 65-69, wherein the anion has a Log P of at least 2.75.
74. The composition of any of paragraphs 65-69, wherein the anion has a Log P of at least 2.8.
75. The composition of any of paragraphs 65-69, wherein the anion has a Log P of 2.5-3.5.
76. The composition of any of paragraphs 65-69, wherein the anion has a Log P of 2.8-3.01.
77. The composition of any of paragraphs 65-76, wherein the anion comprises a carbon chain of at least 8 carbons.
78. The composition of any of paragraphs 65-76, wherein the anion comprises a carbon chain with an 8 carbon backbone.
79. The composition of any of paragraphs 65-76, wherein the anion is geranic acid, octenoic acid, octanoic acid, or citronellic acid.
80. The composition of any of paragraphs 65-76, wherein the anion is octenoic acid, octanoic acid, or citronellic acid.
81. The composition of any of paragraphs 65-80, wherein the anion is an alkene.
82. The composition of any of paragraphs 65-81, wherein the anion is geranic acid, octanoic acid, or citronellic acid.
83. The composition of any of paragraphs 65-82, wherein the cation has a molar mass equal to or greater than choline.
84. The composition of any of paragraphs 65-83, wherein the quarternary ammonium has the structure of NR₄⁺ and at least one R group comprises a hydroxy group.
85. The composition of any of paragraphs 65-84, wherein the quarternary ammonium has the structure of NR₄⁺ and only one R group comprises a hydroxy group.
86. The composition of any of paragraphs 65-85, wherein the cation is C1, C6, or C7.
87. The composition of any of paragraphs 65-86, wherein the cation is selected from choline, C1, C6, and C7 and the anion is citronellic acid.
88. The composition of any of paragraphs 65-87, wherein the cation is C1 and the anion is citronellic acid.
89. The composition of any of paragraphs 65-88, wherein the cation is selected from C1, C6, and C7 and the anion is geranic acid.
90. The composition of any of paragraphs 65-89, wherein the ionic liquid is choline: citronellic acid, C1: geranic acid, or C1: citronellic acid.
91. The composition of any of paragraphs 65-90, wherein the cation is selected from choline, C1, C6, and C7 and the anion is selected from citronellic acid, octanoic acid, and octenoic acid.
92. The composition of any of paragraphs 65-91, wherein the cation is choline and the anion is selected from citronellic acid, octanoic acid, and octenoic acid.
93. The composition of any of paragraphs 65-92, wherein the ionic liquid is choline: citronellic acid, choline: octanoic acid, or choline: octenoic acid.
94. The composition of any of paragraphs 65-93, wherein the ionic liquid is not CAGE.
95. The composition of any of paragraphs 65-94, wherein the ionic liquid comprises a ratio of cation to anion of from about 2:1 to about 1:10.
96. The composition of any of paragraphs 65-95, wherein the ionic liquid comprises a ratio of cation to anion of from about 1:1 to about 1:4.
97. The composition of any of paragraphs 65-96, wherein the ionic liquid comprises a ratio of cation to anion of about 1:2.
98. The composition of any of paragraphs 65-97, wherein the ionic liquid has a cation:anion ratio of less than 1:1.
99. The composition of any of paragraphs 65-98, wherein the ionic liquid has a cation:anion ratio with an excess of anion.
100. The composition of any of paragraphs 65-99, wherein the ionic liquid has less than 20 cross peaks as measured by Nuclear Overhauser Effect SpectroscopY (NOESY).
101. The composition of any of paragraphs 65-100, wherein the ionic liquid has less than 10 cross peaks as measured by Nuclear Overhauser Effect SpectroscopY (NOESY).
102. The composition of any of paragraphs 65-101, wherein the ionic liquid has less than 5 cross peaks as measured by Nuclear Overhauser Effect SpectroscopY (NOESY).
103. The composition of any of paragraphs 65-102, further comprising at least one active compound in combination with the at least one ionic liquid.
104. The composition of paragraph 103, wherein the active compound is hydrophobic.
105. The composition of any of paragraphs 103-104, wherein the active compound is hydrophilic.
106. The composition of any of paragraphs 103-105, wherein the active compound comprises a polypeptide.
107. The composition of any of paragraphs 103-106, wherein the active compound has a molecular weight of greater than 450.
108. The composition of any of paragraphs 103-107, wherein the active compound has a molecular weight of greater than 500.
109. The composition of any of paragraphs 103-108, wherein the active compound comprises an antibody or antibody reagent.
110. The composition of any of paragraphs 103-109, wherein the active compound comprises insulin, acarbose, ruxolitinib, or a GLP-1 polypeptide or mimetic or analog thereof
111. The composition of any of paragraphs 65-110, wherein the ionic liquid is at a concentration of at least 0.1% w/v.
112. The composition of any of paragraphs 65-111, wherein the ionic liquid is at a concentration of from about 10 to about 70% w/v.
113. The composition of any of paragraphs 65-112, wherein the ionic liquid is at a concentration of from about 30 to about 50% w/v.
114. The composition of any of paragraphs 65-113, wherein the ionic liquid is at a concentration of from about 30 to about 40% w/v.
115. The composition of any of paragraphs 65-114, wherein the composition is formulated for transdermal administration.
116. The composition of any of paragraphs 65-115, wherein the composition is formulated for administration transdermally, to a mucus membrane, orally, subcutaneously, intradermally, parenterally, intratumorally, or intravenously.
117. The composition of paragraph 116, wherein the mucus membrane is nasal, oral, or vaginal.
118. The composition of any of paragraphs 65-117, wherein the composition is formulated for oral administration.
119. The composition of any of paragraphs 103-118, wherein the active compound is provided at a dosage of 1-20 mg/kg.
120. The composition of any of paragraphs 65-119, further comprising at least one non-ionic surfactant.
121. The composition of any of paragraphs 65-120, further comprising a pharmaceutically acceptable carrier.
122. The composition of any of paragraphs 65-121, wherein the composition is provided in a degradable capsule.
123. The composition of any of paragraphs 65-122, wherein the composition is an admixture.
124. The composition of any of paragraphs 65-123, wherein the composition is provided in one or more nanoparticles.
125. The composition of any of paragraphs 65-124, comprising one or more nanoparticles comprising the active compound, the nanoparticles in solution or suspension in a composition comprising the ionic liquid.
126. A method of designing and/or identifying an ionic liquid comprising two ions, wherein one ion is a cation and the other ion is an anion, the method comprising:
   a. selecting one of the two ions of the ionic liquid; and
   b. selecting the other ion to minimize inter-ionic interactions.
127. A method of designing and/or identifying an ionic liquid comprising two ions, wherein one ion is a cation and the other ion is an anion, the method comprising:
   a. selecting the cation; and
   b. selecting the anion to minimize inter-ionic interactions.
128. A method of designing and/or identifying an ionic liquid comprising two ions, wherein one ion is a cation and the other ion is an anion, the method comprising:
   a. selecting the anion; and
   b. selecting the cation to minimize inter-ionic interactions.
129. A method of designing and/or identifying an ionic liquid comprising two ions, wherein one ion is a cation and the other ion is an anion, from a pool of candidate cations and a pool of candidate anions, the method comprising:
   a. selecting one of the two ions of the ionic liquid from the pool of candidate ions; and
   b. selecting from the other pool of candidate ions the ion which most minimizes inter-ionic interactions with the ion selected in step a.
130. A method of designing and/or identifying an ionic liquid comprising two ions, wherein one ion is a cation and the other ion is an anion, from a pool of candidate cations and a pool of candidate anions, the method comprising:
   a. selecting the cation from the pool of candidate cations;
   b. selecting from the pool of candidate anions the anion which most minimizes inter-ionic interactions with the cation selected in step a.
131. A method of designing and/or identifying an ionic liquid comprising two ions, wherein one ion is a cation and the other ion is an anion, from a pool of candidate cations and a pool of candidate anions, the method comprising:
   a. selecting the cation from the pool of candidate anions;
   b. selecting from the pool of candidate cations the anion which most minimizes inter-ionic interactions with the anion selected in step a.
132. The method of any of paragraphs 126-131, wherein the ionic liquid is selected or designed for transdermal administration.
133. The method of any of paragraphs 126-132, wherein the ionic liquid is selected or designed for administration transdermally, to a mucus membrane, orally, subcutaneously, intradermally, parenterally, intratumorally, or intravenously.
134. The method of paragraph 133, wherein the mucus membrane is nasal, oral, or vaginal.
135. The method of any of paragraphs 126-134, wherein the ionic liquid is selected or designed for oral administration.
136. The method of any of paragraphs 126-135, wherein the ionic liquid is selected or designed for delivery of an active compound.
137. The method of any of paragraphs 126-136, wherein the cation comprises, or selecting the cation comprises selection a cation that comprises a quaternary ammonium; and the anion comprises or selecting an anion comprises selecting a hydrophobic anion comprising a carboxylic acid having a pKa of at least 4.0 and a Log P of at least 1.0.
138. The method of any of paragraphs 126-137, wherein the anion has, or selecting an anion comprises selecting an anion that has, a pKa of at least 4.5.
139. The method of any of paragraphs 126-138, wherein the anion has, or selecting an anion comprises selecting an anion that has, a pKa of at least 4.895.
140. The method of any of paragraphs 126-138, wherein the anion has, or selecting an anion comprises selecting an anion that has, a pKa of 4.5-5.5.
141. The method of any of paragraphs 126-140, wherein the anion has, or selecting an anion comprises selecting an anion that has, a pKa of 4.895-5.19.
142. The method of any of paragraphs 126-141, wherein the anion has, or selecting an anion comprises selecting an anion that has, a pKa of at least 5.0.
143. The method of any of paragraphs 126-142, wherein the anion has, or selecting an anion comprises selecting an anion that has, a Log P of at least 2.0.
144. The method of any of paragraphs 126-143, wherein the anion has, or selecting an anion comprises selecting an anion that has, a Log P of at least 2.5.
145. The method of any of paragraphs 126-144, wherein the anion has, or selecting an anion comprises selecting an anion that has, a Log P of at least 2.75.
146. The method of any of paragraphs 126-145, wherein the anion has, or selecting an anion comprises selecting an anion that has, a Log P of at least 2.8.
147. The method of any of paragraphs 126-146, wherein the anion has, or selecting an anion comprises selecting an anion that has, a Log P of 2.5-3.5.
148. The method of any of paragraphs 126-147, wherein the anion has, or selecting an anion comprises selecting an anion that has, a Log P of 2.8-3.01.
149. The method of any of paragraphs 126-148, wherein the anion comprises, or selecting an anion comprises selecting an anion that comprises, a carbon chain of at least 8 carbons.
150. The method of any of paragraphs 126-149, wherein the anion comprises, or selecting an anion comprises selecting an anion that comprises, a carbon chain with an 8 carbon backbone.
151. The method of any of paragraphs 126-150, wherein the anion is, or selecting an anion comprises selecting an anion that is, geranic acid, octenoic acid, octanoic acid, or citronellic acid.
152. The method of any of paragraphs 126-151, wherein the anion is, or selecting an anion comprises selecting an anion that is, octenoic acid, octanoic acid, or citronellic acid.
153. The method of any of paragraphs 126-152, wherein the anion is, or selecting an anion comprises selecting an anion that is, an alkene.
154. The method of any of paragraphs 126-153, wherein the anion is, or selecting an anion comprises selecting an anion that is, geranic acid, octanoic acid, or citronellic acid.
155. The method of any of paragraphs 126-154, wherein the cation has, or selecting a cation comprises selecting a cation that has, a molar mass equal to or greater than choline.
156. The method of any of paragraphs 126-155, wherein the quarternary ammonium has the structure of NR₄+ and at least one R group comprises a hydroxy group.
157. The method of any of paragraphs 126-156, wherein the quarternary ammonium has the structure of NR₄+ and only one R group comprises a hydroxy group.
158. The method of any of paragraphs 126-157, wherein the cation is, or selecting a cation comprises selecting a cation that is, C1, C6, or C7.
159. The method of any of paragraphs 126-158, wherein the cation is, or selecting a cation comprises selecting a cation that is, selected from choline, C1, C6, and C7 and the anion is citronellic acid.
160. The method of any of paragraphs 126-159, wherein the cation is, or selecting a cation comprises selecting a cation that is, selected from C1, C6, and C7 and the anion is geranic acid.
161. The method of any of paragraphs 126-160, wherein the cation is, or selecting a cation comprises selecting a cation that is, choline, C1, C6, or C7 and selecting the anion comprises selecting an anion that is citronellic acid, octanoic acid, or octenoic acid.
162. The method of any of paragraphs 126-161, wherein the cation is choline and selecting the anion comprises selecting an anion that is citronellic acid, octanoic acid, or octenoic acid.
163. The method of any of paragraphs 126-162, wherein the ionic liquid is not CAGE.
164. The method of any of paragraphs 126-163, wherein the ionic liquid comprises a ratio of cation to anion of from about 2:1 to about 1:10.
165. The method of any of paragraphs 126-164, wherein the ionic liquid comprises a ratio of cation to anion of from about 1:1 to about 1:4.
166. The method of any of paragraphs 126-165, wherein the ionic liquid comprises a ratio of cation to anion of about 1:2.
167. The method of any of paragraphs 126-166, wherein the ionic liquid has a cation:anion ratio of less than 1:1.
168. The method of any of paragraphs 126-167, wherein the ionic liquid has a cation:anion ratio with an excess of anion.
169. The method of any of paragraphs 126-168, wherein minimizing inter-ionic interaction comprises minimizing the number of cross peaks as measured by Nuclear Overhauser Effect SpectroscopY (NOESY).
170. The method of any of paragraphs 126-169, wherein a cation and anion minimize inter-ionic interaction if they have less than 20 cross peaks as measured by Nuclear Overhauser Effect SpectroscopY (NOESY).
171. The method of any of paragraphs 126-170, wherein a cation and anion minimize inter-ionic interaction if they have less than 10 cross peaks as measured by Nuclear Overhauser Effect SpectroscopY (NOESY).
172. The method of any of paragraphs 126-171, wherein a cation and anion minimize inter-ionic interaction if they have less than 5 cross peaks as measured by Nuclear Overhauser Effect SpectroscopY (NOESY).
173. The method of any of paragraphs 126-172, wherein the active compound is hydrophobic.
174. The method of any of paragraphs 126-173, wherein the active compound is hydrophilic.
175. The method of any of paragraphs 126-174, wherein the active compound comprises a polypeptide.
176. The method of any of paragraphs 126-175, wherein the active compound has a molecular weight of greater than 450.
177. The method of any of paragraphs 126-176, wherein the active compound has a molecular weight of greater than 500.
178. The method of any of paragraphs 126-177, wherein the active compound comprises an antibody or antibody reagent.
179. The method of any of paragraphs 126-178, wherein the active compound comprises insulin, acarbose, ruxolitinib, or a GLP-1 polypeptide or mimetic or analog thereof
180. The method of any of paragraphs 126-179, wherein the ionic liquid is at a concentration of at least 0.1% w/v.
181. The method of any of paragraphs 126-180, wherein the ionic liquid is at a concentration of from about 10 to about 70% w/v.
182. The method of any of paragraphs 126-181, wherein the ionic liquid is at a concentration of from about 30 to about 50% w/v.
183. The method of any of paragraphs 126-182, wherein the ionic liquid is at a concentration of from about 30 to about 40% w/v.
184. The method of any of paragraphs 126-183, wherein the active compound is provided at a dosage of 1-20 mg/kg.
185. The method of any of paragraphs 126-184, wherein the ionic liquid is designed or selected to be provided in a degradable capsule.
186. The method of any of paragraphs 126-185, wherein the ionic liquid and active compound are in admixture.
187. The method of any of paragraphs 126-186, wherein the ionic liquid and optionally the active compound are selected or designed to be provided in one or more nanoparticles.
188. The method of any of paragraphs 126-187, wherein the ionic liquid is in solution or suspension in a composition with one or more nanoparticles comprising the active compound.

### EXAMPLES

### Example 1

Choline-based ionic liquids, in particular choline and geranic acid (CAGE), have been used to enhance the delivery of several small and large molecules across the skin. However, detailed studies outlining the design principles of ILs for transdermal drug delivery are still lacking. Using two model drugs of differing hydrophilicities, acarbose and ruxolitinib, and 16 ionic liquids of varying cations and anions, we examine herein the dependence of skin penetration on the chemical properties of ILs. First, the impact of ion stoichiometry on skin penetration of drugs was assessed using CAGE as a representative IL, and it showed that a molar ratio of 1:2 of choline to geranic acid in CAGE yielded the highest delivery. Subsequently, variants of CAGE were prepared using anions with structural similarity to geranic acid and cations with structural similarity to choline. The cation to anion ratio was held constant at 1:2 for all variants. A range of permeation enhancement effects was observed among the CAGE variants with some variants outperforming the original CAGE composition. Mechanistic studies revealed that the potency of ILs in enhancing transdermal drug delivery correlated inversely with the inter-ionic interactions as determined by 2D NMR. Using this understanding, a new CAGE variant was designed, and it provided the highest delivery of ruxolitinib of all ILs tested here. Overall, these studies provide a generalized framework for optimizing ILs for enhancing skin permeability.

### Introduction

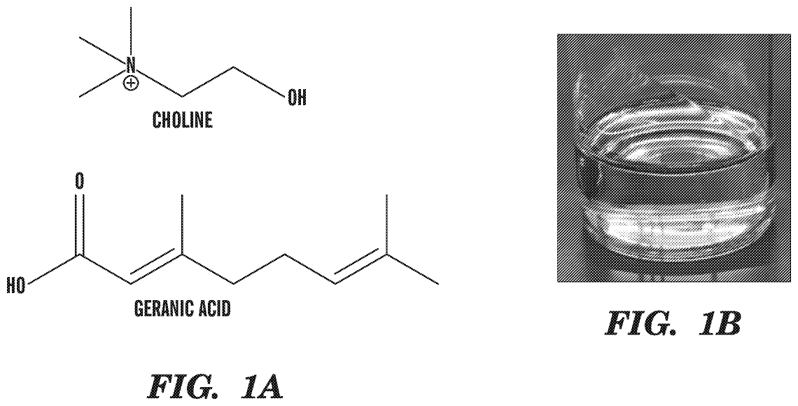

Transdermal drug delivery offers a painless mode of administering drugs while concurrently avoiding first-pass metabolism. [1] However, overcoming the uppermost transport barrier of skin, the stratum corneum (SC), is a significant challenge.[1] The SC consists of a tightly packed brick-and-mortar-like structure of corneocytes, surrounded by lipids [2]. Only few drugs possessing low mass and high lipophilicity are capable of navigating the SC barrier without assistance. Several methods have been engineered to enhance skin permeability to drugs including the use of ultrasound, [3,4] microneedles, [5,6] iontophoresis, [7,8] jet injectors, [9,10] and permeation enhancers, [11-13] among others. Recently, ionic liquids and deep eutectic solvents (hereafter collectively referred to as ionic liquids (ILs) for simplicity) have shown great promise for transdermal drug delivery.[14-19] We have previously demonstrated that a choline and geranic acid based IL, referred to as CAGE (FIG. 1), has shown to be effective in transdermal delivery of insulin [20] as well as small molecule antimicrobial agents.[17]

The efficacy of ILs in enhancing skin permeability is expected to depend on their ionic constituents and composition. However, such knowledge-base is critically missing from the literature. One study has shown that the efficacy of CAGE in insulin delivery depends strongly on its ion stoichiometry. The study further demonstrated that CAGE mediates its enhancement effect primarily through its interaction of SC lipids.[16] However, the generalized understanding of the role of ion properties in determining IL's delivery efficacy is still lacking.

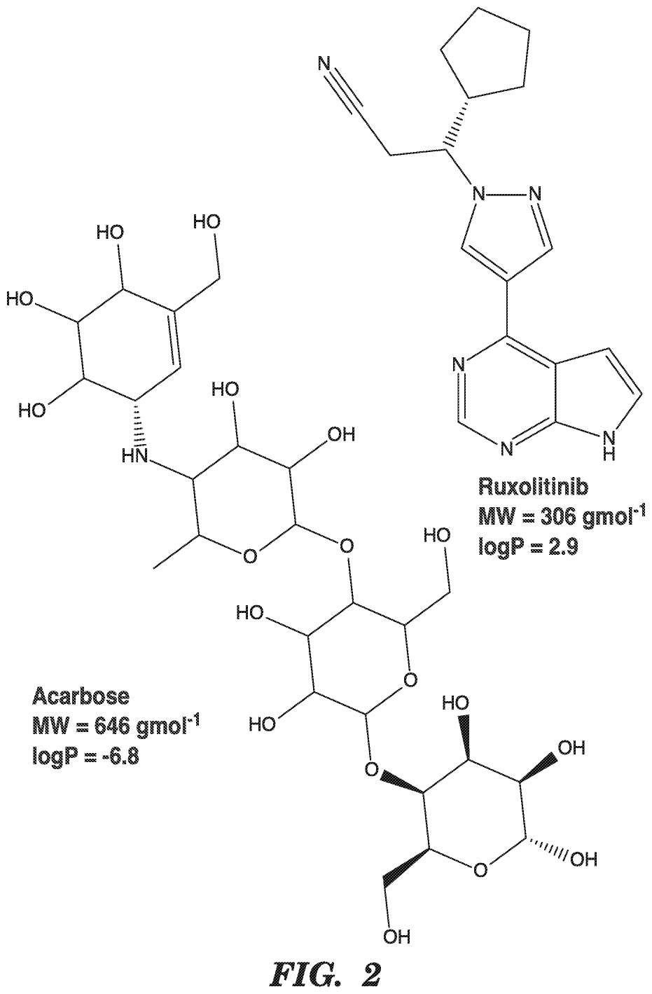

The work presented herein represents the first systematic study on the role of anion and cation properties in IL-mediated transdermal drug delivery. Drugs of differing hydrophobicities were used to assess the transport potential of the ILs (FIG. 2). The first drug, acarbose, is a hydrophilic molecule used to treat Type 2 diabetes by inhibiting enzymes that digest carbohydrates. [27] It is currently administered orally but has a very low bioavailability and a high gastrointestinal side effect profile and therefore has limited use. [28] The second drug, ruxolitinib is a hydrophobic janus kinase (JAK) inhibitor that is currently FDA approved as an oral pill for use in myelofibrosis. [29,30] It has also shown promise in treating alopecia, therefore its topical delivery is of interest. [31,32] The ILs used in this study were built around the composition of CAGE. Specifically, eight organic acids of varying molecular weights, pKas and hydrophobicities were used as alternatives to geranic acid, and seven organic quaternary amines were used as alternatives to cholines. The resultant ILs were characterized by NMR and their ability to deliver ruxolitinib and acarbose was measured in vitro.

Methods and Materials

Materials

Choline bicarbonate, geranic acid, the tertiary amines, halogenated alcohols, Phosphate Buffered Saline (PBS), D20 and deuterated DMSO were obtained from Sigma Aldrich (St. Louis, Mo., USA). The commercial geranic acid (85%, yellow liquid) was purified prior to use with at least five successive freeze-thaw cycles with acetone until the solution was clear. The residual acetone was removed under reduced pressure prior to use. Porcine skin (Lampire Biological Laboratories, Pipersville, Pa.) was kept at −80′C and thawed immediately prior to use.

Methods

Synthesis of CAGE. CAGE was synthesized at various ion stoichiometries as previously reported. [17] Choline bicarbonate and geranic acid was mixed at various molar ratios including 1:4, 1:2, 1:1 and 2:1 to prepare CAGE by salt metathesis reaction. Each CAGE composition was characterized via NMR with DMSO-d₆ on an Agilent DD2™ 600 MHz spectrometer.

Synthesis of Choline Analogs. Seven variants of choline were prepared to assess the dependence of transport on cation properties. The synthesis was modelled on that reported in Ref [33]. The starting material for all variants was either a triethylamine, tripropylamine or tributylamine. In each case, the amine was reacted with a halogenated alcohol in a 1:1 molar ratio by dissolution in toluene (30 mL) and heating in a round bottom flask for 12 hours at a pre-determined temperature Table 4). The various reactants, the reaction temperatures, and key information about the resulting products can be found in Table 4. A majority of the solvent was then removed using a rotary evaporator at 20 mbar at 60° C. for 2 hours. The residual solvent was removed under reduced pressure at 60° C. for 48 hours.

ILs of Geranic Acid with Choline Alternatives. Quaternary amines were reacted with recrystallized geranic acid in a 1:2 molar ratio at 40° C. for 12 hours, similarly to the CAGE synthesis reported previously.[34] A rotary evaporator was then used to dry the resulting ionic liquids at 20 mbar at 60° C. for 2 hours. The residual water was removed under reduced pressure at 60° C. for 48 hours.

ILs with Geranic Acid Alternatives. Carboxylic acids dissolved in the minimum amount of ultrapure water (or methanol in the case of salicylic acid) needed for dissolution (<5 mL) were reacted with choline bicarbonate in a 1:2 molar ratio (choline: carboxylic acid) at 40° C. for 12 hours. A rotary evaporator was then used to dry the resulting ionic liquids at 20 mbar at 60° C. for 2 hours. The residual water was removed under reduced pressure at 60° C. for 48 hours.

NOESY measurements. Samples were prepared by placing dried IL into an NMR tube alongside a coaxial insert containing deuterated DMSO. An Agilent DD2TM 600 MHz spectrometer was used. A 90° pulse width of 11.25 was employed, number of increments (ni) was set to 128 and the number of scans was set to 4. The spectra were phase corrected after measurement.

Skin Penetration Studies. The skin penetration studies were undertaken using porcine skin in Franz diffusion cells, as described in full previously (skin area=1.7 cm²). [35] Briefly, thawed, washed porcine skin was placed in a diffusion cell with the SC facing upwards. The acceptor component of the cell was filled with PBS and equipped with a magnetic stirrer bar. Only cells with a measured trans-epidermal current less than 12 μA were used in the study and the treatments were randomly assigned. 300 μL of drug solution in each IL (1 mg/mL) was placed on top of the skin, ensuring full coverage. The cell was placed on a stirrer plate at 37° C. for 24 hours, at which point the skin was removed and the surface was washed gently with PBS. Each skin layer was then separated. The stratum corneum was removed by tape stripping (up to ten layers), the epidermis was separated from the dermis with a scalpel, and a 4 mm punch was used thrice to remove a third of the dermis (by area). The tissue was then placed into either methanol (ruxolitinib) or a 50% methanol/PBS mixture (acarbose), and left to shake overnight to extract the drug, which was then analyzed by HPLC, as described below.

HPLC Analysis. The analysis of the amount of drug present in each layer was determined by HPLC. In each case a calibration curve was prepared, consisting of ten samples covering a concentration range of 2×10⁻³-1 mg/ml. For acarbose, a 250×4.6, 5 μm Nucleosil-NH2 column was used. The isocratic mobile phase was 1 mL/min acetonitrile:phosphate buffer (where 1 L contained 0.6 g of KH₂PO₄ and 0.48 g of Na₂HPO₄) in a ratio of 76:24, and the UV detector (at 210 nm) showed a peak which appeared at 5.2 minutes. For ruxolitinib, an Inertsil ODS C-18 column with 250×4.6 mm internal diameter and 5μm particle size was employed. A mobile phase of isocratically eluted THF: Methanol: Acetonitrile 10:40:50 (v/v/v) with a flow rate of 1 mil/min was used, and the peak at 3.2 minutes was identified as the product peak (detected by UV at 270 nm).

Results

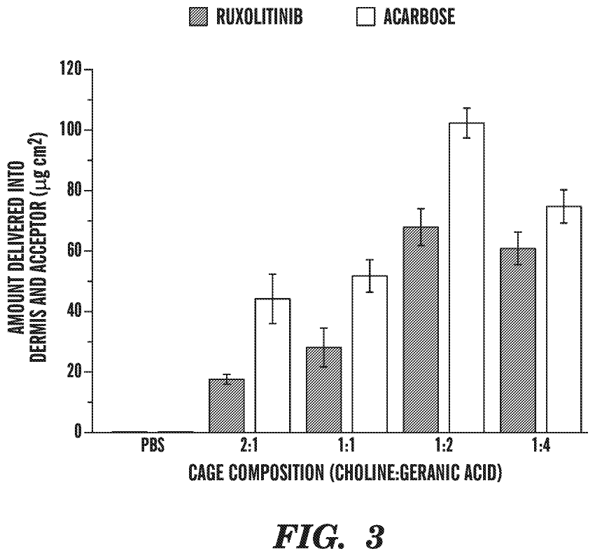

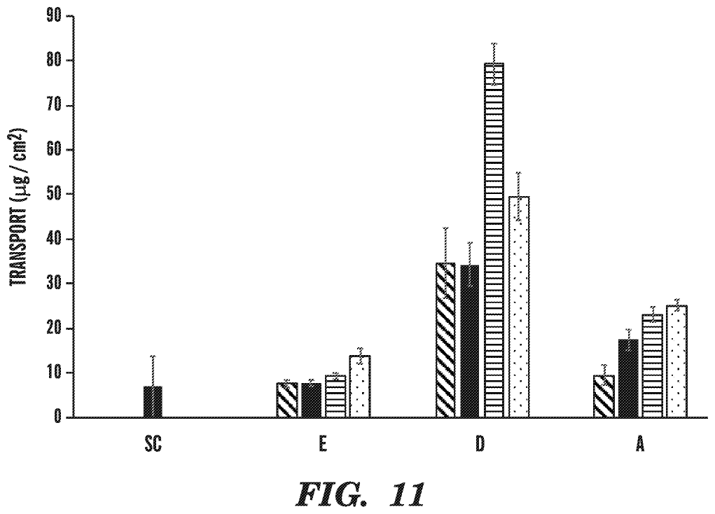

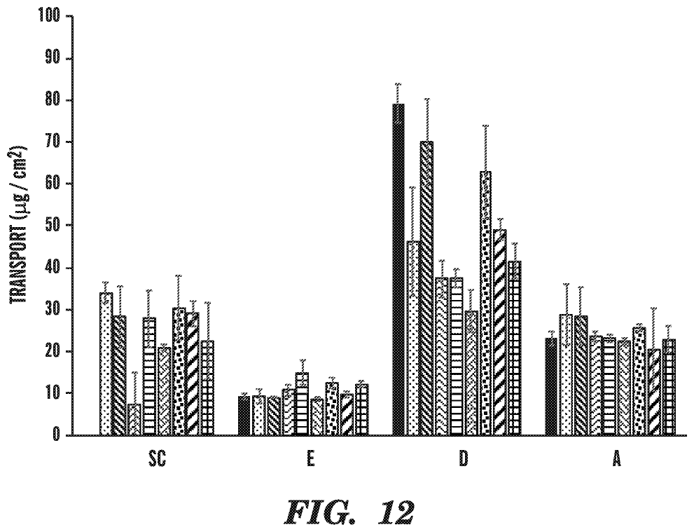

Dependence of Delivery Efficacy on Ion Stoichiometry. Initial studies were performed to assess the role of ion stoichiometry in CAGE in determining its ability to deliver acarbose and ruxolitinib. Four variants of CAGE were synthesized with choline:geranic acid ratios of 2:1, 1:1, 1:2, and 1:4. Acarbose and ruxolitinib were dissolved in CAGE at a concentration of 1 mg/ml. Both drugs dissolved completely in all CAGE variants at this concentration. Delivery of both drugs into skin exhibited a strong dependence on CAGE composition (FIG. 3, acarbose (red) and ruxolitinib (blue)). Amounts shown in FIG. 3 correspond those delivered into dermis and acceptor compartment. Detailed distribution of drugs in various skin layers is shown in FIGS. 11 and 12. Highest amounts of delivered drugs were found in the dermis, followed by acceptor, followed by the epidermis. No significant transport of either drug was observed from PBS (negative control). Acarbose was soluble in PBS at a concentration of 1 mg/ml, whereas ruxolitinib was soluble only at a concentration of 0.2 mg/ml and hence a saturated solution in PBS was used in the donor. In case of ruxolitinib, the delivery efficacy increased by ˜4-fold as the geranic acid content increased by 2-fold (2:1 CAGE to 1:2 CAGE), after which it plateaued. A similar trend was observed in case of acarbose, thus confirming that 1:2 CAGE exhibits the highest delivery efficacy for hydrophilic as well as lipophilic drugs. This is consistent with previous study with insulin, whereby 1:2 CAGE performed the best out of the four variants presented. [16] Note that no significant transport of ruxolitinib or acarbose was observed when they were solubilized in pure geranic acid or 80% choline bicarbonate in water. Given that the 1:2 choline:geranic acid ratio provided maximum delivery regardless of the drug hydrophilicity, subsequent variants were prepared at this ion stoichiometry.

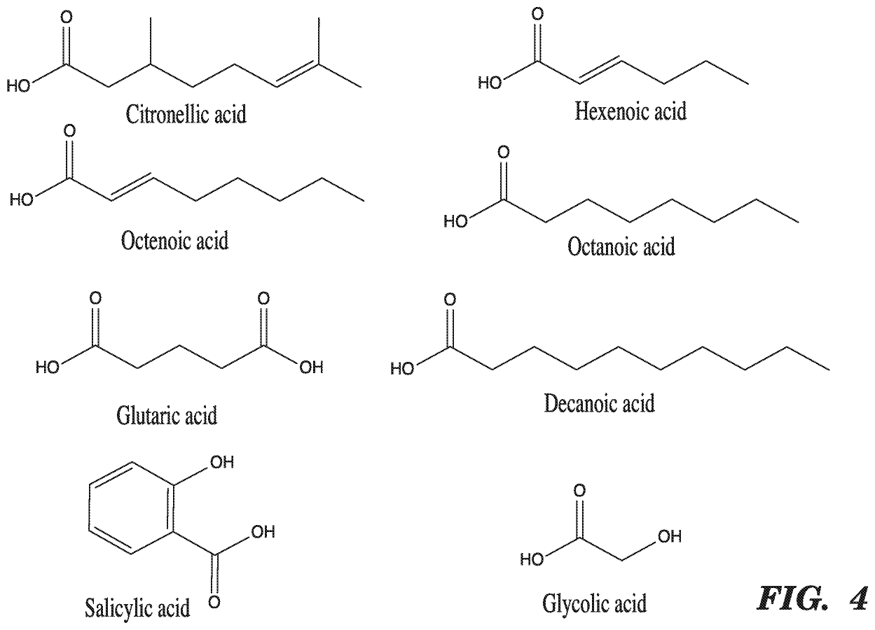

Dependence of Delivery Efficacy on Anion Chemistry. A first set of IL variants was synthesized by replacing geranic acid with other organic acids. Eight alternatives to geranic acid were used (FIG. 4) The first, and the closest variant of geranic acid was citronellic acid, which possesses one less double bond compared to geranic acid and is otherwise identical. Octanoic acid possesses the same number of carbons in the main backbone as geranic acid but lacks unsaturation and the methyl side groups present in geranic acid. Octenoic acid is an unsaturated analog of octanoic acid and provides an opportunity to assess the role of unsaturation in delivery efficacy. Decanoic acid possess an identical number of total carbons as geranic acid and lacks unsaturation, thus providing an opportunity to assess the role of total number of carbons in the delivery efficacy. Glutaric acid is the only dicarboxylic acid included in the list, and salicylic acid was the only aromatic acid. Some of the key properties of anions including molecular weight pKa, Log P and number of carbon atoms are listed in Table 1.

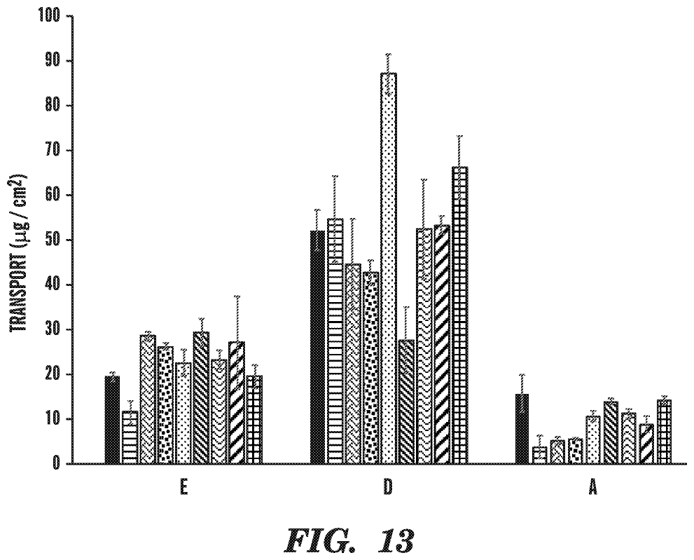

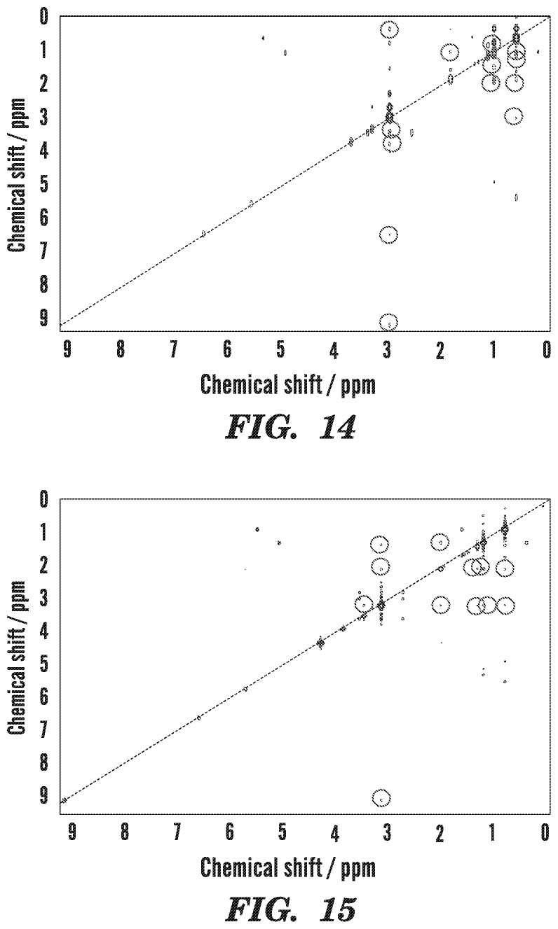

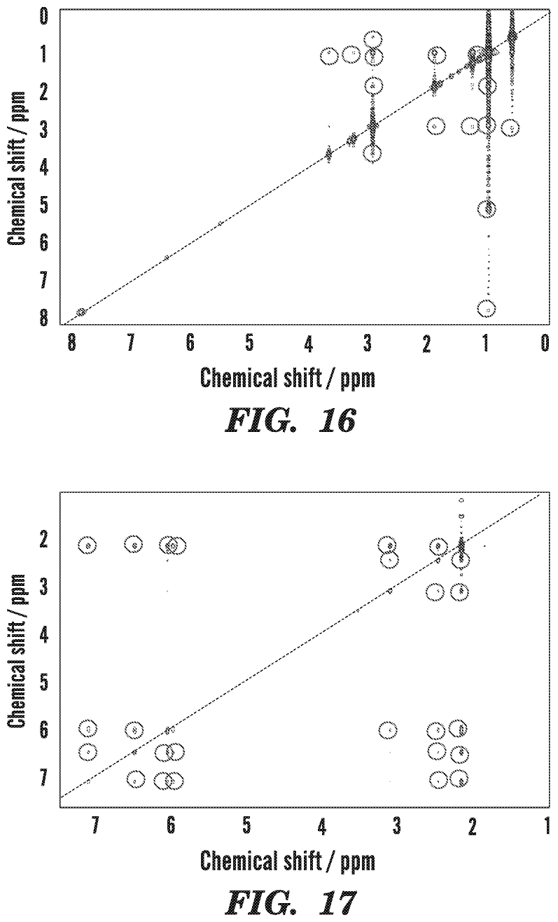

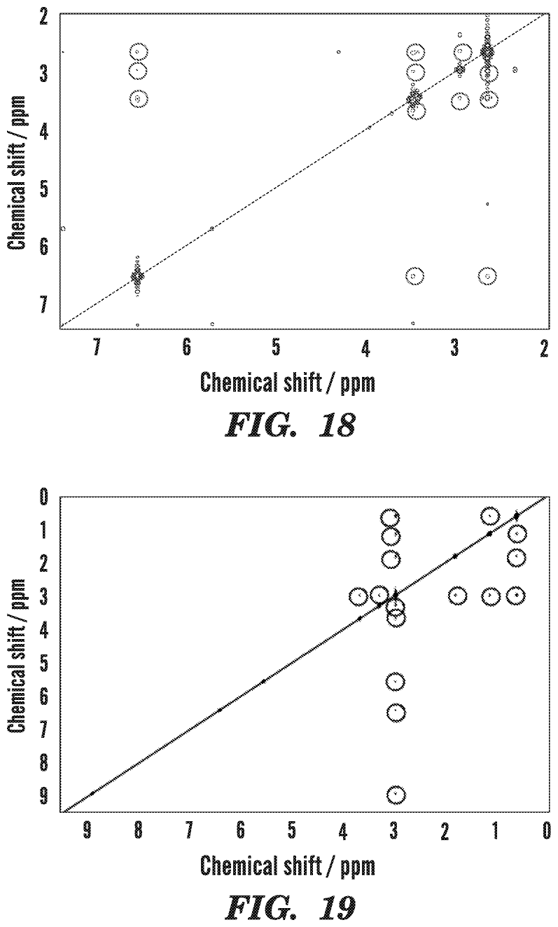

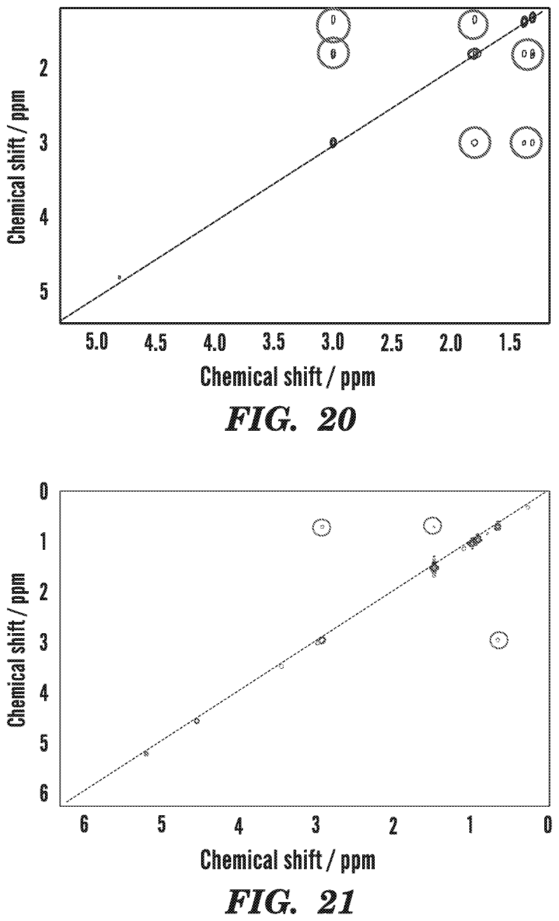

Ionic liquids of each of these carboxylic acids were synthesized using choline as a counterion at a stoichiometric ratio of 1:2 choline:acid using salt metathesis of choline bicarbonate with the acid. Ruxolitinib and acarbose were dissolved in each IL variant at a concentration of 1 mg/ml. All ILs exhibited enhanced delivery of acarbose and ruxolitinib into the skin compared to the control (PBS). In general, most of the drug was found in the dermis, followed by the epidermis, then the acceptor (FIGS. 13 and 14). The amount of acarbose and ruxolitinib delivered exhibited a significant dependence on IL chemistry (Table 2), particularly the amount of drug delivered to the dermis. In case of acarbose, geranic acid outperformed other anions. Hexenoic and glycolic acid ranked second and third, both are anions with low Log P. Note, however, that glutaric acid, also with a low Log P of 0.05 did not perform well, suggesting that the enhancement is not simply a function of lipophilicity. Interestingly, the same two anions, glutaric acid and hexenoic acid, were also among the least effective anions for the delivery of ruxolitinib. Citronellic and octanoic acid outperformed geranic acid in terms of delivery of ruxolitinib. An overall ranking of all variants was determined by averaging their ranking for delivery of ruxolitinib and acarbose to identify ILs which provide permeation enhancement across a broad spectrum of drugs.

Geranic acid ranked highest followed by its close analog citronellic acid. Small anions including salicylic acid and glutaric acid were on the lower end of the ranking spectrum. Interestingly, decanoic acid, which possesses an identical number of carbons as geranic acid ranked very low and was poorly effective for acarbose as well as ruxolitinib. Octanoic acid, which possesses the same number of carbons as those in geranic acid backbone performed fairly well, ranking behind geranic acid and citronellic acid. Octenoic acid ranked just below octanoic acid, similar to citronellic acid ranking below geranic acid, thus indicating that a single unsaturated bond makes an impact on delivery efficacy.

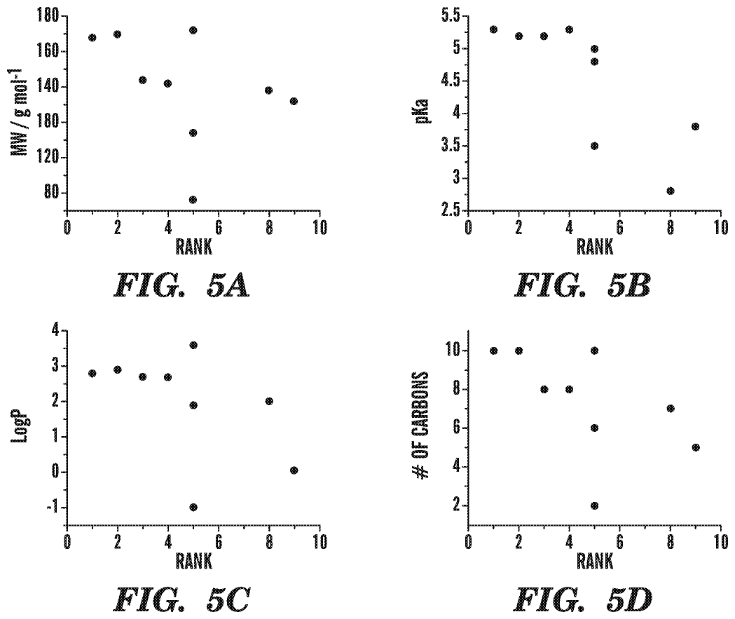

Correlation between Delivery Efficacy and Molecular Parameters. It was sought to assess whether the transport ranking of anions correlated with their physicochemical parameters including molecular weight, pKa, Log P, and the number of carbons (FIGS. 5A-5D). These parameters were selected because they capture some of the key chemical descriptors. No significant, quantitative trends emerged from these plots. Some qualitative observations, however, could be seen. For example, best ranking anions possessed pKa values of over 5 (FIG. 5B) and log P values close to 3 (FIG. 5C), implying that ILs prepared from weak hydrophobic acids are more effective in enhancing skin permeability. Given that there is no simple correlation between single physico-chemical properties of the acids and their transport efficacies, other possibilities were explored.

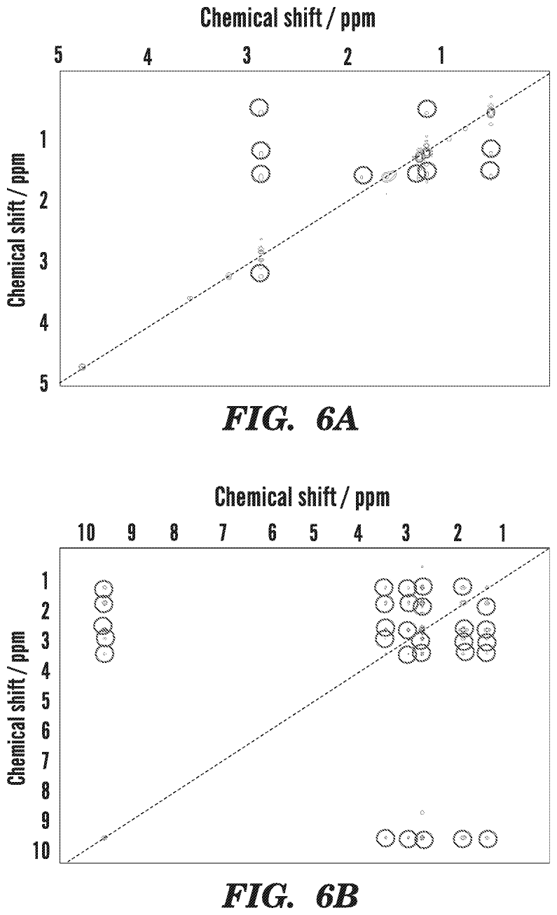

One of the critical parameters that must be considered when employing an IL is the interaction between its ionic components. Specifically, strong interactions between the anions and cations are critical to the formation and stability of the IL. However, the interactions between the IL components and the SC lipids are also critical in determining their efficacy of enhancing skin permeability. Given the relevance of the interactions between the anion and cation and given that the properties of the anion alone did not correlate with the measured permeation ranking, it was sought to determine whether the intra-ion interactions correlate with the overall permeation ranking. To this end, we investigated the intra-ionic interactions using 2D Nuclear Overhauser Effect SpectroscopY (NOESY). [36] This technique plots the NMR data on two frequency axes and uses the diagonal peaks and cross-peaks to reveal spatial interactions between the underlying chemical groups (in this case, anion and cation) by counting the number of cross-peaks (FIGS. 6A, 6B). Each cross peak represents two groups of protons that are within 5 nm of each other. Due to its unique ability to resolve spatial interactions, NOSEY has been used to study structures of proteins, [37,38] peptides, [39,40] and ionic liquids. [36,41,42]

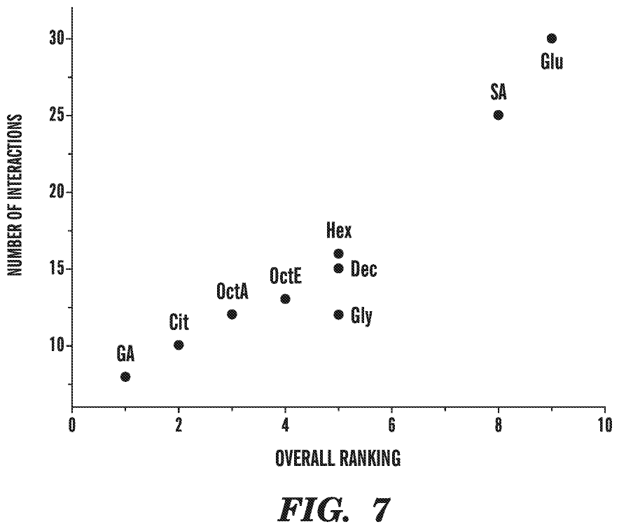

Exemplar NOESY spectra are shown in FIGS. 6A-6B, with the in-phase cross-peaks circled, which indicate the parts of the ions that are within 5 nm of each other. The remaining spectra are presented in SI. The number of measured cross peaks varied over a wide range. For example, ten peaks were found for choline: citronellic acid (FIG. 6A) and thirty for choline: glutaric acid (FIG. 6B). The overall transport ranking of ILs exhibited a strong correlation with the number of NOSEY cross peaks (FIG. 7). Geranic acid and citronellic acid, which possessed among the lowest number of cross peaks, exhibited highest transport rankings whereas glutaric and salicylic acids, which possessed among the highest number of cross peaks, exhibited the lowest transport ranking.

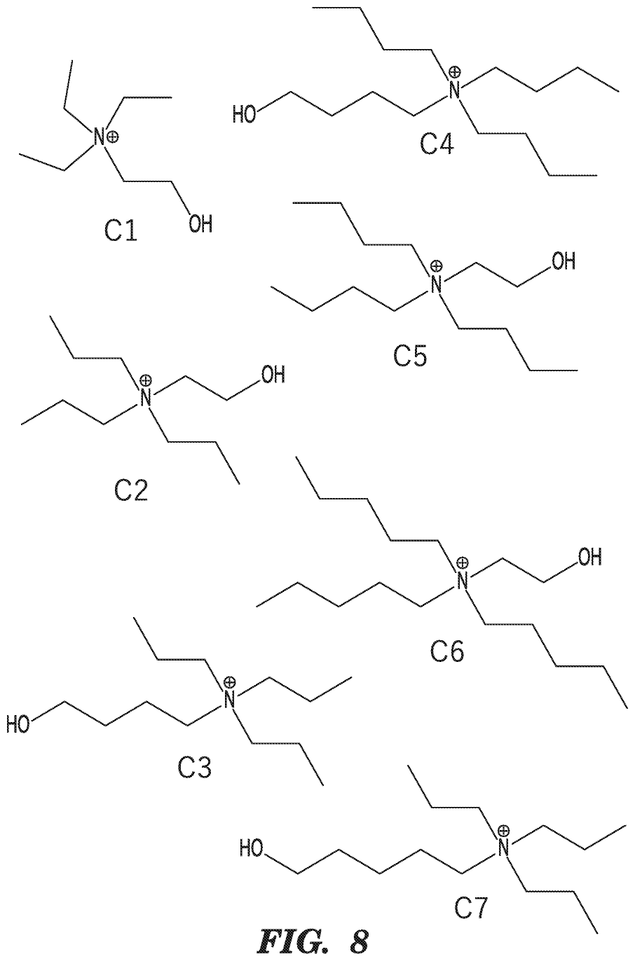

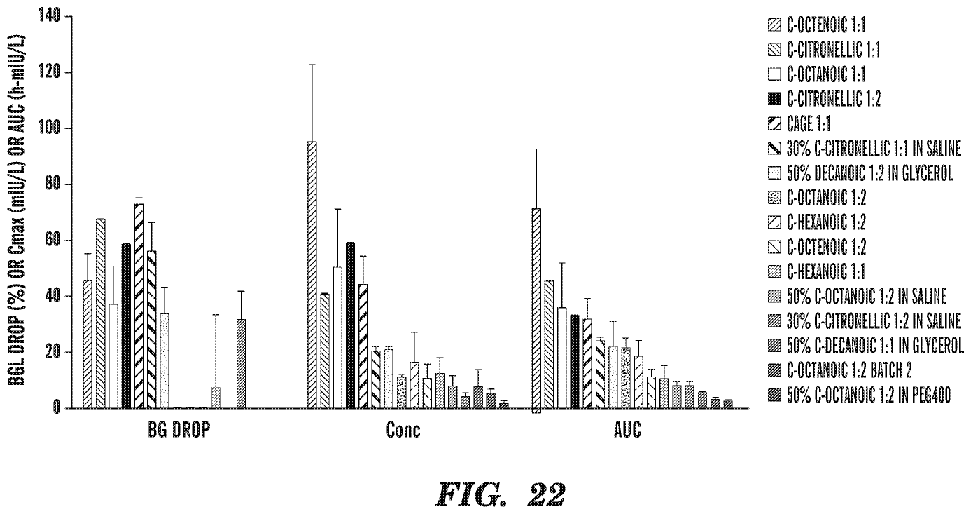

Modification of Cations. Next, modification of the cation was undertaken by increasing the alkyl head groups of choline and lengthening the ethoxy chain. A total of 7 cations were synthesized in addition to choline (FIG. 8). Ionic liquids of each cation were synthesized using geranic acid as an anion at a ratio of 1:2 (geranic acid: cation) and characterized using NMR. Ruxolitinib and acarbose were dissolved in each IL at a concentration of 1 mg/ml and their delivery into skin was measured. The ability of the ILs prepared with various cations to deliver acarbose and ruxolitinib is shown in Table 3. In case of the acarbose, little variation was seen across the cations studied and none of the newly synthesized cations outperformed choline. This is likely a result of the hydrophilic nature of acarbose and because the choline cation is the most hydrophilic cation in the study, it may render more hydrophilicity to the IL. In case of ruxolitinib, a significant dependence of transport on the cation was observed. Bulkier cations, for example, C6 and C7 performed significantly better than choline in terms of the amount delivered. Many newly synthesized choline alternatives performed better than choline and C1 was ranked first among them. Once again, this improved performance correlated with NOSEY spectrum. Specifically, C1: geranic acid IL exhibited only three cross peaks compared to eight in the case of choline: geranic acid (FIG. 21). The added bulk of the substituents in C₁ is hypothesized to reduce close contact between the cation and the geranic acid.

Simultaneous Changes in Anion and Cation

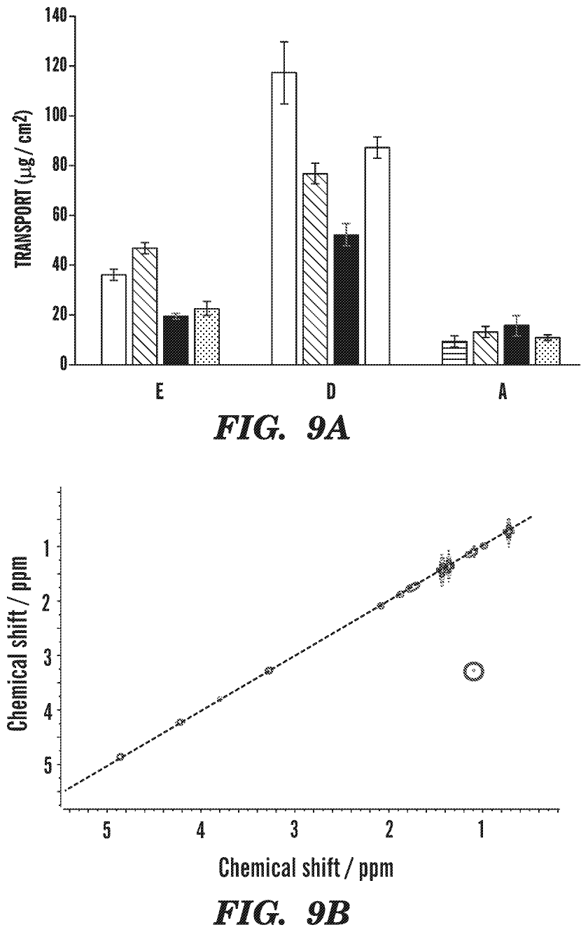

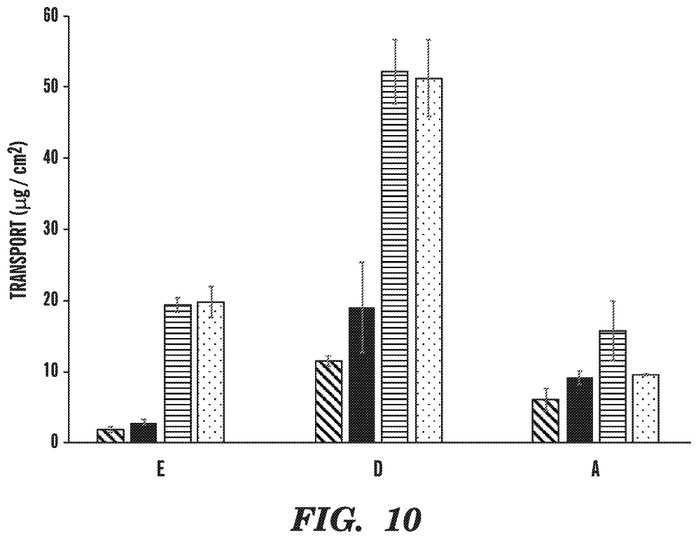

Having explored several anion and cation alternatives and having established a correlation between the transport ranking and NOSEY cross-correlation peaks, we combined two anions and two cations to make representative ILs. The two anions were geranic acid and its best alternative, citronellic acid. The two cations were choline and its best alternative, C1. Four ILs were compared; choline: geranic acid (CAGE), choline: citronellic acid, C1: geranic acid and C1: citronellic acid. Delivery of ruxolitinib into the skin was measured with all ILs. NOSEY spectrum for all ILs was also measured. A significant dependence of ruxolitinib delivery on IL composition was found (FIG. 9A). C1: citronellic exhibited the highest delivery with 117.1±12.7 μg cm⁻²ruxolitinib measured in the dermis and acceptor after 24 hours. In comparison, C1: geranic acid, choline: geranic acid and choline: citronellic acid delivered 89.9±4.6, 68.0±6.1, and 97.8±4.6 μg cm⁻². The delivery efficacy once again exhibited a strong correlation with NOSEY cross correlation peaks, with C1: citronellic acid exhibiting only one peak compared to CAGE exhibiting 8 (FIG. 9B).

### DISCUSSION

Ionic liquids have recently shown great promise in facilitating transdermal transport of pharmaceuticals.[14,43] In particular, imidazolium,[18] quaternary ammonium,[44,45] and cyclic onium-based[43] cations have been investigated ex vivo with respect to their ability to transport small molecules through intact stratum corneum to the dermis. There is currently still, however, limited physical understanding of the factors that yield optimal ILs for transdermal delivery, particularly for quaternary ammonium-based ILs.

The studies reported herein present a systematic evaluation of the dependence of transdermal drug delivery on anion and cation properties for choline carboxylic acid-based ionic liquids. We first evaluated the dependence of transport on the ion stoichiometry using CAGE as an exemplar IL. The results showed that ILs with excess anion (geranic acid) exhibited higher transport than those with equimolar or cation-heavy ILs for ruxolitinib as well as acarbose delivery. These conclusions are consistent with those reported before using insulin [16]. The results presented here, taken together with previous studies on insulin, ceftazidime and mannitol, show that CAGE can deliver a wide range of molecules (from a MW of 180 for mannitol to 6000 for insulin, from a Log P of −6.8 for acarbose to 2.9 for ruxolitinib, and various molecule types including saccharides, peptides and aromatics). The ability of CAGE to enhance a wide variety of molecules indicates that the primary effect of CAGE is on the SC, thus addressing the primary barrier function.

Fourier Transform Infrared Spectroscopy (FTIR) studies have shown that CAGE impacts the SC lipids as measured by the reduction of the methylene peaks, indicative of lipid extraction and/or fluidization.[16] FTIR studies conducted in the literature using imidazolium-based ionic liquids are also consistent with our finding of the impact of ionic liquid on the lipid-rich SC. [18] CAGE variants with higher proportion of geranic acid (>50 mol %) produced a more significant impact on SC lipids, which is consistent with the observed trends of molecular fluxes. Interestingly, there is a clear optimum for the geranic acid concentration in the formulation since pure geranic acid did not yield a noticeable skin flux of either drug. This likely originates from the high lipophilicity of geranic acid, which while beneficial for solubilization of ruxolitinib, is likely to reduce its partitioning into the skin. Similar effects of the adverse effects of formulation lipophilicity on delivery of lipophilic drugs have been previously reported for other chemicals. [46] Choline thus appears to mitigate the lipophilicity of the anion alone and enable portioning of geranic acid as well as the drug into the skin.

Several novel variants of CAGE were prepared at a fixed stoichiometry of cation: anion of 1:2. In one set of variants, the cation was kept the same in the form of choline whereas eight carboxylic acid anions were used. No clear dependence of drug transport on any single anion parameter including molecular weight, pKa, Log P and number of carbons was observed. Qualitatively, large hydrophobic anions ranked better than small hydrophilic anions. Specifically, hydrophobic tails of anions and/or cations play a key role in fluidization of the SC lipids. Since choline is highly hydrophilic and lacks aliphatic chains, anions provide the primary mode of lipid disruption. Geranic acid and citronellic acid, two of the most hydrophobic anions in this study, emerged as among the most effective. Decanoic acid was an exception to this trend. In spite of possessing comparable MW and pKa, and a slightly higher Log P compared to geranic acid, decanoic acid did not perform well. This suggests a strong role of unsaturation in determining the delivery efficacy. Studies in the chemical enhancer literature have previously pointed to the role of unsaturation in transdermal flux enhancement. Specifically, increased unsaturation was hypothesized to enhance SC lipid disruption likely due to increased steric constraints posed by unsaturation. [49]

It is contemplated herein that two factors dominate the design of an ideal ionic liquid for transdermal delivery; potency of the anion and its ability to enter the skin. The potency of the anion relates to how effectively it is inherently able to disrupt the lipids in the SC in order to facilitate drug transport. Anion's ability to enter the skin may impact its ability to come in contact with the SC lipids. Extensive intra-ionic interactions could reduce the anion's ability to enter the SC. Fatty acids have shown high efficacy in skin permeation enhancement,[50,51] and the relative abilities of fatty acids to enter the skin can significantly impact the efficacy of ILs.

A strong correlation was found between the number of cross-peaks in NOSEY and the overall permeability ranking. The cross-correlation peaks in NOSEY spectra exhibited a wide range, much greater than that measured for any single molecular parameter. The number of peaks varied from one for C1: citronellic acid to 30 for C1: glutaric acid. Fundamentally, the number of cross peaks indicates the intramolecular interactions between the ions that are mediated by protons. That is, each in-phase peak (the same color as the 1D diagonal line) indicates that molecules are within 5 nm of each other as an average across the liquid. 2D NMR has previously been employed successfully in neat ionic liquids to investigate inter-ionic interactions in ILs.[36,41,42] Previous NOSEY studies with CAGE have also indicated the presence of interactions between choline and geranic acid, and that these interactions vary with the ion stoichiometry.[16]

Without wishing to be bound by theory, it is contemplated herein that the intra-ion interactions in ILs play an important role in determining skin transport in at least two ways. First, the presence of strong intra-ion interactions can create supramolecular structures that are either large in size and/or energetically stable which reduces their entry into SC and subsequent disruption of SC lipids. Molecular dynamic simulation studies with CAGE have indicated strong hydrogen bonding between choline and geranic acid,[52] with primary interactions arising from those between the carboxyl group of geranic acid and hydroxyl group in choline. In particular, since the lipid-disruptive effect of ILs is primarily mediated by the hydrophobic anion, interactions that favor solubilization and retention of anions in the formulation will reduce their ability to enter the skin and mediate skin permeation. Second, it is possible that the ILs that exhibit high level of interactions among their own ionic constituents also interact strongly with the solvated drug and reduce its partitioning into the skin. Regardless of its mechanistic origin, the correlation between the transport efficacy ranking and cross-peaks is quite striking and is observed not only for the anionic variants of CAGE but also for combined cationic-anionic variants. Specifically, C1: citronellic acid, which exhibited the fewest number of cross peaks (one), exhibited the highest delivery of ruxolitinib.

It is possible that the presence of a bulky aliphatic group like in C1 restricts the interactions of its hydroxyl groups with the carboxyl group in citronellic acid. In other words, bulky hydrophobic cations and large hydrophobic anions make can potentially make excellent ILs for transdermal drug delivery. The studies presented here clearly demonstrate that 2D NMR can be used as a screen to determine the potency of ILs in enhancing skin permeability.

Among the anions studied here, several have a history of use in humans. Among the best performing anions besides geranic acid, citronellic acid is used in cosmetic applications as an antidandruff and masking agent. Octanoic acid (caprylic acid) is a commonly used dietary supplement. The modified choline molecules have some history of use in industrial applications, namely in catalysis.[33] C1 has also been used in perfusion studies in rats examining the blood-brain barrier, but no comprehensive toxicity screening has been undertaken. Promisingly, choline-based cations have shown acceptably low toxicity, particularly in comparison to imidazolium-based cations.[53]

### CONCLUSIONS

The study provides a framework for the design and screening of ionic liquids for transdermal drug delivery. Using two model drugs, acarbose and ruxolitinib, and 16 ionic liquids, we studied the dependence of transport on ion stoichiometry as well as molecular composition. In case of CAGE a composition comprising 1:2 choline: geranic acid yielded maximum transport enhancement. Systematic modification of the anion revealed that the ILs with the fewest inter-ionic interactions were most successful at transdermal transport, and it is contemplated herein that this is a result of the ability of the anions to freely enter the skin. Modifications of cations that further reduced the inter-ionic interactions also improved delivery efficacy. Combination of the most effective cations and anions led to novel ILs that provided highest enhancement of ruxolitinib transport. This work represents the first systematic structural study of ILs in transdermal drug delivery, and provides a paradigm for designing new, effective topical formulations.

TABLE 1
Molecular properties of the carboxylic acids used
to create the new IL. LogP and pKa were theoretically
determined as described in Ref. [54]

| Name | MW | pKa | LogP | Number of Cs |
|---|---|---|---|---|
| Geranic Acid | 168.2 | 5.3 | 2.8 | 10 |
| Citronellic Acid | 170.2 | 5.2 | 2.9 | 10 |
| Decanoic Acid | 172.3 | 5.0 | 3.6 | 10 |
| Glutaric Acid | 132.1 | 3.8 | 0.05 | 5 |
| Glycolic Acid | 76.1 | 3.5 | −1 | 2 |
| Hexenoic Acid | 114.1 | 4.8 | 1.9 | 6 |
| Octanoic Acid | 144.2 | 5.2 | 2.7 | 8 |
| Octenoic Acid | 142.2 | 5.3 | 2.7 | 8 |
| Salicylic Acid | 138.1 | 2.8 | 2.0 | 7 |

TABLE 2
Transport of acarbose and ruxolitinib to the dermis and
acceptor over 24 hrs by a range of ionic liquids with
modified anions (N = 3, Error bars indicate SEM).

| | Acarbose | | Ruxolitinib | | |
|---|---|---|---|---|---|
| | Amount | | Amount | | |
| | delivered | | delivered | | |
| | to dermis | | to dermis | | |
| | and acceptor | | and acceptor | | |
| | in 24 hours | | in 24 hours | | Average |
| Anion used | (μg cm⁻²) | Rank | (μg cm⁻²) | Rank | Rank |
| Geranic Acid | 102.4 ± 4.9 | 1 | 68.0 ± 6.1 | 3 | 1 |
| (GA) | | | | | |
| Citronellic Acid | 75.1 ± 14.8 | 4 | 97.8 ± 4.6 | 1 | 2 |
| (Cit) | | | | | |
| Octanoic Acid | 64.6 ± 5.2 | 6 | 80.5 ± 7.0 | 2 | 3 |
| (OctA) | | | | | |
| Octenoic Acid | 69.7 ± 10.1 | 5 | 62.3 ± 2.7 | 5 | 4 |
| (OctE) | | | | | |
| Decanoic Acid | 61.0 ± 4.5 | 7 | 63.6 ± 11.1 | 4 | 5 |
| (Dec) | | | | | |
| Glycolic Acid | 88.5 ± 11.1 | 3 | 48.3 ± 2.8 | 8 | 5 |
| (Gly) | | | | | |
| Hexenoic Acid | 98.4 ± 12.4 | 2 | 41.6 ± 7.4 | 9 | 5 |
| (Hex) | | | | | |
| Salicylic Acid | 60.7 ± 2.3 | 8 | 58.5 ± 9.9 | 6 | 8 |
| (SA) | | | | | |
| Glutaric Acid | 52.1 ± 5.1 | 9 | 49.9 ± 10.1 | 7 | 9 |
| (Glu) | | | | | |

TABLE 3
Transport of acarbose and ruxolitinib to the dermis and
acceptor over 24hrs by a range of ionic liquids with
modified cations (N = 3, Error bars indicate SEM).

| | Acarbose | | Ruxolitinib | | |
|---|---|---|---|---|---|
| | Amount delivered | | Amount delivered | | |
| | to dermis | | to dermis | | |
| | and acceptor | | and acceptor | | |
| Cation | in 24 hours | | in 24 hours | | Average |
| used | (μg cm⁻²) | Rank | (μg cm⁻²) | Rank | Rank |
| Choline | 102.4 ± 4.9 | 2 | 68.0 ± 6.1 | 8 | 5 |
| C1 | 92.1 ± 3.4 | 3 | 89.9 ± 4.6 | 3 | 1 |
| C2 | 85.0 ± 2.1 | 4 | 62.0 ± 6.4 | 9 | 6 |
| C3 | 83.8 ± 5.5 | 6 | 72.4 ± 4.5 | 7 | 6 |
| C4 | 104.5 ± 8.1 | 1 | 80.8 ± 7.3 | 6 | 2 |
| C5 | 72.1 ± 4.1 | 8 | 80.1 ± 4.5 | 5 | 6 |
| C6 | 77.5 ± 2.1 | 7 | 107.2 ± 8.7 | 1 | 4 |
| C7 | 84.9 ± 18.8 | 5 | 92.7 ± 6.5 | 2 | 2 |

TABLE 4
Reaction conditions for the cationic precursors.

| Cation | | Halogenated | Temperature | |
|---|---|---|---|---|
| Number | Tertiary Amine | Alcohol | (° C.) | Comments |
| 1 | Triethylamine | 2-chloroethanol | 100 | |
| 2 | Tripropylamine | 2-chloroethanol | 80 | |
| 3 | Tripropylamine | 4-chlorobutanol | 70 | Dark Red |
| | | | | Liquid |
| 4 | Tributylamine | 4-chlorobutanol | 80 | Thick |
| | | | | Yellow |
| | | | | Product |
| 5 | Tributylamine | 2-chloroethanol | 90 | Less Viscous |
| | | | | Liquid |
| 6 | Tripentylamine | 2-chloroethanol | 30 | |
| 7 | Tripropylamine | 5-chloropentanol | 80 | |

1D NMR Characterization:

(2-Hydroxyethyl)trimethylammonium (choline) decanoate

¹H NMR (600 MHz, DMSO) 0.80-0.87 (dt, 6H, OOCCH₂CH₂CH₂CH₂CH₂CH₂CH₂CH₂CH₃); 1.17-1.22 (m, 24H, OOCCH₂CH₂CH₂CH₂CH₂CH₂CH₂CH₂CH₃); 1.41 (h, 4H, OOCCH₂CH₂CH₂CH₂CH₂CH₂CH₂CH₂CH₃); 2.04 (q, 4H, OOCCH₂CH₂CH₂CH₂CH₂CH₂CH₂CH₂CH₃); 3.11 (s, 9H, NCH₃); 3.40 (h, 2H, NCH₂CH₂OH); 3.81 (h, 2H, NCH₂CH₂OH)

(2-Hydroxyethyl)trimethylammonium (choline) glutarate

¹H NMR (600 MHz, DMSO) 1.59-1.65 (in, 4H, OOCH₂CH₂CH₂OO); 2.07-2.20 (m, 8H, OOCH₂CH₂CH₂OO); 3.10 (s, 91-1, NCH₃); 3.39 (m, 2H, NCH₂CH₂OH); 3.82 (dtd, 2H, NCH₂CH₂OH)

(2-Hydroxyethyl)trimethylammonium (choline) glycolate

¹H NMR (600 MHz, DMSO) 3.10 (s, 9H, NCH₃); 3.39 (m, 2H, NCH₂CH₂OH); 3.66 (d, 4H, HOCH₂OO); 3.82 (m, 2H, NCH₂CH₂OH)

(2-Hydroxyethyl)trimethylammonium (choline) salicylate

¹H NMR (600 MHz, DMSO) 3.10 (s, 9H, NCH₃); 3.39 (m, 2H, NCH₂CH₂OH); 3.83 (m, 2H, NCH₂CH₂O); 6.77 (m, 4H, ArH); 7.32 (ddd, 2H, ArH); 7.74 (dd, 2H, ArH))

(2-Hydroxyethyl)trimethylammonium (choline) citronellate

¹H NMR (600 MHz, DMSO) 0.83 (m, 6H, OOCCH₂CH(CH₃)CH₂CH₂CHC(CH₃)CH₃); 1.03-1.31 (m, 4H, OOCCH₂CH(CH₃)CH₂CH₂CHC(CH₃)CH₃); 1.54 (s, 6H, OOCCH₂CH(CH₃)CH₂CH₂CHC(CH₃)CH₃); 1.62 (s, 6H, OOCCH₂CH(CH₃)CH₂CH₂CHC(CH₃)CH₃); 1.70-1.82 (m, 2H, OOCCH₂CH(CH₃)CH₂CH₂CHC(CH₃)CH₃); 1.82-1.97 (m, 4H, OOCCH2CH(CH₃)CH₂CH₂CHC(CH₃)CH₃); 1.99-2.08 (m, 4H, OOCCH₂CH(CH₃)CH₂CH₂CHC(CH₃)CH₃); 3.09 (s, 9H, NCH₃); 3.39 (m, 2H, NCH₂CH₂OH); 3.82 (m, 2H, NCH₂CH₂OH); 5.05 (tt, 2H, OOCCH₂CH(CH₃)CH₂CH₂CHC(CH₃)CH₃)

(2-Hydroxyethyl)trimethylammonium (choline) hexenoate

¹H NMR (600 MHz, DMSO)

0.83 (t, 6H, OOCCHCHCH₂CH₂CH₃); 1.36 (m, 4H, OOCCHCHCH₂CH₂CH₃); 1.99-2.08 (qd, 4H, OOCCHCHCH₂CH₂CH₃); 3.10 (s, 9H, NCH₃); 3.40 (m, 2H, NCH₂CH₂OH); 3 (m, 2H, NCH₂CH₂OH); 5.68-5.74 (dt, 2H, OOCCHCHCH₂CH₂CH₃); 6.44-6.52 (dt, 2H, OOCCHCHCH₂CH₂CH₃)

(2-Hydroxyethyl)trimethylammonium (choline) octenoate

¹H NMR (600 MHz, DMSO)

0.84 (t, 6H, OOCCHCHCH₂CH₂CH₂CH₂CH₃); 1.18-1.40 (m, 12H, OOCCHCHCH₂ CH₂CH₂CH₂CH₃); 2.01-2.10 (qd, 4H, OOCCHCHCH₂CH₂CH₂CH₂CH₃); 3.11 (s, 9H, N(H₃); 3.40 (m, 2H, NCH₂CH₂OH); 3.83 (m, 2H, NCH₂CH₂OH); 5.67-5.74 (dt, 2H, OOCCHCHCH₂CH₂CH₂CH₂CH₃); 6.46-6.55 (dt, 2H, OOCCHCCH₂ CH₂CH₂CH₂CH₃)

(2-Hydroxyethyl)trimethylammonium (choline) octanoate

¹H NMR (600 MHz, DMSO)

0.77 (t, 6H, OOCCH₂CH₂CH₂CH₂CH₂CH₂CH₃); 1.13-1.33 (m, 16H, OOCCH₂CH₂CH₂CH₂CH₂CH₂CH₃); 1.95-2.02. (q, 4H, OOCCH₂CH₂CH₂CH₂CH₂CH₂CH₃); 3.12 (s, 9H, NCH₃); 3.45 (in, 2H, NCH₂CH₂OH); 3.85 (m, 2H, NCH₂CH₂OH); 5.72 (t, 2H, OOCCH₂CH₂CH₂CH₂CH₂CH₂CH₃); 6.51 (dt, 2H, OOCCH₂CH₂CH₂CH₂CH₂CH₂CH₃);

(2-Hydroxyethyl)tributylammonium geranate

¹H NMS (600 MHz, DMSO) 0.87 (d, 9H, NCH₂CH₂CH₂CH₃); 1.23-1.33 (m, 6H, NCH₂CH₂CH₂CH₃); 1.52-1.68 (m, 18H, OOCCHC(CH₃)CH₂CH₂CHC(CH₃)CH₃; NCH₂CH₂CH₂CH₃) 2.02-2.13 (m, 14H, OOCCHC(CH₃)CH₂CH₂CHC(CH₃)CH₃); 2.89-2.97 (m, 2H, NCH₂CH₂CH₂CH₃); 3.21-3.27 (m, 4H, NCH₂CH₂CH₂CH₃); 3.31-3.36 (dd, 2H, NCH₂CH₂OH); 3.72-3.77 (t, 2H, NCH₂CH₂OH); 5.00-5.06 (dtt, 2H, OOCCHC(CH₃)CH₂CH₂CHC(CH₃)CH₃); 5.55-5.59 (m, 2H, OOCCHC(CH₃)CH₂CH₂CHC(CH₃)CH₃);

(4-Hydroxybutyl)tributylammonium geranate

¹H NMR (600 MHz, DMSO) 0.87 (d, 9H, NCH₂CH₂CH₂CH₃); 1.24-1.34 (m, 6H, NCH₂CH₂CH₂CH₃); 1.52-1.69 (m, 22H, NCH₂CH₂CH₂CH₃; NCH₂CH₂CH₂CH₂OH; OOCCHC(CH₃)CH₂CH₂CHC(CH₃)CH₃); 2.02-2.10 (m, 14H, OOCCHC(CH₃)CH₂CH₂CHC(CH₃)CH₃); 2.88-2.97 (m, 6H, NCH₂CH₂CH₂CH₃); 3.23-3.67 (m, 4H, NCH₂CH₂CH₂CH₂OH); 4.99-5.07 (m, 2H, OOCCHC(CH₃)CH₂CH₂CHC(CH₃)CH₃); 5.54-5.58 (m, 2H, OOCCHC(CH₃)CH₂CH₂CHC(CH₃)CH₃)

(2-Hydroxyethyl)triethylammonium geranate

¹H NMR (600 MHz, DMSO) 0.86 (d, 12H, OOCCHC(CH₃)CH₂CH₂CHC(CH₃)CH₃); 1.11-1.22 (m, 14H, OOCCHC(CH₃)CH₂CH₂CHC(CH₃)CH₃); 1.67 (t, 9H, NCH₂H₃); 3.03-3.10 (m, 8H, NCH₂CH₃; NCH₂CH₂OH); 3.56 (t, 2H, NCH₂CH₂OH); 4.60-4.63 (m, 2H, OOCCHC(CH₃)CH₂CH₂CHC(CH₃)CH₃); 5.22-5.25 (m, 2H, OOCCHC(CH₃)CH₂CH₂CHC(CH₃)CH₃);

(2-Hydroxyethyl)triethylammonium citronellate

¹H NMS (600 MHz, DMSO) 0.69-0.75 (d, 6H, OOCCH₂CH(CH₃)CH₂CH₂CHC(CH₃)CH₃); 0.93-1.14 (m, 8H, OOCCH₂CH(CH₃)CH₂CH₂CHC(CH₃)CH₃); 1.33-1.45 (d,12H, OOCCH₂CH(CH₃)CH₂CH₂CHC(CH₃)CH₃); 1.67-1.82 (m, 9H, NCH₂CH₃); 1.87-1.95 (m, 2H, OOCCH₂CH(CH₃)CH₂CH₂CHC(CH₃)CH₃); 2.05-2.15 (m, 4H, OOCCH₂CH(CH₃)CH₂CH₂CHC(CH₃)CH₃); 3.24-3.31 (m, 4H, NCH₂CH₃); 3.78-383 (m, 4H, NCH₂CH₃; NCH₂CH₂OH); 4.20-4.24 (m, 2H, NCH₂CH₂OH); 4.84-4.89 (m, 2H, OOCCH₂CH(CH₃)CH₂CH₂CHC(CH₃)CH₃)

(2-Hydroxyethyl)tripropylammonium geranate

¹H NMR (600 MHz, DMSO) 0.85 (t, 9H, NCH₂CH₂CH₃); 1.52-1.56 (m, 6H, NCH₂CH₂CH₃); 1.58-1.67 (m, 12H, OOCCHC(CH₃)CH₂CH₂CHC(CH₃)CH₃); 2.01-2.14 (m, 14H, OOCCHC(CH₃)CH₂CH₂CHC(CH₃)CH₃); 2.79-2.73 (m, 2H, NCH₂CH₂CH₃; NCH₂CH₂OH); 3.16-3.35 (m, 6H, NCH₂CH₂CH₃; NCH₂CH₂OH); 3.53-3.62 (m, 2H, NCH₂CH₂OH); 4.99-5.07 (m, 2H, OOCCHC(CH₃)CH₂CH₂CHC(CH₃)CH₃); 5.55-5.58 (m, 2H, OOCCHC(CH₃)CH₂CH₂CHC(CH₃)CH₃)

(5-Hydroxypentyl)tripropylammonium geranate

¹H NMR (600 MHz, DMSO) 0.83 (t, 9H, NCH₂CH₂CH₃); 1.34-1.48 (m, 12H, NCH₂CH₂CH₃; NCH₂CH₂CH₂CH₂CH₂OH); 1.53-1.64 (m, 12H, OOCCHC(CH₃)CH₂CH₂CHC(CH₃)CH₃); 2.00-2.11 (m, 14H, OOCCHC(CH₃)CH₂CH₂CHC(CH₃)CH₃); 3.33-3.63 (m, 10H, NCH₂CH₂CH₃; NCH₂CH₂CH₂CH₂CH₂OH); 5.01-5.07 (m, 2H, OOCCHC(CH₃)CH₂CH₂CHC(CH₃)CH₃); 5.54-5.58 (m, 2H, OOCCHC(CH₃)CH₂CH₂CHC(CH₃)CH₃)

(2-Hydroxyethyl)tripentylammonium geranate

¹H NMR (600 MHz, DMSO) 0.80-0.89 (m, 9H, NCH₂CH₂CH₂CH₂CH₃); 1.26-1.53 (m, 10H, NCH₂CH₂CH₂CH₂CH₃); 1.60-1.64 (m, 20H, NCH₂CH₂CH₂CH₂CH₃; OOCCHC(CH₃)CH₂CH₂CHC(CH₃)CH₃); 1.99-2.13 (m, 14H, OOCCHC(CH₃)CH₂CH₂CHC(CH₃)CH₃); 3.21-3.39 (m, 8H, NCH₂CH₂CH₂CH₂CH₃; NCH₂CH₂OH); 3.55-3.63 (t 2H, NCH₂CH₂OH); 5.01-5.07 (m, 2H, OOCCHC(CH₃)CH₂CH₂CHC(CH₃)CH₃); 5.55-5.56 (m, 2H, OOCCHC(CH₃)CH₂CH₂CHC(CH₃)CH₃);

(4-Hydroxybutyl)tripropylammonium) geranate

¹H NMR (600 MHz, DMSO) 0.85 (t, 9H, NCH₂CH₂CH₃); 1.24-1.67 (m, 22H, NCH₂CH₂CH₃; NCH₂CH₂CH₂CH₂OH; OOCCHC(CH₃)CH₂CH₂CHC(CH₃)CH₃); 2.00-2.14 (m, 14H, OOCCHC(CH₃)CH₂CH₂CHC(CH₃)CH₃); 2.71-2.92 (m, 8H, NCH₂CH₂CH₃; NCH₂CH₂CH₂CH₂OH); 3.26-3.67 (m, 2H, NCH₂CH₂CH₂CH₂OH); 5.01-5.07 (m, 2H, OOCCHC(CH₃)CH₂CH₂CHC(CH₃)CH₃); 5.54-5.63 (m, 2H, OOCCHC(CH₃)CH₂CH₂CHC(CH₃)CH₃)

### REFERENCES

[1] Prausnitz M R, Langer R. Transdermal drug delivery. Nat Biotechnol 2008; 26:1261-8. doi:10.1038/nbt.1504.
[2] Harding C R. The stratum corneum: structure and function in health and disease. Dermatol Ther 2004; 17:6-15. doi:10.12892/ejgo3153.2016.
[3] Mitragotri S, Blankschtein D, Langer R. Ultrasound-mediated transdermal protein delivery. Science (80-) 1995. doi:10.1126/science.7638603.
[4] Azagury A, Khoury L, Enden G, Kost J. Ultrasound mediated transdermal drug delivery. Adv Drug Deliv Rev 2014. doi:10.1016/j.addr.2014.01.007.
[5] Teo A L, Shearwood C, Ng K C, Lu J, Moochhala S. Transdermal microneedles for drug delivery applications. Mater Sci Eng B Solid-State Mater Adv Technol 2006. doi:10.1016/j.mseb.2006.02.008.
[6] Allen I M G, Mark R, Mcallister D V, Us N Y, Paul F, Cros M, et al. Microneedles for transdermal drug delivery. Adv Drug Deliv Rev 2004. doi:10.1016/j.addr.2003.10.023.
[7] Panchagnula R, Pillai O, Nair V B, Ramarao P. Transdermal iontophoresis revisited. Curr Opin Chem Biol 2000. doi:10.1016/S1367-5931(00)00111-3.
[8] Kanikkannan N Iontophoresis-based transdermal delivery systems. BioDrugs 2002. doi: 10.2165/00063030-200216050-00003.
[9] Schramm J, Mitragotri S. Transdermal drug delivery by jet injectors: Energetics of jet formation and penetration. Pharm Res 2002. doi:10.1023/A:1020753329492.
[10] Inoue N, Kobayashi D, Kimura M, Toyama M, Sugawara I, Itoyama S, et al. Fundamental investigation of a novel drug delivery system, a transdermal delivery system with jet injection. Int J Pharm 1996. doi:10.1016/0378-5173(96)04488-2.
[11] Finnin B C, Morgan T_{M}. Transdermal penetration enhancers: Applications, limitations, and potential. J Pharm Sci 1999. doi:10.1021/js990154g.
[12] Karande P, Jain A, Ergun K, Kispersky V, Mitragotri S. Design principles of chemical penetration enhancers for transdermal drug delivery. Proc Natl Acad Sci USA 2005; 102:4688-93. doi:10.1073/pnas.0501176102.
[13] Lane M E. Skin penetration enhancers. Int J Pharm 2013. doi:10.1016/j.ijpharm.2013.02.040.
[14] Adawiyah N, Moniruzzaman M, Hawatulaila S, Goto M. Ionic liquids as a potential tool for drug delivery systems. Medchemcomm 2016. doi:10.1039/c6md00358c.
[15] Agatemor C, Ibsen K N, Tanner E E L, Mitragotri S. Ionic liquids for addressing unmet needs in healthcare. Bioeng Transl Med 2018; 3:7-25. doi:10.1002/btm2.10083.
[16] Tanner E E L, Ibsen K N, Mitragotri S. Transdermal insulin delivery using choline-based ionic liquids (CAGE). J Control Release 2018; 286:137-44. doi:10.1016/j.jconre1.2018.07.029.
[17] Zakrewsky M, Lovejoy K S, Kern T L, Miller T E, Le V, Nagy A, et al. Ionic liquids as a class of materials for transdermal delivery and pathogen neutralization. Proc Natl Acad Sci 2014; 111:13313-8. doi:10.1073/pnas.1403995111.
[18] Zhang D, Wang H-J, Cui X-M, Wang C-X. Evaluations of imidazolium ionic liquids as novel skin permeation enhancers for drug transdermal delivery. Pharm Dev Technol 2017; 22:511-20. doi:10.3109/10837450.2015.1131718.
[19] Banerjee, A., Ibsen, K., Brown, T., Chen, R., Agatemor, C., Mitragotri S. Ionic liquids for oral insulin delivery. Proc Natl Acad Sci 2018; In Press.
[20] Banerjee A, Ibsen K, Iwao Y, Zakrewsky M, Mitragotri S. Transdermal Protein Delivery Using Choline and Geranate (CAGE) Deep Eutectic Solvent. Adv Healthc Mater 2017; 6. doi:10.1002/adhm.201601411.
[21] Terahara T, Mitragotri S, Kost J, Langer R. Dependence of low-frequency sonophoresis on ultrasound parameters; distance of the horn and intensity. Int J Pharm 2002. doi: 10.1016/S0378-5173(01)00981-4.
[22] Gill H S, Denson D D, Burris B A, Prausnitz M R. Effect of microneedle design on pain in human volunteers. Clin J Pain 2008. doi:10.1097/AJP.0b013e31816778f9.
[23] Hao J, Li S K. Mechanistic study of electroosmotic transport across hydrated nail plates: Effects of pH and ionic strength. J Pharm Sci 2008. doi:10.1002/jps.21368.
[24] Gupta, Rai, Mitragotri. Effect of Chemical Permeation Enhancers on Skin Permeability: In silico screening using Molecular Dynamics simulations. Sci Rep n.d.
[25] Notman R, Den Otter W K, Noro M G, Briels W J, Anwar J. The permeability enhancing mechanism of DMSO in ceramide bilayers simulated by molecular dynamics. Biophys J 2007. doi:10.1529/biophysj.107.104703.
[26] Notman R, Anwar J. Breaching the skin barrier—Insights from molecular simulation of model membranes. Adv Drug Deliv Rev 2013. doi:10.1016/j.addr.2012.02.011.
[27] Holman R R, Cull C A, Turner R C. A randomized double-blind trial of acarbose in type 2 diabetes shows improved glycemic control over 3 years (U.K. Prospective Diabetes Study 44). Diabetes Care 1999. doi:10.2337/diacare.22.6.960.
[28] Kelley D E, Bidot P, Freedman Z, Haag B, Podlecki D, Rendell M, et al. Efficacy and safety of acarbose in insulin-treated patients with type 2 diabetes. Diabetes Care 1998. doi:10.2337/diacare.21.12.2056.
[29] Verstovsek S, Mesa R A, Gotlib J, Levy R S, Gupta V, DiPersio J F, et al. A Double-Blind, Placebo-Controlled Trial of Ruxolitinib for Myelofibrosis. N Engl J Med 2012. doi:10.1056/NEJMoa1110557.
[30] Mesa R A, Yasothan U, Kirkpatrick P. Ruxolitinib. Nat Rev Drug Discov 2012. doi:10.1038/nrd3652.
[31] Falto-Aizpurua L, Choudhary S, Tosti A. Emerging treatments in alopecia. Expert Opin Emerg Drugs 2014. doi:10.1517/14728214.2014.974550.
[32] Craiglow B G, Tavares D, King B A. Topical ruxolitinib for the treatment of alopecia universalis. JAMA Dermatology 2016. doi:10.1001/jamadermato1.2015.4445.
[33] Bilttner H, Lau K, Spannenberg A, Werner T. Bifunctional one-component catalysts for the addition of carbon dioxide to epoxides. ChemCatChem 2015. doi:10.1002/cctc.201402816.
[34] Ibsen K N, Ma H, Banerjee A, Tanner E E L, Nangia S, Mitragotri S. Mechanism of Antibacterial Activity of Choline-Based Ionic Liquids (CAGE). ACS Biomater Sci Eng 2018. doi:10.1021/acsbiomaterials.8b00486.
[35] Karande P, Jain A, Mitragotri S. Relationships between skin's electrical impedance and permeability in the presence of chemical enhancers. J Control Release 2006; 110:307-13. doi:10.1016/jjconre1.2005.10.012.
[36] Khatun S, Castner E W. Ionic Liquid-Solute Interactions Studied by 2D NOE NMR Spectroscopy. J Phys Chem B 2015. doi:10.1021/jp509861g.
[37] Mumenthaler C, Güntert P, Braun W, Wüthrich K. Automated combined assignment of NOESY spectra and three-dimensional protein structure determination. J Biomol NMR 1997. doi: 10.1023/A: 1018383106236.
[38] Hermann T, Güntert P, Wüthrich K. Protein NMR structure determination with automated NOE-identification in the NOESY spectra using the new software ATNOS. J Biomol NMR 2002. doi:10.1023/A:1021614115432.
[39] Dratz E A, Furstenau J E, Lambert C G, Thireault D L, Rarick H, Schepers T, et al. NMR structure of a receptor-bound G-protein peptide. Nature 1993. doi:10.1038/363276a0.
[40] Blanco F J, Jimenez M A, Herranz J, Rico M, Santoro J, Nieto J L. NMR Evidence of a Short Linear Peptide That Folds into a β-Hairpin in Aqueous Solution. J Am Chem Soc 1993. doi:10.1021/ja00066a092.
[41] Giernoth R, Bröhl A, Brehm M, Lingscheid Y. Interactions in ionic liquids probed by in situ NMR spectroscopy. J Mol Liq 2014; 192:55-8. doi:10.1016/J.MOLLIQ.2013.07.010.
[42] Lingscheid Y, Arenz S, Giernoth R. Heteronuclear NOE spectroscopy of ionic liquids. ChemPhysChem 2012. doi:10.1002/cphc.201100622.
[43] Monti D, Egiziano E, Burgalassi S, Chetoni P, Chiappe C, Sanzone A, et al. Ionic liquids as potential enhancers for transdermal drug delivery. Int J Pharm 2017; 516:45-51. doi:10.10165.1JPHARM.2016.11.020.
[44] Wu X, Chen Z, Li Y, Yu Q, Lu Y, Zhu Q, et al. Improving dermal delivery of hydrophilic macromolecules by biocompatible ionic liquid based on choline and malic acid. Int J Pharm 2019. doi:10.1016/J.IJPHARM.2019.01.021.
[45] Zakrewsky M, Banerjee A, Apte S, Kern T L, Jones M R, Sesto R E D, et al. Choline and Geranate Deep Eutectic Solvent as a Broad-Spectrum Antiseptic Agent for Preventive and Therapeutic Applications. Adv Healthc Mater 2016; 5:1282-9. doi:10.1002/adhm.201600086.
[46] Valjakka-Koskela R, Hirvonen J, Mönkkönen J, Kiesvaara J, Antila S, Lehtonen L, et al. Transdermal delivery of levosimendan. Eur J Pharm Sci 2000. doi:10.1016/S0928-0987(00)00120-2.
[47] Park H J, Prausnitz M R. Lidocaine-ibuprofen ionic liquid for dermal anesthesia. AIChE J 2015; 61:2732-8. doi:10.1002/aic.14941.
[48] Berton P, Di Bona K R, Yancey D, Rizvi S A A, Gray M, Gurau G, et al. Transdermal Bioavailability in Rats of Lidocaine in the Forms of Ionic Liquids, Salts, and Deep Eutectic. ACS Med Chem Lett 2017. doi:10.1021/acsmedchemlett.6b00504.
[49] Kandimalla K, Kanikkannan N, Andega S, Singh M. Effect of fatty acids on the permeation of melatonin across rat and pig skin in-vitro and on the transepidermal water loss in rats in-vivo. JPharmPharmacol 2010; 51:783-90. doi:10.1211/0022357991773140.
[50] Kim M J, Doh H J, Choi M K, Chung S J, Shim C K, Kim D D, et al. Skin permeation enhancement of diclofenac by fatty acids. Drug Deliv 2008. doi:10.1080/10717540802006898.
[51] Kubota K, Shibata A, Yamaguchi T. The molecular assembly of the ionic liquid/aliphatic carboxylic acid/aliphatic amine as effective and safety transdermal permeation enhancers. Eur J Pharm Sci 2016. doi:10.1016/j.ejps.2016.03.002.
[52] Tanner E E L, Piston K M, Ma H, Ibsen K N, Nangia S, Mitragotri S. The Influence of Water on Choline-based Ionic Liquids. Manuscr Prep 2019.
[53] Santos de Almeida T, Julio A, Saraiva N, Fernandes A S, Araújo MEM, Baby A R, et al. Choline-versus imidazole-based ionic liquids as functional ingredients in topical delivery systems: cytotoxicity, solubility, and skin permeation studies. Drug Dev Ind Pharm 2017. doi:10.1080/03639045.2017.1349788.
[54] Kim S, Thiessen P A, Bolton E E, Chen J, Fu G, Gindulyte A, et al. PubChem substance and compound databases. Nucleic Acids Res 2016. doi:10.1093/nar/gkv951.

### Example 2

The efficacy of intestinal administration with various ionic liquid compositions was tested using insulin. Inulin was mixed with the ionic liquids indicated in FIG. 22 and administered in the intestine. Blood glucose concentrations and plasma insulin concentrations were measured. Choline-citronelic acid, choline-octanoic acid and choline-octenoic acid were particularly efficacious in intestinal delivery of insulin.

What is claimed herein is:

1. A method of administering at least one active compound, the method comprising administering the active compound in combination with at least one ionic liquid comprising:
a hydrophobic anion comprising a carboxylic acid having a pKa of at least 4.0 and a Log P of at least 1.0; and
a cation comprising a quaternary ammonium.

2. A method of reducing weight/weight gain or treating obesity, diabetes, ulcers, cancer, or fibrosis in a subject in need thereof, the method comprising administering a composition comprising at least one ionic liquid comprising:
a hydrophobic anion comprising a carboxylic acid having a pKa of at least 4.0 and a Log P of at least 1.0; and
a cation comprising a quaternary ammonium to the subject.

3. The method of claim 2, wherein the composition does not comprise a therapeutic agent other than the at least one ionic liquid.

4. The method of claim 2, wherein the composition further comprises an active compound other than the at least one ionic liquid.

5. The method of any of the preceding claims, wherein the anion has a pKa of at least 4.5.

6. The method of any of the preceding claims, wherein the anion has a pKa of at least 5.0.

7. The method of any of the preceding claims, wherein the anion has a pKa of at least 4.895.

8. The method of any of the preceding claims, wherein the anion has a pKa of 4.5-5.5.

9. The method of any of the preceding claims, wherein the anion has a pKa of 4.895-5.19.

10. The method of any of the preceding claims, wherein the anion has a Log P of at least 2.0.

11. The method of any of the preceding claims, wherein the anion has a Log P of at least 2.5.

12. The method of any of the preceding claims, wherein the anion has a Log P of at least 2.75.

13. The method of any of the preceding claims, wherein the anion has a Log P of at least 2.8.

14. The method of any of the preceding claims, wherein the anion has a Log P of 2.5-3.5.

15. The method of any of the preceding claims, wherein the anion has a Log P of 2.8-3.01.

16. The method of any of the preceding claims, wherein the anion comprises a carbon chain of at least 8 carbons.

17. The method of any of the preceding claims, wherein the anion comprises a carbon chain with an 8 carbon backbone.

18. The method of any of the preceding claims, wherein the anion is geranic acid, octenoic acid, octanoic acid, or citronellic acid.

19. The method of any of the preceding claims, wherein the anion is octenoic acid, octanoic acid, or citronellic acid.

20. The method of any of the preceding claims, wherein the anion is an alkene.

21. The method of any of the preceding claims, wherein the anion is geranic acid, octanoic acid, or citronellic acid.

22. The method of any of the preceding claims, wherein the cation has a molar mass equal to or greater than choline.

23. The method of any of the preceding claims, wherein the quarternary ammonium has the structure of NR₄⁺ and at least one R group comprises a hydroxy group.

24. The method of any of the preceding claims, wherein the quarternary ammonium has the structure of NR₄⁺ and only one R group comprises a hydroxy group.

25. The method of any of the preceding claims, wherein the cation is C1, C6, or C7.

26. The method of any of the preceding claims, wherein the cation is selected from choline, C1, C6, and C7 and the anion is citronellic acid.

27. The method of any of the preceding claims, wherein the cation is C1 and the anion is citronellic acid.

28. The method of any of the preceding claims, wherein the cation is selected from C1, C6, and C7 and the anion is geranic acid.

29. The method of any of the preceding claims, wherein the ionic liquid is choline: citronellic acid, C1: geranic acid, or C1: citronellic acid.

30. The method of any of claims 1-16, wherein the cation is selected from choline, C1, C6, and C7 and the anion is selected from citronellic acid, octanoic acid, and octenoic acid.

31. The method of any of claims 1-16, wherein the cation is choline and the anion is selected from citronellic acid, octanoic acid, and octenoic acid.

32. The method of any of claims 1-16, wherein the ionic liquid is choline: citronellic acid, choline: octanoic acid, or choline: octenoic acid.

33. The method of any of the preceding claims, wherein the ionic liquid is not CAGE.

34. The method of any of the preceding claims, wherein the ionic liquid has less than 20 cross peaks as measured by Nuclear Overhauser Effect SpectroscopY (NOESY).

35. The method of any of the preceding claims, wherein the ionic liquid has less than 10 cross peaks as measured by Nuclear Overhauser Effect SpectroscopY (NOESY).

36. The method of any of the preceding claims, wherein the ionic liquid has less than 5 cross peaks as measured by Nuclear Overhauser Effect SpectroscopY (NOESY).

37. The method of any of the preceding claims, wherein the administration is transdermal.

38. The method of any of the preceding claims, wherein the administration is transdermal, to a mucus membrane, oral, subcutaneous, intradermal, parenteral, intratumoral, or intravenous.

39. The method of claim 26, wherein the mucus membrane is nasal, oral, or vaginal.

40. The method of any of the preceding claims, wherein the administration is oral.

41. The method of any of the preceding claims, wherein the ionic liquid is at a concentration of at least 0.1% w/v.

42. The method of any of the preceding claims, wherein the ionic liquid is at a concentration of from about 10 to about 70% w/v.

43. The method of any of the preceding claims, wherein the ionic liquid is at a concentration of from about 30 to about 50% w/v.

44. The method of any of the preceding claims, wherein the ionic liquid is at a concentration of from about 30 to about 40% w/v.

45. The method of any of the preceding claims, wherein the ionic liquid comprises a ratio of cation to anion of from about 2:1 to about 1:10.

46. The method of any of the preceding claims, wherein the ionic liquid comprises a ratio of cation to anion of from about 1:1 to about 1:4.

47. The method of any of the preceding claims, wherein the ionic liquid comprises a ratio of cation to anion of about 1:2.

48. The method of any of the preceding claims, wherein the ionic liquid has a cation:anion ratio of less than 1:1.

49. The method of any of the preceding claims, wherein the active compound is hydrophobic.

50. The method of any of the preceding claims, wherein the active compound is hydrophilic.

51. The method of any of the preceding claims, wherein the active compound comprises a polypeptide.

52. The method of any of the preceding claims, wherein the active compound has a molecular weight of greater than 450.

53. The method of any of the preceding claims, wherein the active compound has a molecular weight of greater than 500.

54. The method of any of the preceding claims, wherein the active compound comprises an antibody or antibody reagent.

55. The method of any of the preceding claims, wherein the active compound comprises insulin, acarbose, ruxolitinib, or a GLP-1 polypeptide or mimetic or analog thereof.

56. The method of any of the preceding claims, wherein the combination and/or composition is administered once.

57. The method of any of the preceding claims, wherein the combination and/or composition is administered in multiple doses.

58. The method of any of the preceding claims, wherein the active compound and/or composition is provided at a dosage of 1-20 mg/kg.

59. The method of any of the preceding claims, wherein the active compound and the ionic liquid are further in combination with at least one non-ionic surfactant.

60. The method of any of the preceding claims, wherein the combination and/or composition further comprises a further pharmaceutically acceptable carrier.

61. The method of any of the preceding claims, wherein the administration is oral and the combination and/or composition is provided in a degradable capsule.

62. The method of any of the preceding claims, wherein the combination is an admixture.

63. The method of any of the preceding claims, wherein the combination and/or composition is provided in one or more nanoparticles.

64. The method of any of the preceding claims, wherein the combination is provided in the form of one or more nanoparticles comprising the active compound, the nanoparticles in solution or suspension in a composition comprising the ionic liquid.

65. A composition comprising at least one ionic liquid comprising:
a hydrophobic anion comprising a carboxylic acid having a pKa of at least 4.0 and a Log P of at least 1.0; and
a cation comprising a quaternary ammonium.

66. The composition of claim 65, wherein the anion has a pKa of at least 4.5.

67. The composition of claim 65, wherein the anion has a pKa of at least 4.895.

68. The composition of claim 65, wherein the anion has a pKa of 4.5-5.5.

69. The composition of claim 65, wherein the anion has a pKa of 4.895-5.19.

70. The composition of any of claims 65-69, wherein the anion has a pKa of at least 5.0.

71. The composition of any of claims 65-69, wherein the anion has a Log P of at least 2.0.

72. The composition of any of claims 65-69, wherein the anion has a Log P of at least 2.5.

73. The composition of any of claims 65-69, wherein the anion has a Log P of at least 2.75.

74. The composition of any of claims 65-69, wherein the anion has a Log P of at least 2.8.

75. The composition of any of claims 65-69, wherein the anion has a Log P of 2.5-3.5.

76. The composition of any of claims 65-69, wherein the anion has a Log P of 2.8-3.01.

77. The composition of any of claims 65-76, wherein the anion comprises a carbon chain of at least 8 carbons.

78. The composition of any of claims 65-76, wherein the anion comprises a carbon chain with an 8 carbon backbone.

79. The composition of any of claims 65-76, wherein the anion is geranic acid, octenoic acid, octanoic acid, or citronellic acid.

80. The composition of any of claims 65-76, wherein the anion is octenoic acid, octanoic acid, or citronellic acid.

81. The composition of any of claims 65-80, wherein the anion is an alkene.

82. The composition of any of claims 65-81, wherein the anion is geranic acid, octanoic acid, or citronellic acid.

83. The composition of any of claims 65-82, wherein the cation has a molar mass equal to or greater than choline.

84. The composition of any of claims 65-83, wherein the quarternary ammonium has the structure of NR₄⁺ and at least one R group comprises a hydroxy group.

85. The composition of any of claims 65-84, wherein the quarternary ammonium has the structure of NR₄⁺ and only one R group comprises a hydroxy group.

86. The composition of any of claims 65-85, wherein the cation is C1, C6, or C7.

87. The composition of any of claims 65-86, wherein the cation is selected from choline, C1, C6, and C7 and the anion is citronellic acid.

88. The composition of any of claims 65-87, wherein the cation is C1 and the anion is citronellic acid.

89. The composition of any of claims 65-88, wherein the cation is selected from C1, C6, and C7 and the anion is geranic acid.

90. The composition of any of claims 65-89, wherein the ionic liquid is choline: citronellic acid, C1: geranic acid, or C1: citronellic acid.

91. The composition of any of claims 65-90, wherein the cation is selected from choline, C1, C6, and C7 and the anion is selected from citronellic acid, octanoic acid, and octenoic acid.

92. The composition of any of claims 65-91, wherein the cation is choline and the anion is selected from citronellic acid, octanoic acid, and octenoic acid.

93. The composition of any of claims 65-92, wherein the ionic liquid is choline:citronellic acid, choline:octanoic acid, or choline:octenoic acid.

94. The composition of any of claims 65-93, wherein the ionic liquid is not CAGE.

95. The composition of any of claims 65-94, wherein the ionic liquid comprises a ratio of cation to anion of from about 2:1 to about 1:10.

96. The composition of any of claims 65-95, wherein the ionic liquid comprises a ratio of cation to anion of from about 1:1 to about 1:4.

97. The composition of any of claims 65-96, wherein the ionic liquid comprises a ratio of cation to anion of about 1:2.

98. The composition of any of claims 65-97, wherein the ionic liquid has a cation:anion ratio of less than 1:1.

99. The composition of any of claims 65-98, wherein the ionic liquid has a cation:anion ratio with an excess of anion.

100. The composition of any of claims 65-99, wherein the ionic liquid has less than 20 cross peaks as measured by Nuclear Overhauser Effect SpectroscopY (NOESY).

101. The composition of any of claims 65-100, wherein the ionic liquid has less than 10 cross peaks as measured by Nuclear Overhauser Effect SpectroscopY (NOESY).

102. The composition of any of claims 65-101, wherein the ionic liquid has less than 5 cross peaks as measured by Nuclear Overhauser Effect SpectroscopY (NOESY).

103. The composition of any of claims 65-102, further comprising at least one active compound in combination with the at least one ionic liquid.

104. The composition of claim 103, wherein the active compound is hydrophobic.

105. The composition of any of claims 103-104, wherein the active compound is hydrophilic.

106. The composition of any of claims 103-105, wherein the active compound comprises a polypeptide.

107. The composition of any of claims 103-106, wherein the active compound has a molecular weight of greater than 450.

108. The composition of any of claims 103-107, wherein the active compound has a molecular weight of greater than 500.

109. The composition of any of claims 103-108, wherein the active compound comprises an antibody or antibody reagent.

110. The composition of any of claims 103-109, wherein the active compound comprises insulin, acarbose, ruxolitinib, or a GLP-1 polypeptide or mimetic or analog thereof.

111. The composition of any of claims 65-110, wherein the ionic liquid is at a concentration of at least 0.1% w/v.

112. The composition of any of claims 65-111, wherein the ionic liquid is at a concentration of from about 10 to about 70% w/v.

113. The composition of any of claims 65-112, wherein the ionic liquid is at a concentration of from about 30 to about 50% w/v.

114. The composition of any of claims 65-113, wherein the ionic liquid is at a concentration of from about 30 to about 40% w/v.

115. The composition of any of claims 65-114, wherein the composition is formulated for transdermal administration.

116. The composition of any of claims 65-115, wherein the composition is formulated for administration transdermally, to a mucus membrane, orally, subcutaneously, intradermally, parenterally, intratumorally, or intravenously.

117. The composition of claim 116, wherein the mucus membrane is nasal, oral, or vaginal.

118. The composition of any of claims 65-117, wherein the composition is formulated for oral administration.

119. The composition of any of claims 103-118, wherein the active compound is provided at a dosage of 1-20 mg/kg.

120. The composition of any of claims 65-119, further comprising at least one non-ionic surfactant.

121. The composition of any of claims 65-120, further comprising a pharmaceutically acceptable carrier.

122. The composition of any of claims 65-121, wherein the composition is provided in a degradable capsule.

123. The composition of any of claims 65-122, wherein the composition is an admixture.

124. The composition of any of claims 65-123, wherein the composition is provided in one or more nanoparticles.

125. The composition of any of claims 65-124, comprising one or more nanoparticles comprising the active compound, the nanoparticles in solution or suspension in a composition comprising the ionic liquid.

126. A method of designing and/or identifying an ionic liquid comprising two ions, wherein one ion is a cation and the other ion is an anion, the method comprising:
a. selecting one of the two ions of the ionic liquid; and
b. selecting the other ion to minimize inter-ionic interactions.

127. A method of designing and/or identifying an ionic liquid comprising two ions, wherein one ion is a cation and the other ion is an anion, the method comprising:
a. selecting the cation; and
b. selecting the anion to minimize inter-ionic interactions.

128. A method of designing and/or identifying an ionic liquid comprising two ions, wherein one ion is a cation and the other ion is an anion, the method comprising:
a. selecting the anion; and
b. selecting the cation to minimize inter-ionic interactions.

129. A method of designing and/or identifying an ionic liquid comprising two ions, wherein one ion is a cation and the other ion is an anion, from a pool of candidate cations and a pool of candidate anions, the method comprising:
a. selecting one of the two ions of the ionic liquid from the pool of candidate ions; and
b. selecting from the other pool of candidate ions the ion which most minimizes inter-ionic interactions with the ion selected in step a.

130. A method of designing and/or identifying an ionic liquid comprising two ions, wherein one ion is a cation and the other ion is an anion, from a pool of candidate cations and a pool of candidate anions, the method comprising:
a. selecting the cation from the pool of candidate cations;
b. selecting from the pool of candidate anions the anion which most minimizes inter-ionic interactions with the cation selected in step a.

131. A method of designing and/or identifying an ionic liquid comprising two ions, wherein one ion is a cation and the other ion is an anion, from a pool of candidate cations and a pool of candidate anions, the method comprising:
a. selecting the cation from the pool of candidate anions;
b. selecting from the pool of candidate cations the anion which most minimizes inter-ionic interactions with the anion selected in step a.

132. The method of any of claims 126-131, wherein the ionic liquid is selected or designed for transdermal administration.

133. The method of any of claims 126-132, wherein the ionic liquid is selected or designed for administration transdermally, to a mucus membrane, orally, subcutaneously, intradermally, parenterally, intratumorally, or intravenously.

134. The method of claim 133, wherein the mucus membrane is nasal, oral, or vaginal.

135. The method of any of claims 126-134, wherein the ionic liquid is selected or designed for oral administration.

136. The method of any of claims 126-135, wherein the ionic liquid is selected or designed for delivery of an active compound.

137. The method of any of claims 126-136, wherein the cation comprises, or selecting the cation comprises selection a cation that comprises a quaternary ammonium; and the anion comprises or selecting an anion comprises selecting a hydrophobic anion comprising a carboxylic acid having a pKa of at least 4.0 and a Log P of at least 1.0.

138. The method of any of claims 126-137, wherein the anion has, or selecting an anion comprises selecting an anion that has, a pKa of at least 4.5.

139. The method of any of claims 126-138, wherein the anion has, or selecting an anion comprises selecting an anion that has, a pKa of at least 4.895.

140. The method of any of claims 126-138, wherein the anion has, or selecting an anion comprises selecting an anion that has, a pKa of 4.5-5.5.

141. The method of any of claims 126-140, wherein the anion has, or selecting an anion comprises selecting an anion that has, a pKa of 4.895-5.19.

142. The method of any of claims 126-141, wherein the anion has, or selecting an anion comprises selecting an anion that has, a pKa of at least 5.0.

143. The method of any of claims 126-142, wherein the anion has, or selecting an anion comprises selecting an anion that has, a Log P of at least 2.0.

144. The method of any of claims 126-143, wherein the anion has, or selecting an anion comprises selecting an anion that has, a Log P of at least 2.5.

145. The method of any of claims 126-144, wherein the anion has, or selecting an anion comprises selecting an anion that has, a Log P of at least 2.75.

146. The method of any of claims 126-145, wherein the anion has, or selecting an anion comprises selecting an anion that has, a Log P of at least 2.8.

147. The method of any of claims 126-146, wherein the anion has, or selecting an anion comprises selecting an anion that has, a Log P of 2.5-3.5.

148. The method of any of claims 126-147, wherein the anion has, or selecting an anion comprises selecting an anion that has, a Log P of 2.8-3.01.

149. The method of any of claims 126-148, wherein the anion comprises, or selecting an anion comprises selecting an anion that comprises, a carbon chain of at least 8 carbons.

150. The method of any of claims 126-149, wherein the anion comprises, or selecting an anion comprises selecting an anion that comprises, a carbon chain with an 8 carbon backbone.

151. The method of any of claims 126-150, wherein the anion is, or selecting an anion comprises selecting an anion that is, geranic acid, octenoic acid, octanoic acid, or citronellic acid.

152. The method of any of claims 126-151, wherein the anion is, or selecting an anion comprises selecting an anion that is, octenoic acid, octanoic acid, or citronellic acid.

153. The method of any of claims 126-152, wherein the anion is, or selecting an anion comprises selecting an anion that is, an alkene.

154. The method of any of claims 126-153, wherein the anion is, or selecting an anion comprises selecting an anion that is, geranic acid, octanoic acid, or citronellic acid.

155. The method of any of claims 126-154, wherein the cation has, or selecting a cation comprises selecting a cation that has, a molar mass equal to or greater than choline.

156. The method of any of claims 126-155, wherein the quarternary ammonium has the structure of NR₄⁺ and at least one R group comprises a hydroxy group.

157. The method of any of claims 126-156, wherein the quarternary ammonium has the structure of NR₄⁺ and only one R group comprises a hydroxy group.

158. The method of any of claims 126-157, wherein the cation is, or selecting a cation comprises selecting a cation that is, C1, C6, or C7.

159. The method of any of claims 126-158, wherein the cation is, or selecting a cation comprises selecting a cation that is, selected from choline, C1, C6, and C7 and the anion is citronellic acid.

160. The method of any of claims 126-159, wherein the cation is, or selecting a cation comprises selecting a cation that is, selected from C1, C6, and C7 and the anion is geranic acid.

161. The method of any of claims 126-160, wherein the cation is, or selecting a cation comprises selecting a cation that is, choline, C1, C6, or C7 and selecting the anion comprises selecting an anion that is citronellic acid, octanoic acid, or octenoic acid.

162. The method of any of claims 126-161, wherein the cation is choline and selecting the anion comprises selecting an anion that is citronellic acid, octanoic acid, or octenoic acid.

163. The method of any of claims 126-162, wherein the ionic liquid is not CAGE.

164. The method of any of claims 126-163, wherein the ionic liquid comprises a ratio of cation to anion of from about 2:1 to about 1:10.

165. The method of any of claims 126-164, wherein the ionic liquid comprises a ratio of cation to anion of from about 1:1 to about 1:4.

166. The method of any of claims 126-165, wherein the ionic liquid comprises a ratio of cation to anion of about 1:2.

167. The method of any of claims 126-166, wherein the ionic liquid has a cation:anion ratio of less than 1:1.

168. The method of any of claims 126-167, wherein the ionic liquid has a cation:anion ratio with an excess of anion.

169. The method of any of claims 126-168, wherein minimizing inter-ionic interaction comprises minimizing the number of cross peaks as measured by Nuclear Overhauser Effect SpectroscopY (NOESY).

170. The method of any of claims 126-169, wherein a cation and anion minimize inter-ionic interaction if they have less than 20 cross peaks as measured by Nuclear Overhauser Effect SpectroscopY (NOESY).

171. The method of any of claims 126-170, wherein a cation and anion minimize inter-ionic interaction if they have less than 10 cross peaks as measured by Nuclear Overhauser Effect SpectroscopY (NOESY).

172. The method of any of claims 126-171, wherein a cation and anion minimize inter-ionic interaction if they have less than 5 cross peaks as measured by Nuclear Overhauser Effect SpectroscopY (NOESY).

173. The method of any of claims 126-172, wherein the active compound is hydrophobic.

174. The method of any of claims 126-173, wherein the active compound is hydrophilic.

175. The method of any of claims 126-174, wherein the active compound comprises a polypeptide.

176. The method of any of claims 126-175, wherein the active compound has a molecular weight of greater than 450.

177. The method of any of claims 126-176, wherein the active compound has a molecular weight of greater than 500.

178. The method of any of claims 126-177, wherein the active compound comprises an antibody or antibody reagent.

179. The method of any of claims 126-178, wherein the active compound comprises insulin, acarbose, ruxolitinib, or a GLP-1 polypeptide or mimetic or analog thereof.

180. The method of any of claims 126-179, wherein the ionic liquid is at a concentration of at least 0.1% w/v.

181. The method of any of claims 126-180, wherein the ionic liquid is at a concentration of from about 10 to about 70% w/v.

182. The method of any of claims 126-181, wherein the ionic liquid is at a concentration of from about 30 to about 50% w/v.

183. The method of any of claims 126-182, wherein the ionic liquid is at a concentration of from about 30 to about 40% w/v.

184. The method of any of claims 126-183, wherein the active compound is provided at a dosage of 1-20 mg/kg.

185. The method of any of claims 126-184, wherein the ionic liquid is designed or selected to be provided in a degradable capsule.

186. The method of any of claims 126-185, wherein the ionic liquid and active compound are in admixture.

187. The method of any of claims 126-186, wherein the ionic liquid and optionally the active compound are selected or designed to be provided in one or more nanoparticles.

188. The method of any of claims 126-187, wherein the ionic liquid is in solution or suspension in a composition with one or more nanoparticles comprising the active compound.


SEQUENCE LISTING (4 sequences)

SEQ ID NO: 1 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 2 - protein, 37 aa, Homo sapiens
HDEFERHAEGTFTSDVSSYLEGQAAKEFIAWLVKGRG

SEQ ID NO: 3 - protein, 30 aa, Homo sapiens
HAEGTFTSDVSSYLEGQAAKEFIAWLVKGR

SEQ ID NO: 4 - protein, 31 aa, Homo sapiens
HAEGTFTSDVSSYLEGQAAKEFIAWLVKGRG